SEQUENCE LISTING (62 sequences)

SEQ ID NO: 1 - protein, 118 aa, Artificial Sequence
  VH region, 1630
EVQLLESGGGLVQPGGSLRLSCAASGFTFGYSYMSWVRQAPGKGLEWVSSIGSGSSYTYYADSVKGRFTISRDNSKNTLYLQMNSLRAEDTAVYYCARVYSSPGIDYWGQGTLVTVSS

SEQ ID NO: 2 - protein, 108 aa, Artificial Sequence
  VL region, 1630
DIQMTQSPSSLSASVGDRVTITCRASQSISSYLNWYQQKPGKAPKLLIYAASSLQSGVPSRFSGSGSGTDFTLTISSLQPEDFATYYCQQYYTWVPFTFGQGTKLEIK

SEQ ID NO: 3 - protein, 8 aa, Artificial Sequence
  CDRH1, 1630
GFTFGYSY

SEQ ID NO: 4 - protein, 8 aa, Artificial Sequence
  CDRH2, 1630
IGSGSSYT

SEQ ID NO: 5 - protein, 11 aa, Artificial Sequence
  CDRH3, 1630
ARVYSSPGIDY

SEQ ID NO: 6 - protein, 6 aa, Artificial Sequence
  CDRL1, 1631
QSISSY

SEQ ID NO: 7 - skipped

SEQ ID NO: 8 - protein, 10 aa, Artificial Sequence
  CDRL3, 1631
QQYYTWVPFT

SEQ ID NO: 9 - DNA, 354 bp, Artificial Sequence
  VH region, 1630, nucleotide
gaggtgcagctgttggagagcgggggaggcttggtacagcctggggggtccctgcgcctctcctgtgcagccagcggattcacctttggttactcttacatgtcttgggtccgccaggctccagggaaggggctggagtgggtctcatctattggttctggttcttcttacacatactatgcagactccgtgaagggccggttcaccatctcccgtgacaattccaagaacacgctgtatctgcaaatgaacagcctgcgtgccgaggacacggctgtatattattgtgcgcgcgtttactcttctccgggtattgactattggggccagggaaccctggtcaccgtctcctca

SEQ ID NO: 10 - DNA, 324 bp, Artificial Sequence
  VL region, 1630, nucleotide
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactacacttgggttccgttcacttttggccaggggaccaagctggagatcaaa

SEQ ID NO: 11 - protein, 255 aa, Homo sapiens
  human CD137
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 12 - protein, 330 aa, Homo sapiens [sequence omitted]

SEQ ID NO: 13 - protein, 327 aa, Artificial Sequence
  Modified IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 327 aa, Artificial Sequence
  Modified IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRYTQKSLSLSLGK

SEQ ID NO: 15 - protein, 327 aa, Homo sapiens
  IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 107 aa, Homo sapiens [sequence omitted]

SEQ ID NO: 17 - protein, 445 aa, Artificial Sequence
  Heavy chain, 1630
EVQLLESGGGLVQPGGSLRLSCAASGFTFGYSYMSWVRQAPGKGLEWVSSIGSGSSYTYYADSVKGRFTISRDNSKNTLYLQMNSLRAEDTAVYYCARVYSSPGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 215 aa, Artificial Sequence
  Light chain, 1631
DIQMTQSPSSLSASVGDRVTITCRASQSISSYLNWYQQKPGKAPKLLIYAASSLQSGVPSRFSGSGSGTDFTLTISSLQPEDFATYYCQQYYTWVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 118 aa, Artificial Sequence
  VH region, 2674
EVQLLESGGGLVQPGGSLRLSCAASGFNFGYSYMSWVRQAPGKGLEWVSSIGSTSSHTYYADSVKGRFTISRDNSKNTLYLQMNSLRAEDTAVYYCARVYSSPGIDYWGQGTLVTVSS

SEQ ID NO: 20 - protein, 108 aa, Artificial Sequence
  VL region, 2675
DIQMTQSPSSLSASVGDRVTITCRASQSIGSTLNWYQQKPGKAPKLLIYGASSLQSGVPSRFSGSGSGTDFTLTISSLQPEDFATYYCQQYYTWVPFTFGQGTKLEIK

SEQ ID NO: 21 - protein, 8 aa, Artificial Sequence
  CDRH1, 2674
GFNFGYSY

SEQ ID NO: 22 - protein, 8 aa, Artificial Sequence
  CDRH2, 2674
IGSTSSHT

SEQ ID NO: 23 - protein, 11 aa, Artificial Sequence
  CDRH3, 2674
ARVYSSPGIDY

SEQ ID NO: 24 - protein, 6 aa, Artificial Sequence
  CDRL1, 2675
QSIGST

SEQ ID NO: 25 - skipped

SEQ ID NO: 26 - protein, 10 aa, Artificial Sequence
  CDRL3, 2675
QQYYTWVPFT

SEQ ID NO: 27 - DNA, 1335 bp, Artificial Sequence
  VH region, 2674, nucleotide
gaggtgcagttgttggaatctggcggaggattggtgcagcctggcggatctctgagactgtcttgtgccgcctctggcttcaacttcggctactcctacatgtcctgggtccgacaggctcctggcaaaggactggaatgggtgtcctccatcggctccaccagctctcacacctactacgccgattccgtgaagggcagattcaccatcagccgggacaactccaagaacaccctgtacctgcagatgaactccctgagagccgaggacaccgccgtgtactactgtgccagagtgtactcctctcctggcatcgattattggggccagggcacactggtcaccgtgtcctctgcttctaccaagggaccctctgtgttccctctggctccttgctccagatccacctctgagtctaccgctgctctgggctgcctggtcaaggattactttcctgagcctgtgaccgtgtcttggaactccggtgctctgacatccggcgtgcacacatttccagctgtgctgcagtcctccggcctgtactctctgtcctctgtcgtgaccgtgccttctagctctctgggcaccaagacctacacctgtaacgtggaccacaagccttccaacaccaaggtggacaagcgcgtggaatctaagtacggccctccatgtccaccatgtcctgctccagaattcctcggcggaccaagcgtgttcctgtttcctccaaagcctaaggacaccctgatgatctctcggacccctgaagtgacctgcgtggtggtggatgtgtctcaagaggacccagaagtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctcgggaacctcaggtttacaccctgcctccaagccaagaggaaatgaccaagaaccaggtgtccctgacctgcctcgtgaagggattctacccttccgatatcgccgtggaatgggagtctaacggccagccagagaacaactacaagacaacccctcctgtgctggactccgacggctctttcttcctgtattctcgcctgaccgtggacaagtctcggtggcaagagggcaacgtgttctcctgctctgtgatgcacgaggccctgcacaaccactacacacagaagtccctgtctctgtccctgggcaag

SEQ ID NO: 28 - DNA, 645 bp, Artificial Sequence
  VL region, 2675, nucleotide
gacatccagatgacccagtctccatcctctctgtctgcctctgtgggcgacagagtgaccatcacctgtcgggcttctcagtccatcggcagcaccctgaactggtatcagcagaagcctggcaaggcccctaagctgctgatctatggcgctagctctctgcagtctggcgtgccctctagattttccggctctggctctggcaccgacttcaccctgacaatcagttccctgcagcctgaggacttcgccacctactactgccagcagtactacacctgggtgccctttacctttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctacagcctgtcctccacactgaccctgtctaaggccgactacgagaagcacaaggtgtacgcctgcgaagtgacccatcagggactgtctagccccgtgaccaagtccttcaacagaggcgagtgt

SEQ ID NO: 29 - protein, 445 aa, Artificial Sequence
  Heavy chain, 2674
EVQLLESGGGLVQPGGSLRLSCAASGFNFGYSYMSWVRQAPGKGLEWVSSIGSTSSHTYYADSVKGRFTISRDNSKNTLYLQMNSLRAEDTAVYYCARVYSSPGIDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 215 aa, Artificial Sequence
  Light chain, 2675
DIQMTQSPSSLSASVGDRVTITCRASQSIGSTLNWYQQKPGKAPKLLIYGASSLQSGVPSRFSGSGSGTDFTLTISSLQPEDFATYYCQQYYTWVPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 440 aa, Artificial Sequence
  Heavy chain, Nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 214 aa, Artificial Sequence
  Light chain, Nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 447 aa, Artificial Sequence
  Heavy chain, Pembrolizumab
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 218 aa, Artificial Sequence
  Light chain, Pembrolizumab
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 288 aa, Homo sapiens
  human PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 36 - protein, 290 aa, Homo sapiens
  human PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 37 - protein, 447 aa, Artificial Sequence
  Heavy chain, Pidilizumab
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 213 aa, Artificial Sequence
  Light chain, Pidilizumab
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 444 aa, Artificial Sequence
  Heavy chain, Cemiplimab
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 214 aa, Artificial Sequence
  Light chain, Cemiplimab
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 443 aa, Artificial Sequence
  Heavy chain, Spartalizumab
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 42 - protein, 220 aa, Artificial Sequence
  Light chain, Spartalizumab
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 443 aa, Artificial Sequence
  Heavy chain, Camrelizumab
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 214 aa, Artificial Sequence
  Lighty chain, Camrelizumab
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 445 aa, Artificial Sequence
  Heavy chain, Tislelizumab
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 46 - protein, 214 aa, Artificial Sequence
  Light chain, Tislelizumab
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 452 aa, Artificial Sequence
  Heavy chain, Toripalimab
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 219 aa, Artificial Sequence
  Light chain, Toripalimab
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 443 aa, Artificial Sequence
  Heavy chain, Dostarlimab
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  Light chain, Dostarlimab
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 445 aa, Artificial Sequence
  Heavy chain, INCMGA00012
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 52 - protein, 218 aa, Artificial Sequence
  Light chain, INCMGA00012
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 448 aa, Artificial Sequence
  Heavy chain, Atezolizumab
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 214 aa, Artificial Sequence
  Light chain, Atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 451 aa, Artificial Sequence
  Heavy chain, Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 215 aa, Artificial Sequence
  Light chain, Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 450 aa, Artificial Sequence
  Heavy chain, Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 216 aa, Artificial Sequence
  Light chain, Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 59 - protein, 450 aa, Artificial Sequence
  Heavy chain, CK-301 (Cosibelimab)
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 218 aa, Artificial Sequence
  Light chain, CK-301 (Cosibelimab)
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 61 - protein, 445 aa, Artificial Sequence
  Heavy chain, JTX-4014
QVQLVQSGAEVKKPGASVKVSCKASGYTFPSYYMHWVRQAPGQGLEWMGIINPEGGSTAYAQKFQGRVTMTRDTSTSTVYMELSSLRSEDTAVYYCARGGTYYDYTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 214 aa, Artificial Sequence
  Light chain, JTX-4014
DIQMTQSPSTLSASVGDRVTITCRASQSISSWLAWYQQKPGKAPKLLIYEASSLESGVPSRFSGSGSGTEFTLTISSLQPDDFATYYCQQYNSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0287199 A1
(43) Pub. Date: Aug. 29, 2024

(54) NOVEL COMBINATION THERAPIES AND USES THEREOF

(71) Applicant: ALLIGATOR BIOSCIENCE AB, Lund (SE)

(72) Inventors: Karin E. Smith, Lund (SE); Peter Ellmark, Lund (SE)

(21) Appl. No.: 18/567,827

(22) PCT Filed: Jun. 17, 2022
(86) PCT No.: PCT/EP2022/066566

§ 371 Date: Dec. 7, 2023

(30) Foreign Application Priority Data
Jun. 18, 2021 (GB) 2108712.7
Oct. 21, 2021 (GB) 2115119.6

(51) Int. Cl. C07K 16/28; A61K 39/00; A61P 35/00

(52) U.S. Cl. CPC C07K 16/2878; A61P 35/00; C07K 16/2818; A61K 2039/507

(57) ABSTRACT

The present invention relates to combination therapies for treating cancer in a subject, as well as methods for use thereof. The combination therapies comprise (a) an antibody, or antigen-binding fragment thereof, that specifically binds to CD137 and (b) a further immunotherapeutic agent, wherein the further immunotherapeutic agent is a PD-1 inhibitor. The invention also relates to pharmaceutical compositions comprising, uses of, methods of using, and kits comprising the combination therapies of the invention. The cancer may be a solid tumour.

### FIELD OF THE INVENTION

The present invention relates to combination therapies for treating cancer in a subject, as well as methods for use thereof. The combination therapies comprise (a) an antibody, or antigen-binding fragment thereof, that specifically binds to CD137 and (b) a further immunotherapeutic agent, wherein the further immunotherapeutic agent is a PD-1 inhibitor. The invention also relates to pharmaceutical compositions comprising, uses of, methods of using, and kits comprising the combination therapies of the invention. The cancer may be a solid tumour.

### BACKGROUND TO THE INVENTION

Cancer is a leading cause of premature deaths in the developed world. The aim of immunotherapy in cancer is to mount an effective immune response by the body against a tumour, particularly a solid tumour. This may be achieved by, for example, breaking tolerance against tumour antigen, augmenting anti-tumor immune responses, and stimulating local cytokine responses at the tumor site. The key effector cell of a long lasting anti-tumor immune response is the activated tumor specific effector T cell. Potent expansion of activated effector T cells can redirect the immune response towards the tumour. In this context, regulatory T cells (Treg) play a role in inhibiting the anti-tumour immunity. Depleting, inhibiting, reverting or inactivating Tregs may therefore provide anti-tumour effects and revert the immune suppression in the tumour microenvironment. Further, incomplete activation of effector T cells by, for example, dendritic cells can cause T-cell anergy, which results in an inefficient anti-tumor response, whereas adequate induction by dendritic cells can generate a potent expansion of activated effector T cells, redirecting the immune response towards the tumor. In addition, Natural killer (NK) cells play an important role in tumour immunology by attacking tumour cells with down-regulated human leukocyte antigen (HLA) expression and by inducing antibody dependent cellular cytotoxicity (ADCC). Stimulation of NK cells may thus also reduce tumour growth.

CD137 (4-1BB, TNFRSF9) is a TNF receptor (TNFR) superfamily member and is expressed on activated CD4⁺ and CD8⁺ T cells, Treg, DC, monocytes, mast cells and eosinophils. CD137 activation plays an important role in CD8⁺ T cell activation and survival (Lee et al., 2002; Pulle et al., 2006). It sustains and augments, rather than initiates, effector functions and preferentially supports Th1 cytokine production (Shuford et al., 1997). In CD4⁺ T cells, CD137 stimulation initially results in activation and later in activation-induced cell death, explaining why CD137 agonistic antibodies have shown therapeutic effect in tumour immunity as well as in autoimmunity (Zhang, J C I, 2007, Sun, Trends Mol Med, 2003). CD137 also suppresses Treg function (So, Cytokine Growth Factor Rev, 2008). Activation of CD137 is dependent on receptor oligomerization (Rabu et al., 2005; Wyzgol et al., 2009).

CD137 agonistic antibody has been shown to activate endothelial cells in the tumour environment, leading to upregulation of ICAM-1 and VCAM-1 and improved T cell recruitment (Palazon, Cancer Res, 2011).

CD137 is upregulated on NK cells activated by cytokines or CD16, in mice or humans, respectively (see Melero, CCR 19 (5)1044-53, 2013 and references cited therein). CD137 has been shown to activate NK cells in mice as well as humans, potentiating ADCC (Kohrt et al., 2014), though there are reports suggesting opposite effects on NK cells in mice and humans, leading to NK cell activation in mice and inhibition in humans (Baessler, Blood, 2010).

Several studies have demonstrated induction of tumour immunity by treatment with agonistic CD137 antibody (Dubrot et al., 2010; Gauttier et al., 2014; Kim et al., 2001; McMillin et al., 2006; Melero et al., 1997; Miller et al., 2002; Sallin et al., 2014; Taraban et al., 2002; Uno et al., 2006; Vinay and Kwon, 2012; Wilcox et al., 2002). In addition, it synergizes with several immunomodulators (Curran et al., 2011; Gray et al., 2008; Guo et al., 2013; Kwong et al., 2013; Lee et al., 2004; Morales-Kastresana et al., 2013; Pan et al., 2002; St Rose et al., 2013; Uno et al., 2006; Wei et al., 2013; Westwood et al., 2010; Westwood et al., 2014a; Westwood et al., 2014b) in pre-clinical models.

Urelumab is a strong 4-1BB agonist that has demonstrated limited clinical efficacy (Chester et al. 2017; Chin et al. 2018). Development of urelumab was however hampered by hepatotoxicity at doses ≥0.3 mg/kg (including 2 fatal events at doses ≥1 mg/kg) (Segal et al. 2017). The maximum tolerated dose was therefore set to 0.1 mg/kg (or a flat dose of 8 mg). In the subsequent studies, no clear objective responses was observed for urelumab as monotherapy (Chester et al. 2017). The mechanism behind the hepatotoxicity is not fully understood.

Utomilumab, on the other hand, is regarded as a weaker agonist than urelumab and has also shown limited clinical efficacy (Chin et al. 2018; Segal et al. 2018; Tolcher et al. 2017). Utomilumab showed a tolerable clinical safety profile up to 10 mg/kg with no dose limiting toxicity (DLT).

Utomilumab, but not urelumab, is dependent on FcγR-crosslinking to execute its agonistic effect. As FcγRs in the blood are saturated by endogenous circulating human IgG, at approximately 10 g/L, FcγR-crosslinking dependent antibodies such as utomilumab need to compete with IgG to bind to FcγRs (Jolliff 1982). Endogenous IgG of 10 g/L is more than 60-fold higher than the maximum serum concentration (Cₘₐₓ) reached with the highest clinical dose of utomilumab (155 μg/mL at 10 mg/kg) (Segal et al. 2018). The liver is a highly vascularized organ, and endogenous IgG concentrations in the liver have been shown to be similar to circulating levels (Eigenmann et al. 2017). Therefore, it can be expected that FcγR-crosslinking dependent 4-1BB activation is also reduced in the liver, due to competition with endogenous IgG. This reduced possibility for FcγR-crosslinking of utomilumab in the liver may explain the absence of liver toxicity with utomilumab that was detected with urelumab. Since 4-1BB activation with ALG.APV-527 is 5T4-crosslinking dependent and 5T4 is not expressed in liver, liver toxicity is not expected with ALG.APV-527.

Nine additional monospecific 4-1BB antibodies: ADG106, administered at doses of 0.03 to 10 mg/kg, currently in phase I/II (Liu et al. 2017), and CTX-471, AGEN2373, LVGN6051 ATOR-1017, EU101 (IND/CTA), PE0116, STA551 and HOT1030 have entered clinical development during 2018-2021 and are currently being evaluated for safety in phase I studies.

The agonistic effect of CD137 antibodies is affected by the isotype of the Fc region. The antibodies tested in the clinic are either IgG2 or IgG4. Like most TNFR family members, CD137 depends on cross linking for activation (Wilson 2011, Cancer Cell). The CD137L expressed on the membrane of an APC may induce significant multiple cross linking of the receptor. An antibody can by itself only cross link two CD137 receptors, and to induce a strong signal, further cross linking via FcγRs expressed on other cells (in trans) may be necessary for induction of a strong CD137 mediated signal. An exception to this may be IgG2 antibodies, which induce a cross linking independent signaling by an unknown mechanism (White et al, 2015 Cancer Cell). T cells do not express FcγRs, and the FcγR mediated cross linking in vivo is thought to be mediated by monocytes, macrophages, DCs and potentially B cells and other cell types.

Another factor to take into account is that engagement of FcγR receptors may also induce ADCC, antibody-dependent cellular phagocytosis (ADCP) and complement-dependent cytotoxicity (CDC) on cells coated with antibodies (for simplicity ADCC below includes ADCP and CDC). Typically, human IgG1 is a strong inducer of NK/Macrophage dependent ADCC, depending on the nature of the target, the cell type and the receptor density. IgG4 antibodies may also induce ADCC but to a lower extent than IgG1 (Wang 2015, Front Imm; Vidarson 2014 Front Imm).

The effect of a CD137 agonistic antibody with different isotypes may thus be affected by the balance between 1) inducing cross linking, which results in a stronger immune activation, and 2) inducing ADCC, which may lead to killing of both effector T cells (predominantly CD8 T cells) and Tregs. The net effect of 1) and 2) will likely depend on the distribution of CD137 expressing cells, the possibility of the target cells to engage with FcγR expressing immune cells, the receptor density and affinity and the sensitivity of Teff vs Treg to ADCC. The CD137 expression is high both on CD8 and Tregs in melanoma tumours (Quezada, presentation SITC 2015). The IgG4 format would allow for FcγRI mediated cross linking by macrophages and monocytes, yet minimizing NK mediated ADCC of effector CD8 T cells.

However, as outlined above, it is difficult to translate comparison of different human Fc in mouse models due to differences in expression and affinity between murine and human FcRs. Further, the functional consequence in vivo of antibodies blocking the binding of the CD137L to CD137 is currently debated, but it may be speculated that CD137 agonists that blocks the CD137L, and thus not allow for simultaneous activation via C137L and the CD137 agonistic antibody, have a reduced risk of inducing exaggerated activation and systemic toxicity.

Several studies have demonstrated induction of tumour immunity by treatment with agonistic CD137 mAb (Dubrot et al., 2010; Gauttier et al., 2014; Kim et al., 2001; McMillin et al., 2006; Melero et al., 1997; Miller et al., 2002; Sallin et al., 2014; Taraban et al., 2002; Uno et al., 2006; Vinay and Kwon, 2012; Wilcox et al., 2002). Two different antibodies are commonly used for in vivo studies in mice, Lob12.3 and 3H3 (Shuford 1997 J Exp Med).

The toxicity seen in mouse models has been detected following repeated dosing in a time dependent but not dose dependent manner (Ascierto 2010 Semin Onc, Dubrot 2010 Can Imm, Niu 2007 JI). The toxicity includes skin toxicity and liver toxicity: aspartate amino transferase/alanine amino transferase ratio (ASAT/ALAT) and cytokine release. This suggests that either the toxicity requires CD137 mediated pre-activation of immune cell populations (likely T cells) or it depends on secondary effects caused by antidrug-antibodies (ADA) response, potentially forming aggregations of CD137 antibodies that may lead to enhanced cross-linking. The toxicities seen in mice are reversible and seems to depend on TNFa/CD8 cell dependent manner (Ascierto 2010 Sem Onc). Toxicology studies in monkeys showed that both single and repeated dosing of up to 100 mg/kg once weekly for four weeks was tolerable with no skin or liver toxicity detected (Ascierto 2010, Semin Onc).

Prolonged and continuous activation through TNF receptor family members may lead to immune exhaustion. Therefore, it may be of advantage to administer such antibodies in a manner allowing resting periods for the cells expressing the receptors. One approach to increase the resting period in a specific dosing protocol is to reduce the half-life of an antibody by for example decreasing the binding to the neonatal Fc receptor (FcRn). This could, depending on the administration route, also reduce the toxicity associated with the treatment.

The programmed death-1 (PD-1) receptor is a negative regulator of anti-tumor T cell effector function when engaged by its ligand PD-L1, expressed on the surface of cells within a tumor (Ribas and Wolchok 2018). The PD-1 is an immune checkpoint, with its inhibitory function mediated by the tyrosine phosphatase SHP-2 that de-phosphorylates signaling molecules downstream of the T cell receptor (TCR) signaling molecules. PD-1 has two ligands, programmed death-ligand 1 (PD-L1; also known as CD274 or B7-H1), which is broadly expressed by many somatic cells mainly upon exposure to pro-inflammatory cytokines, and programmed death-ligand 2 (PD-L2, also known as CD273 or B7-DC), which has more restricted expression in antigen-presenting cells. Inflammation-induced PD-L1 expression in the tumor microenvironment results in PD-1-mediated T cell exhaustion, inhibiting the antitumor cytotoxic T cell response. PD-L1 is expressed on both tumor cells and myeloid cells. PD-1 resistance can broadly be subdivided into primary resistance or secondary (acquired) resistance. (Kluger et al. 2020). It is imperative to understand the nature of PD-1 resistance in order to select the right type of combination treatment (Yuan et al. 2021).

### SUMMARY OF THE INVENTION

Accordingly there remains a need for improved cancer therapies, in particular anti-CD137 antibodies suitable for use in treating solid tumours and combination therapies thereof.

The inventors have surprisingly found that a combination therapy comprising an anti-CD137 antibody or antigen-binding fragments thereof and a PD-1 inhibitor (such as an anti-PD-1 antibody, an anti-PD-L1 antibody or antigen binding fragments thereof) is surprisingly efficacious in the treatment of cancer.

Accordingly, a first aspect of the invention provides a combination therapy for use in the treatment or prevention of cancer in a subject comprising (a) an antibody, or antigen-binding portion thereof, that specifically binds to CD137, and (b) a further immunotherapeutic agent, wherein the further immunotherapeutic agent is a PD-1 inhibitor.

A second aspect of the invention provides an antibody, or antigen-binding portion thereof, that specifically binds to CD137 for use in a method of treating cancer, wherein the antibody or antigen-binding portion thereof that specifically binds to CD137 is for use in combination with a PD-1 inhibitor. Preferably the cancer is a solid tumour.

A related, third aspect of the invention provides the use of an antibody, or antigen-binding portion thereof, that specifically binds to CD137 in the preparation of a medicament for treating a solid tumour, wherein the antibody or antigen-binding portion thereof that specifically binds to CD137 is for use in combination with a PD-1 inhibitor.

A fourth aspect of the invention provides a pharmaceutical composition comprising (a) an antibody, or antigen-binding portion thereof, that specifically binds to CD137, and (b) a further immunotherapeutic agent, wherein the further immunotherapeutic agent is a PD-1 inhibitor.

A fifth aspect of the invention provides a kit for treating a solid tumour comprising (a) an antibody, or antigen-binding portion thereof, that specifically binds to CD137, and (b) a PD-1 inhibitor.

A sixth aspect of the invention provides a method for treating or preventing cancer, for example a solid tumour, in a subject, the method comprising administering to the subject a therapeutically effective amount of (a) administering to the subject a therapeutically effective amount of an antibody, or antigen-binding portion thereof, that specifically binds to CD137, and (b) administering to the subject a therapeutically effective amount of a PD-1 inhibitor. In some embodiments the method comprises administering the (a) an antibody, or antigen-binding portion thereof, that specifically binds to CD137, and (b) a PD-1 inhibitor simultaneously. In other embodiments the method comprises administering (a) an antibody, or antigen-binding portion thereof, that specifically binds to CD137 prior to administration of (b) a PD-1 inhibitor. In other embodiments the method comprises administering the PD-1 inhibitor prior to administration of the antibody, or antigen-binding portion thereof, that specifically binds to CD137.

In further aspects, the invention provides a PD-1 inhibitor for use in a method of treating cancer, wherein the PD-1 inhibitor is for use in combination with an antibody, or antigen-binding portion thereof that specifically binds to CD137. Preferably the cancer is a solid tumour.

A related, further aspect of the invention provides the use of a PD-1 inhibitor in the preparation of a medicament for treating a solid tumour, wherein the PD-1 inhibitor is for use in combination with an antibody, or antigen-binding portion thereof that specifically binds to CD137.

A further aspect of the invention provides a kit comprising a first isolated nucleic acid molecule encoding an antibody or antigen-binding fragment thereof that specifically binds to CD137 (or a component polypeptide chain thereof) and:
(i) a PD-1 inhibitor, and/or
(ii) a second isolated nucleic acid molecule encoding an antibody of antigen-binding fragment thereof that specifically binds to PD-1 or PD-L1 (or a component polypeptide chain thereof).

A further aspect of the invention provides a kit comprising a vector comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof.

A further aspect of the invention provides a kit comprising a host cell comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof.

A further aspect provides a kit comprising any two or more of:
a) an isolated nucleic acid molecule encoding an antibody or antigen-binding fragment thereof that specifically binds to CD137 (or a component polypeptide chain thereof);
b) a PD-1 inhibitor, and/or
c) an isolated nucleic acid molecule encoding an antibody or antigen-binding fragment thereof that specifically binds to PD-1 or PD-L1 (or a component polypeptide chain thereof);
d) an antibody, or antigen-binding portion thereof, that specifically binds to CD137;
e) a PD-1 inhibitor;
f) a vector comprising an isolated nucleic acid molecule encoding an antibody or antigen-binding fragment thereof that specifically binds to CD137 or a component polypeptide chain thereof;
g) a vector comprising an isolated nucleic acid molecule encoding an antibody or antigen-binding fragment thereof that specifically binds to PD-1 or PD-L1 or a component polypeptide chain thereof;
h) a host cell comprising an isolated nucleic acid molecule encoding an antibody or antigen-binding fragment thereof that specifically binds to CD137 or a component polypeptide chain thereof; and/or
i) a host cell comprising an isolated nucleic acid molecule encoding an antibody of antigen-binding fragment thereof that specifically binds to PD-1 or PD-L1 or a component polypeptide chain thereof.

### DETAILED DESCRIPTION OF THE INVENTION

As outlined above, a first aspect of the invention provides a combination therapy for use in the treatment or prevention of cancer in a subject comprising (a) an antibody, or antigen-binding portion thereof, that specifically binds to CD137, and (b) a further immunotherapeutic agent, wherein the further immunotherapeutic agent is a PD-1 inhibitor

In one embodiment, the cancer is a solid tumour. In an embodiment, the cancer and/or solid tumour is selected from the groups consisting of lung cancer (such as a non-small cell lung cancer (NSCLC) or a small cell lung cancer (SCLC)), a head and/or neck cancer, a gastric cancer, an oesophageal cancer, a renal cancer, a urothelial cancer, a melanoma, a breast cancer, a cervical cancer, a prostate cancer, an microsatellite instability (MSI)-high cancer, a cancer associated with DNA mismatch repair (dMMR) and/or a tumour mutational burden (TMB)-high cancer colorectal cancer; kidney cancer; pancreatic cancer; ovarian cancer; rhabdomyosarcoma; neuroblastoma; bone cancer; multiple myeloma; leukemia (such as acute lymphoblastic leukemia [ALL] and acute myeloid leukemia [AML]), skin cancer (e.g. melanoma), bladder cancer, glioblastoma, adenoma, a blastoma, a carcinoma, a desmoid tumour, a desmopolastic small round cell tumour, an endocrine tumour, a germ cell tumour, a lymphoma, a sarcoma, a Wilms tumour, a lung tumour, a colon tumour, a lymph tumour, a breast tumour and a melanoma.

In a preferred embodiment, the cancer and/or solid tumour is a lung cancer (such as a non-small cell lung cancer (NSCLC) or a small cell lung cancer (SCLC)), a head and/or neck cancer, a gastric cancer, an oesophageal cancer, a renal cancer, a urothelial cancer, a melanoma, a breast cancer, a cervical cancer, a prostate cancer, an microsatellite instability (MSI)-high cancer, a cancer associated with DNA mismatch repair (dMMR) and/or a tumour mutational burden (TMB)-high cancer, preferably wherein the cancer and/or solid tumour is metastatic.

In an embodiment, the cancer and/or solid tumour is metastatic.

### (a) Antibody Specifically Binding CD137

In an embodiment, the antibody or an antigen-binding fragment thereof (‘antibody polypeptides’) that specifically binds to CD137:
a) has binding specificity for domain 2 of human CD137;
b) is a CD137 agonist; and/or
c) is capable of inhibiting the binding of reference antibody ‘1630/1631’ to human CD137.

In some embodiments the antibody or antigen binding fragment that specifically binds to CD137 has binding specificity for domain 2 of human CD137; is a CD137 agonist; and is capable of inhibiting the binding of reference antibody ‘1630/1631’ to human CD137.

In an embodiment, the antibody or an antigen-binding fragment thereof (‘antibody polypeptides’) that specifically binds to CD137 has:
a) binding specificity for domain 2 of CD137;
b) is a CD137 agonist; and/or
c) is capable of inhibiting the binding of reference antibody ‘2674/2675’ to human CD137. In some embodiments the antibody or antigen-binding fragment thereof that specifically binds to CD137 has binding specificity for domain 2 of CD137; is a CD137 agonist; and is capable of inhibiting the binding of reference antibody ‘2674/2675’ to human CD137.

In an embodiment, the antibody or antigen binding fragment that specifically binds to CD137 is capable of inhibiting the binding of reference antibody ‘1630/1631’ and/or ‘2674/2675’ to human CD137.

In an embodiment, the antibody or antigen-binding fragment thereof that specifically binds to CD137 is capable of inhibiting the binding of one or more reference antibodies to human CD137, for example is capable of inhibiting the binding of reference antibody ‘1630/1631’ and/or ‘2674/2675’ to human CD137. Exemplary anti-CD137 antibodies are disclosed in WO 2018/091740 to Alligator Bioscience AB (the disclosures of which are incorporated herein by reference). For example, such anti-CD-137 antibodies are explicitly disclosed on pages 7-8, 11-12, 16-17 and 19 of WO 2018/091740, the disclosures of which are incorporated herein by reference.

By “CD137” we specifically include the human CD137 protein, for example as described in GenBank Accession No. AAH06196.1 (the sequence of which is set out in SEQ ID NO: 11, below). CD137 is also known in the scientific literature as 4-1BB and TNFRSF9.

Human CD137, amino acid sequence: >gi|571321|gb|AAA53133.1|4-1BB [Homo sapiens]

[SEQ ID NO: 11]
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCEL

“domain 2” as referred to above corresponds to amino acids 66 to 107 of human CD137 (see bold, underlined region in SEQ ID NO:11 above).

Thus, the combination therapy of the invention comprises an antibody or antigen-binding fragment that specifically binds to CD137 i.e. has specificity for CD137. By “specificity” we mean that the antibody polypeptide is capable of binding to CD137 in vivo, i.e. under the physiological conditions in which CD137 exists within the human body. Preferably, the antibody polypeptide does not bind to any other protein in vivo. Such binding specificity may be determined by methods well known in the art, such as ELISA, immunohistochemistry, immunoprecipitation, Western blots and flow cytometry using transfected cells expressing CD137.

The antibody or antigen-binding fragments that specifically binds to CD137 preferably binds to human CD137 with a Kd value which is less than 10×10⁻⁹M or less than 7×10⁻⁹M, more preferably less than 4, or 2×10⁻⁹M, most preferably less than 1.2×10⁻⁹M. Advantageously, the antibody polypeptide is capable of binding selectively to CD137, i.e. it bind at least 10-fold more strongly to CD137 than to any other proteins. The anti-CD137 antibody preferably specifically binds to CD137, i.e. it binds to CD137 but does not bind, or binds at a lower affinity (e.g. a 10-fold lower affinity), to other molecules (such as OX40 and/or CD40)—it therefore binds to CD137 with greater binding affinity than that at which it binds another molecule. Therefore, typically, the Kd for the antibody with respect to human CD137 will be 2-fold, preferably 5-fold, more preferably 10-fold less than Kd with respect to the other, non-target molecule, such as murine CD137, other TNFR superfamily members, or any other unrelated material or accompanying material in the environment. More preferably, the Kd will be 50-fold less, even more preferably 100-fold less, and yet more preferably 200-fold less.

Methods for measuring the overall affinity (KD) and on-rate (ka) and off-rate (kd) of an interaction (such as an interaction between an antibody and a ligand) are well known in the art. Exemplary in vitro methods are described in the accompanying Examples. It is also conceivable to use flow cytometry based methods (Sklar et al., Annu Rev Biophys Biomol Struct, (31), 97-119, 2002).

The term CD137 as used herein typically refers to human CD137. The antibody may have some binding affinity for CD137 from other mammals, such as CD137 from a non-human primate, for example Macaca fascicularis (cynomolgus monkey). The antibody preferably does not bind to murine CD137 and/or does not bind to other human TNFR superfamily members, for example human OX40 or CD40.

In an embodiment, the antibody or antigen-binding fragment thereof that specifically binds to CD137 may have affinity for CD137 in its native state, and in particular for CD137 localised on the surface of a cell.

By “localised on the surface of a cell” it is meant that CD137 is associated with the cell such that one or more region of CD137 is present on the outer face of the cell surface. For example, CD137 may be inserted into the cell plasma membrane (i.e. orientated as a transmembrane protein) with one or more regions presented on the extracellular surface. This may occur in the course of expression of CD137 by the cell. Thus, in one embodiment, “localised on the surface of a cell” may mean “expressed on the surface of a cell.” Alternatively, CD137 may be outside the cell with covalent and/or ionic interactions localising it to a specific region or regions of the cell surface.

In an embodiment, the antibodies and antigen-binding fragments thereof that specifically bind to CD137 as defined herein are CD137 agonists. For example, they may be capable of inducing the release of interferon-gamma from CD8+ T cells. Agonistic activity of anti-CD137 antibodies may be evaluated in a T cell assay based on primary CD8+ T cells (see Examples).

Thus, the antibody or antigen-binding fragment that specifically binds to CD137 may modulate the activity of a cell expressing CD137, wherein said modulation is an increase or decrease in the activity of said cell. The cell is typically a T cell. The antibody may increase the activity of a CD4+ or CD8+ effector cell, or may decrease the activity of, or deplete, a regulatory T cell (T reg). In either case, the net effect of the antibody will be an increase in the activity of effector T cells, particularly CD4+, CD8+ or NK effector T cells. Methods for determining a change in the activity of effector T cells are well known and are as described earlier.

The antibody or antigen-binding fragment that specifically binds to CD137 preferably causes an increase in activity in a CD8+ T cell in vitro, optionally wherein said increase in activity is an increase in proliferation, IFN-γ production and/or IL-2 production by the T cell. The increase is preferably at least 2-fold, more preferably at least 10-fold and even more preferably at least 25-fold higher than the change in activity caused by an isotype control antibody measured in the same assay.

As outlined above, antibodies or antigen-binding fragments thereof which are capable of inhibiting the binding of one or more reference antibodies to human CD137 are provided. The reference antibodies described herein are reference antibody 1630/1631 and reference antibody 2674/2675.

Exemplary anti-CD137 antibodies are disclosed in WO 2018/091740 to Alligator Bioscience AB (the disclosures of which are incorporated herein by reference).

By reference antibody “1630/1631” we mean an intact IgG antibody comprising heavy and light chains having the amino acid sequences of SEQ ID NOS: 17 and 18, respectively.

1630/1631-Full sequence Heavy chain
[SEQ ID NO: 17]
EVQLLESGGGLVQPGGSLRLSCAASGFTFGYSYMSWVRQAPGKGLEWVS
SIGSGSSYTYYADSVKGRFTISRDNSKNTLYLQMNSLRAEDTAVYYCAR
VYSSPGIDYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK
1630/1631-Full sequence Light chain
[SEQ ID NO: 18]
DIQMTQSPSSLSASVGDRVTITCRASQSISSYLNWYQQKPGKAPKLLIY
AASSLQSGVPSRFSGSGSGTDFTLTISSLQPEDFATYYCQQYYTWVPFT
FGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

By reference antibody “2674/2675” we mean an intact IgG antibody comprising heavy and light chains having the amino acid sequences of SEQ ID NOS: 29 and 30, respectively. Antibody 2674/2675 is also known as ATOR-1017 and these terms are fully interchangeable.

2674/2675-Full sequence heavy chain
[SEQ ID NO: 29]
EVQLLESGGGLVQPGGSLRLSCAASGFNFGYSYMSWVRQAPGKGLEWVS
SIGSTSSHTYYADSVKGRFTISRDNSKNTLYLQMNSLRAEDTAVYYCAR
VYSSPGIDYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK
2674/2675-Full sequence light chain
[SEQ ID NO: 30]
DIQMTQSPSSLSASVGDRVTITCRASQSIGSTLNWYQQKPGKAPKLLIY
GASSLQSGVPSRFSGSGSGTDFTLTISSLQPEDFATYYCQQYYTWVPFT
FGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

As discussed below, the reference antibody ‘1630/1631’ binds to domain 2 of CD137. Reference antibody 2674/2675 also binds to domain 2 of CD137. Thus, it will be appreciated that the antibody or an antigen-binding fragment thereof that specifically binds CD137 in the combination therapy of the invention also binds to domain 2 of CD137. Accordingly in some embodiments the antibody or antigen-binding fragment thereof that specifically binds to CD137 binds to domain 2 of CD137.

By “capable of inhibiting the binding of reference antibody ‘1630/1631’ to human CD137” we mean that the presence of the antibody polypeptides of the combination therapy of the invention inhibits, in whole or in part, the binding of ‘1630/1631’ to human CD137. Similarly, by “capable of inhibiting the binding of reference antibody ‘2674/2675’ to human CD137” we mean that the presence of the antibody polypeptides of the combination therapy of invention inhibits, in whole or in part, the binding of ‘2674/2675” to human CD137. The anti-CD137 antibodies or fragments thereof used in the combination therapies of the invention may therefore compete for binding to human CD137 with ‘reference antibody’ 1630/1631 and/or with ‘reference antibody’ 2674/2675. Such competitive binding inhibition can be determined using assays and methods well known in the art, for example using BIAcore chips with immobilised CD137 and incubating in the presence the reference antibody ‘1630/1631’ or ‘2674/2675’ with and without an antibody polypeptide to be tested. Alternatively, a pair-wise mapping approach can be used, in which the reference antibody ‘1630/1631’ or ‘2674/2675’ is immobilised to the surface of the BIAcore chip, CD137 antigen is bound to the immobilised antibody, and then a second antibody is tested for simultaneous CD137-binding ability (see ‘BIAcore Assay Handbook’, GE Healthcare Life Sciences, 29-0194-00 AA 05/2012; the disclosures of which are incorporated herein by reference).

In a further alternative, competitive binding inhibition can be determined using flow cytometry. For example, to test whether a test antibody is able to inhibit the binding of the 1630/1631 or 2674/2675 reference antibody to a cell surface antigen, cells expressing the antigen can be pre-incubated with the test antibody for 20 min before cells are washed and incubated with the reference 1630/1631 or 2674/2675 antibody conjugated to a fluorophore, which can be detected by flow cytometry. If the pre-incubation with the test antibody reduces the detection of the reference 1630/1631 or 2674/2675 antibody in flow cytometry, the test antibody inhibits the binding of the reference antibody to the cell surface antigen. If the antibody to be tested exhibits high affinity for CD137, then a reduced pre-incubation period may be used (or even no pre-incubation at all).

In a further alternative, competitive binding inhibition can be determined using an ELISA.

In some embodiments, the antibodies and antigen binding fragments that specifically bind CD137 of the combination therapy of the invention are defined by reference to the variable regions of reference antibodies 1630/1631 and 2674/2675.

The reference antibody designated ‘1630/1631’ comprises:
(a) a heavy chain variable region having the amino acid sequence of SEQ ID NO: 1:

[SEQ ID NO: 1]
EVQLLESGGGLVQPGGSLRLSCAASGFTFGYSYMSWVRQAPGKGLEWVS
SIGSGSSYTYYADSVKGRFTISRDNSKNTLYLQMNSLRAEDTAVYYCAR
VYSSPGIDYWGQGTLVTVSS

and
(b) a light chain variable region having the amino acid sequence of SEQ ID NO: 2:

[SEQ ID NO: 2]
DIQMTQSPSSLSASVGDRVTITCRASQSISSYLNWYQQKPGKAPKLLIY
AASSLQSGVPSRFSGSGSGTDFTLTISSLQPEDFATYYCQQYYTWVPFT
FGQGTKLEIK

The reference antibody designated ‘2674/2675’ comprises:
(a) a heavy chain variable region having the amino acid sequence of SEQ ID NO: 19:

[SEQ ID NO: 19]
EVQLLESGGGLVQPGGSLRLSCAASGFNFGYSYMSWVRQAPGKGLEWVS
SIGSTSSHTYYADSVKGRFTISRDNSKNTLYLQMNSLRAEDTAVYYCAR
VYSSPGIDYWGQGTLVTVSS

and
(b) a light chain variable region having the amino acid sequence of SEQ ID NO: 20:

[SEQ ID NO: 20]
DIQMTQSPSSLSASVGDRVTITCRASQSIGSTLNWYQQKPGKAPKLLIY
GASSLQSGVPSRFSGSGSGTDFTLTISSLQPEDFATYYCQQYYTWVPFT
FGQGTKLEIK

The term “amino acid” as used herein includes the standard twenty genetically-encoded amino acids and their corresponding stereoisomers in the ‘D’ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the antibody polypeptides as defined herein comprise or consist of L-amino acids.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including glycine and both the D or L optical isomers, and amino acid analogs and peptidomimetics.

It will be appreciated by persons skilled in the art that the binding specificity of an antibody or antigen-binding fragment thereof is conferred by the presence of complementarity determining regions (CDRs) within the variable regions of the constituent heavy and light chains, such as those CDRs described herein.

It will be further appreciated by persons skilled in the art that any intact IgG antibody comprising the above variable regions may be used as the reference antibody to identify antibody polypeptides of the combination therapy of the invention that competitively inhibit 1630/1631 or 2674/2675 binding to CD137. Preferably however, reference antibody 1630/1631 consists of heavy and light chains as defined in SEQ ID NOs:17 and 18, respectively, and reference antibody 2674/2675 consists of heavy and light chains as defined in SEQ ID NOs:29 and 30, respectively

Competitive binding typically arises because the test antibody binds at, or at least very close to, the epitope on the antigen to which binds the reference antibody (in this case, 1630/1631 or 2674/2675). However, it will be appreciated by persons skilled in the art that competitive binding may also arise by virtue of steric interference; thus, the test antibody may bind at an epitope different from that to which the reference antibody binds but may still be of sufficient size or configuration to hinder the binding of the reference antibody to the antigen.

The antibodies and antigen-binding fragments that bind to CD137 and are part of the combination therapy of the present invention were identified after screening of anti-CD137 antibodies, on the basis of exhibiting properties that make them particularly suitable as diagnostic and therapeutic agents for cancer.

Thus, in one embodiment, the antibody or antigen-binding fragment that specifically binds to CD137 exhibits one or more of the following properties:
a) the ability to stimulate CD137 and activate T cells and other immune cells via a cross-linking dependent mechanism (e.g. to induce release of interferon-gamma from CD8+ T cells; see Examples); and/or
b) cross-reactivity with cynomolgus CD137 (see Examples).

For example, the antibody or antigen-binding fragment that specifically binds to CD137 may exhibit both of the above properties. The antibody may be or may comprise a variant or a fragment of one of the specific anti-CD137 antibodies disclosed herein, provided that said variant or fragment retains specificity for CD137, and in some embodiments retains at least one of functional characteristics (a) to (b) above.

As described above, the antibodies that specifically bind to CD137 and comprise part of the combination therapy of the invention may have a cross linking dependent mechanism. By “cross linking dependent mechanism”, we include an Fc cross linking dependent mechanism wherein the antibody has to bind both CD137 and an Fc receptor in order to stimulate CD137. As such, in some embodiments the antibody has to be capable of binding both CD137 and an Fc receptor.

In an embodiment, the antibody or antigen binding domain that specifically binds to CD137 is capable of binding an Fc receptor. In one embodiment, the antibody or antigen binding domain is capable of simultaneous binding to CD137 and a Fc receptor. In a preferred embodiment, the ability of the antibody and/or antigen binding domain thereof that specifically binds to CD137 to activate T cells is dependent upon binding to both CD137 and Fc receptors.

In a preferred embodiment, the Fc receptor that is targeted is an FcγR. Examples of FcγRs include, FcγRI, FcγRIIA and FcγRIIB Thus, in one embodiment, the FcγR may be FcγRIIA. By FcγRIIA, we include both the R131 and H131 allotypes of FcγRIIA. Thus, in one embodiment, the FcγR to be targeted is the R131 allotype of FcγRIIA.

In an alternative embodiment, the antibody that specifically binds to CD137 could be Fc crosslinking independent, such that it can stimulate CD137 in the absence of binding to an Fc receptor.

Thus, exemplary antibodies 2674/2675 and 1630/1631 are FcγR-crosslinking dependent agonistic antibodies targeting the co-stimulatory CD137 receptor. They are therefore only active in tissues or tumours containing cells expressing CD137 and FcγR. By “tumours containing cells expressing CD137 and FcγR” we include tumours or tumour draining lymph nodes comprising tumour cells and/or tumour infiltrating immune cells (such as monocytes, macrophages, dendritic cells, NK cells, T cells, B cells and granulocytes) expressing CD137 and FcγR. It will be appreciated that CD137 and FcγR may be expressed on separate cells within the tumour and/or co-expressed in the same cells. Reference antibodies 2674/2675 and 1630/1631 will thus provide a tumour directed immune activation in indications associated with cells that express both CD137 and FcγR in the tumour microenvironment; this contrasts with FcγR independent CD137 agonists (e.g. Urelumab), which capable of inducing systemic immune activation. The tumour localizing effect of antibodies 2674/2675 and 1630/1631 will primarily depend on the number of tumour infiltrating macrophages/myeloid cells expressing different FcγRs.

It is known that IgG4 binds with high affinity to FcγRI and with moderate/low affinity to FcγRIIa and FcγRIIb. FcγRI and FcγRIIa are expressed on monocytes and FcγRIIb is expressed with a high density on B cells. Crosslinking of antibodies 2674/2675 and 1630/1631 will preferentially occur intratumorally as well as in adjacent draining lymph nodes. Systemically in the blood, where serum IgG levels are high, the availability of free non-blocked FcγRs are believed to be too low for an effective crosslinking to occur. Therefore, the risk for a systemic immune activation of is believed to be low which improves the risk-benefit profile compared to other CD137 mAbs.

Patient selection and a biomarker rationale for treatment with antibodies that specifically bind CD137 and form part of the combination therapy of the invention, such as 2674/2675 and 1630/1631, may be guided by tumour types that have infiltrating cells expressing CD137 and FcγRs. Thus, the combination therapy of the invention may be for use in patients selected on the basis of having a tumour containing cells expressing CD137 and FcγRs (i.e. a as companion diagnostic test).

By “infiltrating cells” we include tumour infiltrating immune cells such as monocytes, macrophages, dendritic cells, NK cells, T cells, B cells and granulocytes

Advantageously, the antibody or antigen-binding fragment thereof that specifically binds to CD137 is capable of inducing tumour immunity. Tumour immunity can be demonstrated using methods well known in the art, for example by re-challenging mice that have been cured from a given tumour by CD317 antibody treatment with the same tumour and/or by re-challenging mice that have been cured from a given tumour by the combination therapy of the present invention with the same tumour. If tumour immunity has been induced by the antibody therapy and/or combination therapy, then the tumour is rejected upon re-challenge.

In one embodiment, the antibody or antigen binding fragment thereof that specifically binds to CD137 is substantially incapable of inducing the following upon binding to cells expressing CD137:
a) antibody-dependent cellular cytotoxicity (ADCC);
b) antibody-dependent cellular phagocytosis (ADCP); and/or
c) complement-dependent cytotoxicity (CDC).

The antibody may be or may comprise a variant or a fragment of one of the specific anti-CD137 antibodies disclosed herein, provided that said variant or fragment retains specificity for CD137 and is incapable of inducing one or more of (a) to (c) upon binding to cells expressing CD137.

Methods for determining the level of ADCC-mediated lysis or apoptosis in a sample of cells are well known in the art. For example, a chromium-51 release assay, europium release assay or sulphur-35 release assay may be used. In such assays, a previously labelled target cell line expressing the antigen is incubated with an antibody to be tested. After washing, effector cells (typically expressing Fc receptor CD16) are co-incubated with the antibody-labelled target cells. Target cell lysis is subsequently measured by release of intracellular label by a scintillation counter or spectrophotometry. As an alternative to the labelling with radioisotopes required in such assays, methods may be used in which lysis is detected by measuring the release of enzymes naturally present in the target cells. This may be achieved by detection (for example bioluminescent detection) of the products of an enzyme-catalysed reaction. No previous labelling of the cells is required in such an assay. A typical cellular enzyme detected with such an assay is GAPDH.

Methods for determining the level of ADCP in a sample of cells are well known in the art. For example, the tumor antigen-expressing cancer cells may be incubated in the presence of a titration of mAb and the human leukemia monocytic cell line THP-1. Both effector and target cells may be fluorescently labelled and cell engulfment may be measured by flow cytometry. Phagocytosis may also be confirmed using microscopy or imaging cytometry.

Methods for determining the level of CDC in a sample of cells are well known in the art. For example, serum comprising the components of the complement system (typically human serum) may be mixed with target cells bound by the antibody being detected, and then cell death may be determined by a suitable method. Cell death may be determined via pre-loading the target cells with a radioactive compound. As cells die, the radioactive compound is released from them. Hence, the efficacy of the antibody to mediate cell death is may be determined by the radioactivity level. Non-radioactive CDC assays may also be used, which may determine the release of abundant cell components, such as GAPDH, with fluorescent or luminescent determination.

In one embodiment, the antibody or antigen-binding fragment thereof that specifically binds to CD137 is capable of binding to an epitope on the extracellular domain of CD137 which overlaps, at least in part, with the epitope on CD137 to which reference antibody 1630/1631 and/or 2674/2675 is capable of binding. Thus, the antibody or antigen-binding fragment may be capable of binding to an epitope located at/within domain 2 of CD137 (i.e. amino acids 66 to 107 of human CD137).

In one embodiment, the antibody or antigen-binding fragment thereof that specifically binds to CD137 comprises or consists of an intact antibody ((for example an IgG1, IgG2, IgG3 or IgG4 antibody). In a preferred embodiment, the antibody is an IgG4 antibody.

In an alternative embodiment, the antibody or antigen-binding fragment thereof that specifically binds to CD137 comprises or consists of an antigen-binding fragment selected from the group consisting of Fv fragments (e.g. single chain Fv and disulphide-bonded Fv), Fab-like fragments (e.g. Fab fragments, Fab′ fragments and F(ab)2 fragments) and domain antibodies (e.g. single V_{H} variable domains or V_{L} variable domains). In particular, the antibody or antigen-binding fragment thereof that specifically binds to CD137 may be a scFv.

In a further embodiment, as discussed above, the antibody or antigen-binding fragment thereof that specifically binds to CD137 comprises or consists of an antibody mimic selected from the group comprising or consisting of affibodies, tetranectins (CTLDs), adnectins (monobodies), anticalins, DARPins (ankyrins), avimers, iMabs, microbodies, peptide aptamers, Kunitz domains and affilins.

In one embodiment, the antibody or antigen binding fragment thereof that specifically binds to CD137 comprises:
a) a heavy chain CDR1 sequence with the consensus sequence G, F, T/N, F, G, Y, S, Y;
b) a heavy chain CDR2 sequence with the consensus sequence I, G, S, G/T, S, S, Y/H, T; and
c) a heavy chain CDR3 sequence with the sequence ARVYSSPGIDY.

In one embodiment, the antibody or antigen binding fragment thereof that specifically binds to CD137 comprises:
a) a light chain CDR1 sequence with the consensus sequence Q, S, I, S/G, S, Y/T;
b) a light chain CDR2 sequence with the consensus sequence A/G, A, S; and
c) a light chain CDR3 sequence with the sequence QQYYTWVPFT.

In a preferred embodiment, the antibody or antigen-binding fragment thereof that specifically binds to CD137 comprises a heavy chain variable region comprising the following CDRs:
a) GFTFGYSY [SEQ ID NO: 3] or an amino acid sequence containing up to 3 amino acid mutations compared to SEQ ID NO: 3, for example 1, 2 or 3 mutations;
b) IGSGSSYT [SEQ ID NO: 4] or an amino acid sequence containing up to 3 amino acid mutations compared to SEQ ID NO: 4, for example 1, 2 or 3 mutations; and
c) ARVYSSPGIDY [SEQ ID NO: 5] or an amino acid sequence containing up to 3 amino acid mutations compared to SEQ ID NO: 5, for example 1, 2 or 3 mutations.

Thus, the antibody or antigen-binding fragment thereof that specifically binds to CD137 may comprise a heavy chain variable region comprising one, two or all three of the CDRs of SEQ ID NOs 3, 4 and 5.

For example, the antibody or antigen-binding fragment thereof may comprise a heavy chain variable region having the amino acid sequence of the corresponding region of the 1630/1631 reference antibody, i.e. SEQ ID NO:1.

In an alternative preferred embodiment, the antibody or antigen-binding fragment thereof according to the first or second aspect of the invention comprises a heavy chain variable region comprising the following CDRs:
a) GFNFGYSY [SEQ ID NO: 21] or an amino acid sequence containing up to 3 amino acid mutations compared to SEQ ID NO: 21, for example 1, 2 or 3 mutations;
b) IGSTSSHT [SEQ ID NO: 22] or an amino acid sequence containing up to 3 amino acid mutations compared to SEQ ID NO: 22, for example 1, 2 or 3 mutations; and
c) ARVYSSPGIDY [SEQ ID NO: 23] or an amino acid sequence containing up to 3 amino acid mutations compared to SEQ ID NO: 23, for example 1, 2 or 3 mutations.

Thus, the antibody or antigen-binding fragment thereof that specifically binds CD137 may comprise a heavy chain variable region comprising one, two or all three of the CDRs of SEQ ID NOs 21, 22 and 23.

For example, the antibody or antigen-binding fragment thereof that specifically binds to CD137 may comprise a heavy chain variable region having the amino acid sequence of the corresponding region of the 2674/2675 reference antibody, i.e. SEQ ID NO: 19.

However, it will be appreciated (in relation to either embodiment, 1630/1631 or 2674/2675) that a low level of mutation (typically, just one, two or three amino acids) within a CDR sequence may be tolerated without loss of the specificity of the antibody or antigen-binding fragment for CD137.

For example, in an alternative embodiment, the antibody or antigen-binding fragment thereof that specifically binds to CD137 may comprise a heavy chain variable region comprising the CDRs as defined above, wherein the H1 and H2 CDRs are mutated versions of SEQ ID NO: 3 and 4, respectively, and wherein the H3 CDR is SEQ ID NO: 5.

In a further alternative embodiment, the antibody or antigen-binding fragment thereof that specifically binds to CD137 may comprise a heavy chain variable region comprising the CDRs as defined above, wherein the H1 and H2 CDRs are mutated versions of SEQ ID NO: 21 and 22, respectively, and wherein the H3 CDR is SEQ ID NO: 23.

In some embodiments the antibody or antigen-binding fragment thereof that specifically binds to CD137 comprises a heavy chain variable region having the amino acid sequence of SEQ ID NO 1: or an amino acid sequence having at least 60% sequence identity therewith, for example at least 70%, 80%, 85%, 90%, 95%, 96%, 97%, 98% or 99% sequence identity.

Percent sequence identity can be determined by, for example, the LALIGN program (Huang and Miller, Adv. Appl. Math. (1991) 12:337-357, the disclosures of which are incorporated herein by reference) at the Expasy facility site (http://www.ch.embnet.org/software/LALIGN_form.html) using as parameters the global alignment option, scoring matrix BLOSUM62, opening gap penalty −14, extending gap penalty −4. Alternatively, the percent sequence identity between two polypeptides may be determined using suitable computer programs, for example the GAP program of the University of Wisconsin Genetic Computing Group and it will be appreciated that percent sequence identity is calculated in relation to polypeptides whose sequence has been aligned optimally.

The alignment may alternatively be carried out using the Clustal W program (as described in Thompson et al., 1994, Nucl. Acid Res. 22:4673-4680, which is incorporated herein by reference). The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

In a further preferred embodiment, the antibody or antigen-binding fragment thereof that specifically binds to CD137 according to the first aspect of the invention comprises a light chain variable region comprising the following CDRs:
a) QSISSY [SEQ ID NO: 6] or an amino acid sequence containing up to 3 amino acid mutations compared to SEQ ID NO: 6, for example 1, 2 or 3 mutations;
b) AAS [SEQ ID NO: 7] or an amino acid sequence containing up to 2 amino acid mutations compared to SEQ ID NO: 7; for example 1 or 2 mutations and
c) QQYYTWVPFT [SEQ ID NO: 8] or an amino acid sequence containing up to 3 amino acid mutations compared to SEQ ID NO: 8, for example 1, 2 or 3 mutations.

Thus, the antibody or antigen-binding fragment thereof that specifically binds to CD137 may comprise a light chain variable region comprising the CDRs of SEQ ID NOs 6, 7 and 8.

For example, the antibody or antigen-binding fragment thereof that specifically binds to CD137 may comprise a light chain variable region having the amino acid sequence of the corresponding region of the 1630/1631 reference antibody, i.e. SEQ ID NO: 2 or an amino acid sequence having at least 60% sequence identity therewith, for example at least 70%, 80%, 85%, 90%, 95%, 96%, 97%, 98% or 99% sequence identity.

In an alternative embodiment, the antibody or antigen-binding fragment thereof that specifically binds to CD137 may comprise a light chain variable region comprising the CDRs as defined above, wherein the L1 and L2 CDRs are mutated versions of SEQ ID NO: 6 and 7, respectively, and wherein the L3 CDR is SEQ ID NO:8.

In a further preferred embodiment, the antibody or antigen-binding fragment thereof that specifically binds to CD137 and may comprise part of the combination therapy of the first aspect of the invention comprises a light chain variable region comprising the following CDRs:
a) QSIGST [SEQ ID NO: 24] or an amino acid sequence containing up to 3 amino acid mutations compared to SEQ ID NO: 24, for example 1, 2 or 3 mutations;
b) GAS [SEQ ID NO: 25] or an amino acid sequence containing up to 2 amino acid mutations compared to SEQ ID NO: 25; for example 1 or 2 mutations and
c) QQYYTWVPFT [SEQ ID NO: 26] or an amino acid sequence containing up to 3 amino acid mutations compared to SEQ ID NO: 26, for example 1, 2 or 3 mutations.

Thus, the antibody or antigen-binding fragment thereof that specifically binds to CD137 may comprise a light chain variable region comprising the CDRs of SEQ ID NOs 24, 25 and 26.

For example, the antibody or antigen-binding fragment thereof that specifically binds to CD137 may comprise a light chain variable region having the amino acid sequence of the corresponding region of the 2674/2675 reference antibody, i.e. SEQ ID NO: 20.

In an alternative embodiment, the antibody or antigen-binding fragment thereof that specifically binds to CD137 may comprise a light chain variable region comprising the CDRs as defined above, wherein the L1 and L2 CDRs are mutated versions of SEQ ID NO: 24 and 25, respectively, and wherein the L3 CDR is SEQ ID NO: 26.

An anti-CD137 antibody used in the combination therapies and methods of the invention may be an antibody comprising one, two or all three of the CDR sequences of SEQ ID NOs: 3 to 5 and/or one, two, or all three of the CDR sequences of SEQ ID NOs: 6 to 8. The antibody may comprise all six CDR sequences of SEQ ID NOs: 3 to 8.

The antibody may comprise or consist of the light chain variable region sequence of SEQ ID NO: 2 and/or the heavy chain variable region sequence of SEQ ID NO: 1, or an amino acid sequence having at least 60% sequence identity therewith, for example at least 70%, 80%, 85%, 90%, 95%, 96%, 97%, 98% or 99% sequence identity to SEQ ID NO: 1 and/or SEQ ID NO:2.

The antibody may be, or may bind to the same epitope as, an antibody comprising the light chain variable region sequence of SEQ ID NO: 2 and the heavy chain variable region sequence of SEQ ID NO: 1. In addition, the antibody may comprise the light chain constant region sequence of SEQ ID NO: 16 and/or the heavy chain constant region sequence of SEQ ID NO: 13, or an amino acid sequence having at least 60% sequence identity therewith, for example at least 70%, 80%, 85%, 90%, 95%, 96%, 97%, 98% or 99% sequence identity to SEQ ID NO: 16 and/or SEQ ID NO: 13.

An anti-CD137 antibody used in the combination therapies and methods of the invention may be an antibody comprising one, two or all three of the CDR sequences of SEQ ID NOs: 21 to 23 and/or one, two, or all three of the CDR sequences of SEQ ID NOs: 24 to 26. The antibody may comprise all six CDR sequences of SEQ ID NOs: 21 to 26.

The antibody may comprise the light chain variable region sequence of SEQ ID NO: 20 and/or the heavy chain variable region sequence of SEQ ID NO: 19 or an amino acid sequence having at least 60% sequence identity therewith, for example at least 70%, 80%, 85%, 90%, 95%, 96%, 97%, 98% or 99% sequence identity to SEQ ID NO: 20 and/or 19.

The antibody may be, or may bind to the same epitope as, an antibody comprising the light chain variable region sequence of SEQ ID NO: 20 and the heavy chain variable region sequence of SEQ ID NO: 19. In addition, the antibody may comprise the light chain constant region sequence of SEQ ID NO: 16 and/or the heavy chain constant region sequence of SEQ ID NO: 13 or an amino acid sequence having at least 60% sequence identity therewith, for example at least 70%, 80%, 85%, 90%, 95%, 96%, 97%, 98% or 99% sequence identity to SEQ ID NO: 16 and/or 13.

It will be appreciated by persons skilled in the art that for human therapy, human or humanised antibodies are preferably used. Humanised forms of non-human (e.g. murine) antibodies are genetically engineered chimaeric antibodies or antibody fragments having preferably minimal-portions derived from non-human antibodies.

Humanised antibodies include antibodies in which complementary determining regions of a human antibody (recipient antibody) are replaced by residues from a complementary determining region of a non-human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596, the disclosures of which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567, the disclosures of which are incorporated herein by reference) by substituting human complementarity determining regions with corresponding rodent complementarity determining regions. Accordingly, such humanised antibodies are chimeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies. Chimeric antibodies are discussed by Neuberger et al (1998, 8^{th} International Biotechnology Symposium Part 2, 792-799).

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95, the disclosures of which are incorporated herein by reference).

It will be appreciated by persons skilled in the art that humanised antibodies or antigen-binding fragments that specifically bind to CD137 may further comprise a heavy chain constant region, or part thereof (see below).

In one embodiment, the antibody or antigen-binding fragment thereof that specifically binds to CD137 comprises a CH1, CH2 and/or CH3 region of an IgG heavy chain (such as an IgG1, IgG2, IgG3 or IgG4 heavy chain). Thus, the antibody or antigen-binding fragment thereof that specifically binds to CD137 may comprise part or all of the constant regions from an IgG4 heavy chain. For example, the antibody or antigen-binding fragment thereof that specifically binds to CD137 may be a Fab fragment comprising CH1 and CL constant regions, combined with any of the above-defined heavy and light variable regions respectively.

Likewise, the above-defined antibodies or antigen-binding fragments that specifically bind to CD137 may further comprise a light chain constant region, or part thereof (see below). For example, the antibody polypeptide may comprise a CL region from a kappa or lambda light chain.

In one embodiment, the antibodies or antigen-binding fragments that specifically bind to CD137 and are comprised in the combination therapy of the invention comprise an antibody Fc-region. It will be appreciated by a skilled person that the Fc portion may be from an IgG antibody, or from a different class of antibody (such as IgM, IgA, IgD or IgE). In one embodiment, the Fc region is from an IgG1, IgG2, IgG3 or IgG4 antibody. Advantageously, however, the Fc region is from an IgG4 antibody.

The Fc region may be naturally-occurring (e.g. part of an endogenously produced antibody) or may be artificial (e.g. comprising one or more point mutations relative to a naturally-occurring Fc region). A variant of an Fc region typically binds to Fc receptors, such as FcγR and/or neonatal Fc receptor (FcRn) with altered affinity providing for improved function and/or half-life of the polypeptide. The biological function and/or the half-life may be either increased or a decreased relative to the half-life of a polypeptide comprising a native Fc region. Examples of such biological functions which may be modulated by the presence of a variant Fc region include antibody dependent cell cytotoxicity (ADCC), antibody-dependent cellular phagocytosis (ADCP), complement-dependent cytotoxicity (CDC), and/or apoptosis.

Thus, the Fc region may be naturally-occurring (e.g. part of an endogenously produced human antibody) or may be artificial (e.g. comprising one or more point mutations relative to a naturally-occurring human Fc region).

As is well documented in the art, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP).

Engineering the Fc region of a therapeutic monoclonal antibody or Fc fusion protein allows the generation of molecules that are better suited to the pharmacology activity required of them (Strohl, 2009, Curr Opin Biotechnol 20(6):685-91, the disclosures of which are incorporated herein by reference).

### (a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher half-life in a variety of animal models.

Several mutations located at the interface between the CH2 and CH3 domains, such as T250Q/M428L (Hinton et al., 2004, 3 Biol Chem. 279(8):6213-6, the disclosures of which are incorporated herein by reference) and M252Y/S254T/T256E+H433K/N434F (Vaccaro et al., 2005, Nat. Biotechnol. 23(10):1283-8, the disclosures of which are incorporated herein by reference), have been shown to increase the binding affinity to FcRn and the half-life of IgG1 in vivo.

### (b) Engineered Fc Regions for Altered Effector Function

Depending on the therapeutic antibody or Fc fusion protein application, it may be desired to either reduce or increase the effector function (such as ADCC).

For antibodies that target cell-surface molecules, especially those on immune cells, abrogating effector functions may be required for certain clinical indications.

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, Blood. 113(16):3716-25, the disclosures of which are incorporated herein by reference).

### FcγRI Binding Affinity of IgG4 vs IgG2

Bruhns et al performed a series of experiments that evaluated the specificity and affinity of the known human FcγRs, and their polymorphic variants, for the different human IgG subclasses (Bruhns et al., 2009, Blood. 113(16):3716-25, the disclosures of which are incorporated herein by reference). In this study, it was clearly demonstrated that while IgG2 had no detectable affinity for FcγRI, IgG1, IgG3 and IgG4 all displayed a binding affinity for FcγRI in the nanomolar range (Bruhns et al., 2009, Blood. 113(16):3716-25, Lu et al., 2015, Proc Natl Acad Sci USA. 112(3):833-8, the disclosures of which are incorporated herein by reference). A summary of the relative binding affinities between the major human FcγRs and their variants and IgG isotypes is summarized in Table 1. (Stewart et al. 2014, J Immunother. 2(29), the disclosures of which are incorporated herein by reference)

TABLE 1
Binding affinity between human FcγRs and IgG isotypes.

| FCγR | IgG1 | IgG2 | IgG4 |
|---|---|---|---|
| FcγRI | ++++ | − | ++++ |
| FcγRIIA H131 | +++ | ++ | ++ |
| FcγRIIA R131 | +++ | + | ++ |
| FcγRIIB | ++ | − | ++ |
| FcγRIIIA V158 | +++ | + | ++ |
| FcγRIIIA F158 | ++ | − | ++ |

However, cellular activation influences the affinity of FcγRI for IgG immune complexes and the data generated by surface plasmon resonance in the Bruhns paper may not correctly reproduce what occurs at an inflammatory site. A review paper by Hogarth et al (Hogarth et al. 2012, Nat Rev Drug Discov 11(4):311-31, the disclosures of which are incorporated herein by reference) summarizes this as well as other studies focusing on FcγR binding for IgG.

### FcγRI Expression on Myeloid Cell Subsets

Human FcγRs are primarily expressed by cells of the myeloid lineage, which has been demonstrated in numerous studies for circulating myeloid cell subsets. Classical monocytes, generally identified as CD14⁺ CD16⁻ display high levels of FcγRII (CD32), intermediate levels of FcγRI and low levels of FcγRIII (CD16) (Almeida et al. 2001, 100(3):325-38, Cheeseman et al. 2016, PLoS One 11(5):e0154656, the disclosures of which are incorporated herein by reference). CD14⁻ CD16⁺ non-classical monocytes, however, display high levels of FcγRIII, intermediate levels of FcγRII and low levels of FcγRI (Almeida et al. 2001). A summary and compilation of several published microarray data sets showing the expression of human FcγR genes on different myeloid cell subsets confirms these observations (Guilliams et al. 2014, Nat Rev Immunol. 14(2):94-108, the disclosures of which are incorporated herein by reference).

Once within tissues, monocytes differentiate towards macrophages and, depending on environmental cues, these macrophages obtain specific phenotypes. In a study by Roussel et al (Roussel et al. 2017, J Leukoc Biol. 102(2):437-447, the disclosures of which are incorporated herein by reference), peripheral blood monocytes were polarized towards different macrophage lineages by using various inflammatory stimuli and the expression profile of these cells evaluated. Here, IFN-γ stimulated monocytes resulted in a highly elevated expression specifically of CD64. A similar observation was made in SLE patients where increased CD64 expression was detected on circulating CD14⁺ monocytes, which correlated with expression of interferon-stimulated genes (Li et al. 2010, Arthritis Res Ther 12(3): R90, the disclosures of which are incorporated herein by reference).

Myeloid Cell Infiltration within Various Human Tumours

Various myeloid cell subsets such as inflammatory monocytes, monocytic myeloid-derived suppressor cells (MDSC) and macrophages have, in numerous studies, been shown to accumulate in cancer patients (Solito et al. 2014, Ann N Y Acad Sci 1319:47-65., Hu et al. 2016, Clin Transl Oncol. 18(3):251-8, the disclosures of which are incorporated herein by reference). Although recent attempts have aimed at proposing strategies to standardize the characterization of these cells (Bronte et al. 2016, Nat Commun. 7:12150, the disclosures of which are incorporated herein by reference), many phenotypic definitions of these cell populations can still be found throughout the literature (Elliott et al. 2017, Front Immunol. 8:86, the disclosures of which are incorporated herein by reference). Most commonly, these cells are defined by the expression of the markers CD11b, CD14, CD33 and the low expression of HLA-DR (monocytic MDSC) (Bronte et al. 2016). Additionally, tumor-associated macrophages (TAM) are commonly identified by the expression of CD64 and CD68 (M1-polarized, anti-tumorigenic), or CD163 and CD206 (M2-polarized, pro-tumorigenic) (Elliott et al. 2017).

A recent review by Elliott et al summarizes the numerous phenotypes used to identify myeloid cell subsets in cancer patients. Most of these studies have focused their analyses on circulating cells and increased frequencies of myeloid CD11b⁺ cells have been observed in the blood of patients with e.g. bladder, breast, colorectal, hepatocellular, pancreatic, prostate and renal cell carcinoma (Solito et al. 2014, Elliott et al. 2017). Other studies have also attempted to characterize the level of infiltration of these cells into tumor tissue. In colorectal tumors, a high frequency of CD14⁺ CD169⁺ cells was observed. These cells also expressed CD163 and CD206 and were thus suggested to be M2-polarized TAM (Li et al. 2015, PLoS One 10(10):e0141817, the disclosures of which are incorporated herein by reference). Another study in colorectal cancer patients also detected increased numbers of CD11b⁺ CD33⁺ HLA-DR⁻ cells, compared to healthy individuals (Zhang et al. 2013, PLoS One 8(2):e57114, the disclosures of which are incorporated herein by reference).

Similarly, CD11b⁺ myeloid cells were also identified in bladder tumors, where they accounted for 10-20% of all nucleated cells (Eruslanov et al. 2012, Int J Cancer 130(5):1109-19, the disclosures of which are incorporated herein by reference). An even higher frequency of CD11b⁺ cells was observed in pancreatic cancer where over 60% of the CD45⁺ cells were CD11b⁺ CD15⁺ CD33⁺ (Porembka et al. 2012, Cancer Immunol Immunother 61(9):1373-85, the disclosures of which are incorporated herein by reference). Also, one study concluded that the macajor myeloid cell population within non-small cell lung carcinoma is a CD11b⁺ CD15⁺ CD66b⁺ neutrophil-like population. Interestingly, once these cells migrate from blood to the tumor tissue, these cells display an altered expression profile, including upregulated FcγRI (Eruslanov et al. 2014, J Clin Invest. 124(12):5466-80, the disclosures of which are incorporated herein by reference).

### FcγRI Expression on Tumor-Infiltrating Cells

Although numerous studies have identified a high infiltration of myeloid cells within human tumors, no study has thoroughly explored the expression of FcγRs on these cells in detail. Several publications have, however, demonstrated the presence of FcγRI-expressing cells within tumor tissue.

A study by Morimura et al (Morimura et al. 1990, Acta Neuropathol. 80(3):287-94, the disclosures of which are incorporated herein by reference) evaluated gliomas from 12 human samples by immunocytochemistry and compared these to peritumoral control tissue. This study demonstrated a high presence of macrophages (using the marker CD163, RM3/1) in gliomas, compared to peritumoral tissue, as well as an increase in FcγRI and FcγRII (CD32). A more recent study by Griesinger et al (Griesinger et al. 2013, J Immunol. 191(9):4880-8, the disclosures of which are incorporated herein by reference) confirmed these observations by performing flow cytometric analyses of various pediatric brain tumor types. Here, a high frequency of CD45⁺ CD11b⁺ myeloid cells was observed for tissues from pilocytic astrocytoma and ependymoma patients. These cells also expressed high levels of FcγRI.

In addition to brain tumors, FcγRI expression has also been shown for other types of tumors. Grugan et al (Grugan et al. 2012, J Immunol. 189(11):5457-66, the disclosures of which are incorporated herein by reference) demonstrated the presence of CD11b⁺ CD14⁺ cells within human breast tumor tissue. These cells were shown to express high levels of FcγRI and FcγRIIa, as well as FcγRIIb and FcγRIII. Also, CD45⁺ CD11b⁺ CD14⁺ CD68⁺ TAM were identified in gastrointestinal stromal tumors displaying expression of FcγRI (Cavnar et al. 2013, J Exp Med. 210(13):2873-86, the disclosures of which are incorporated herein by reference). CD45⁺ CD11b⁺ FcγRI⁺ cells were also identified in colorectal cancer patients and these cells displayed a higher expression of FcγRI in tumor tissue, compared to healthy control tissue (Norton et al. 2016, Clin Transl Immunology. 5(4):e76, the disclosures of which are incorporated herein by reference). FcγRI expression has also been demonstrated for melanoma metastases (Hansen et al. 2006, Acta Oncol 45(4):400-5, the disclosures of which are incorporated herein by reference).

Binding of IgG to the FcγRs or C1q depends on residues located in the hinge region and the CH2 domain. Two regions of the CH2 domain are critical for FcγRs and C1q binding, and have unique sequences in IgG2 and IgG4. Substitutions into human IgG1 of IgG2 residues at positions 233-236 and IgG4 residues at positions 327, 330 and 331 were shown to greatly reduce ADCC and CDC (Armour et al., 1999, Eur J Immunol. 29(8):2613-24; Shields et al., 2001, J Biol Chem. 276(9):6591-604, the disclosures of which are incorporated herein by reference). Furthermore, Idusogie et al. demonstrated that alanine substitution at different positions, including K322, significantly reduced complement activation (Idusogie et al., 2000, J Immunol. 164(8):4178-84, the disclosures of which are incorporated herein by reference). Similarly, mutations in the CH2 domain of murine IgG2A were shown to reduce the binding to FcγRI, and C1q (Steurer. et al., 1995. J Immunol. 155(3):1165-74, the disclosures of which are incorporated herein by reference).

Numerous mutations have been made in the CH2 domain of human IgG1 and their effect on ADCC and CDC tested in vitro (see references cited above). Notably, alanine substitution at position 333 was reported to increase both ADCC and CDC (Shields et al., 2001, supra; Steurer et al., 1995, supra). Lazar et al. described a triple mutant (S239D/I332E/A330L) with a higher affinity for FcγRIIIa and a lower affinity for FcγRIIb resulting in enhanced ADCC (Lazar et al., 2006, PNAS 103(11):4005-4010, the disclosures of which are incorporated herein by reference). The same mutations were used to generate an antibody with increased ADCC (Ryan et al., 2007, Mol. Cancer Ther. 6:3009-3018, the disclosures of which are incorporated herein by reference). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards et al., 2008. Mol Cancer Ther. 7(8):2517-27, the disclosures of which are incorporated herein by reference).

Due to their lack of effector functions, IgG4 antibodies represent a preferred IgG subclass for receptor modulation without cell depletion. IgG4 molecules can exchange half-molecules in a dynamic process termed Fab-arm exchange. This phenomenon can also occur in vivo between therapeutic antibodies and endogenous IgG4.

The S228P mutation has been shown to prevent this recombination process allowing the design of less unpredictable therapeutic IgG4 antibodies (Labrijn et al., 2009, Nat Biotechnol. 27(8):767-71, the disclosures of which are incorporated herein by reference).

In a further embodiment, the effector function of the Fc region may be altered through modification of the carbohydrate moieties within the CH2 domain therein, for example by modifying the relative levels of fucose, galactose, bisecting N-acetylglucosamine and/or sialic acid during production (see Jefferis, 2009, Nat Rev Drug Discov. 8(3):226-34 and Raju, 2008, Curr Opin Immunol., 20(4):471-8; the disclosures of which are incorporated herein by reference)

Thus, it is known that therapeutic antibodies lacking or low in fucose residues in the Fc region may exhibit enhanced ADCC activity in humans (for example, see Peipp et al., 2008, Blood 112(6):2390-9, Yamane-Ohnuki & Satoh, 2009, MAbs 1(3):230-26, Ilda et al., 2009, BMC Cancer 9; 58 (the disclosures of which are incorporated herein by reference). Low fucose antibody polypeptides may be produced by expression in cells cultured in a medium containing an inhibitor of mannosidase, such as kinfunensine (see Example I below).

Other methods to modify glycosylation of an antibody into a low fucose format include the use of the bacterial enzyme GDP-6-deoxy-D-lyxo-4-hexulose reductase in cells not able to metabolise rhamnose (e.g. using the GlymaxX® technology of ProBioGen AG, Berlin, Germany).

Another method to create low fucose antibodies is by inhibition or depletion of alpha-(1,6)-fucosyltransferase in the antibody-producing cells (e.g. using the Potelligent® CHOK1SV technology of Lonza Ltd, Basel, Switzerland).

An exemplary heavy chain constant region amino acid sequence which may be combined with any VH region sequence disclosed herein (to form a complete heavy chain) is the IgG1 heavy chain constant region sequence reproduced here:

[SEQ ID NO: 12]
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

Other heavy chain constant region sequences are known in the art and could also be combined with any VH region disclosed herein. For example, as indicated above, a preferred constant region is a modified IgG4 constant region such as that reproduced here:

[SEQ ID NO: 13]
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

This modified IgG4 sequence results in stabilization of the core hinge of IgG4 making the IgG4 more stable, preventing Fab arm exchange.

Another preferred constant region is a modified IgG4 constant region such as that reproduced here:

[SEQ ID NO: 14]
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNRYTQKSLSLSLGK

This modified IgG4 sequence exhibits reduced FcRn binding and hence results in a reduced serum half-life relative to wild type IgG4. In addition, it exhibits stabilization of the core hinge of IgG4 making the IgG4 more stable, preventing Fab arm exchange.

Also suitable for use in the polypeptides of the invention is a wild type IgG4 constant region such as that reproduced here:

[SEQ ID NO: 15]
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

An exemplary light chain constant region amino acid sequence which may be combined with any VL region sequence disclosed herein (to form a complete light chain) is the kappa chain constant region sequence reproduced here:

[SEQ ID NO: 16]
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Other light chain constant region sequences are known in the art and could also be combined with any VL region disclosed herein.

In an exemplary embodiment of the invention, the antibody or antigen binding fragment that specifically binds to CD137 may comprise the IgG4 constant regions of SEQ ID NOs: 13 and 16, respectively.

Thus, exemplary antibody or antigen-binding fragment thereof that specifically binds to CD137 may comprise:
(a) a heavy chain comprising a variable region of SEQ ID NO: 1 together with a constant region of SEQ ID NO: 13; and
(b) a light chain comprising a variable region of SEQ ID NO: 2 together with a constant region of SEQ ID NO: 16.

For example, the antibody or antigen-binding fragment thereof that specifically binds to CD137 may be an intact IgG4 molecule comprising or consisting of two heavy chains having an amino acid sequence of SEQ ID NO: 17 and two light chains having an amino acid sequence of SEQ ID NO: 18.

Alternative exemplary antibodies or antigen-binding fragments that specifically bind to CD137 may comprise:
(a) a heavy chain comprising a variable region of SEQ ID NO: 19 together with a constant region of SEQ ID NO: 13; and
(b) a light chain comprising a variable region of SEQ ID NO: 20 together with a constant region of SEQ ID NO: 16.

For example, the antibody or antigen-binding fragment thereof that specifically binds to CD137 may be an intact IgG4 molecule comprising or consisting of two heavy chains having an amino acid sequence of SEQ ID NO: 29 and two light chains having an amino acid sequence of SEQ ID NO: 30.

In one embodiment, the antibody or antigen-binding fragment thereof that specifically binds to CD137 is or comprises a “fusion” polypeptide.

In addition to being fused to a moiety in order to improve pharmacokinetic properties, it will be appreciated that the antibody or antigen-binding fragment thereof that specifically binds to CD137 that forms part of the combination therapy of the invention may also be fused to a polypeptide such as glutathione-S-transferase (GST) or protein A in order to facilitate purification of said polypeptide. Examples of such fusions are well known to those skilled in the art. Similarly, the antibody or antigen-binding fragment thereof that specifically binds to CD137 may be fused to an oligo-histidine tag, such as His6, or to an epitope recognised by an antibody such as the well-known Myc tag epitope. Fusions to any variant or derivative of said antibody or antigen-binding fragment thereof are also included in the scope of the invention. It will be appreciated that fusions (or variants, derivatives or fusions thereof) which retain or improve desirable properties, such as IL-1R binding properties or in vivo half-life are preferred.

Thus, the fusion may comprise an amino acid sequence as detailed above together with a further portion which confers a desirable feature on the said polypeptide comprised in the combination therapy of the invention; for example, the portion may useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the polypeptide, as known to those skilled in the art.

It will be appreciated by persons skilled in the art that the antibody or antigen-binding fragment thereof that specifically binds to CD137 comprised in the combination therapy of the invention may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

It will be further appreciated by persons skilled in the art that peptidomimetic compounds may also be useful. The term ‘peptidomimetic’ refers to a compound that mimics the conformation and desirable features of a particular peptide as a therapeutic agent.

For example, the antibody or antigen-binding fragment thereof that specifically binds to CD137 may include not only molecules in which amino acid residues are joined by peptide (—CO—NH—) linkages but also molecules in which the peptide bond is reversed.

Such retro-inverso peptidomimetics may be made using methods known in the art, for example such as those described in Meziere et al. (1997) J. Immunol. 159, 3230-3237, which is incorporated herein by reference. This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will also be appreciated that the said antibody or antigen-binding fragment thereof may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell et al., 1983, Biochem. Biophys. Res. Comm. 111:166, which are incorporated herein by reference.

Typically, the antibody or antigen-binding fragment thereof that specifically binds to CD137 will be a ‘naked’ antibody polypeptide, i.e. without any additional functional moieties such as cytotoxic or detectable moieties. For example, where the therapeutic effect is mediated by a direct effect of the antibody comprised in the combination therapy of the invention on immune cells, e.g. to reduce inflammation, it may be advantageous for the antibody to lack any cytotoxic activity.

However, in alternative embodiment, the antibody or antigen-binding fragment thereof that specifically binds to CD137 may be augmented with a functional moiety to facilitate their intended use, for example as a diagnostic (e.g. in vivo imaging) agent or therapeutic agent. Thus, in one embodiment, the antibody or antigen-binding fragment thereof that specifically binds to CD137 is linked, directly or indirectly, to a therapeutic moiety. A suitable therapeutic moiety is one that is capable of reducing or inhibiting the growth, or in particular killing, a cancer cell (or associated stem cells or progenitor cells). For example, the therapeutic agent may be a cytotoxic moiety, such as a radioisotope (e.g. ⁹⁰Y, ¹⁷⁷Lu, ⁹⁹Tc^{m}, etc) or cytotoxic drug (e.g. antimetabolites, toxins, cytostatic drugs, etc).

Alternatively, the cytotoxic moiety may comprise or consist of one or more moieties suitable for use in activation therapy, such as photon activation therapy, neutron activation therapy, neutron-induced Auger electron therapy, synchrotron irradiation therapy or low energy X-ray photon activation therapy.

Optionally, the antibody or antigen-binding fragment thereof that specifically binds to CD137 may further comprise a detectable moiety. For example, a detectable moiety may comprise or consist of a radioisotope, such as a radioisotope selected from the group consisting of ^{99m}Tc, ¹¹¹In, ⁶⁷Ga, ⁶⁸Ga, ⁷²As, ⁸⁹Zr, ¹²³I and ²⁰¹Tl Optionally, the agent may comprise a pair of detectable and cytotoxic radionuclides, such as ⁸⁶Y/⁹⁰Y or ¹²⁴I/²¹¹At. Alternatively, the antibody or antigen-binding fragment thereof that specifically binds to CD137 may comprise a radioisotope that is capable of simultaneously acting in a multi-modal manner as a detectable moiety and also as a cytotoxic moiety to provide so-called “Multimodality theragnostics”. The binding moieties may thus be coupled to nanoparticles that have the capability of multi-imaging (for example, SPECT, PET, MRI, Optical, or Ultrasound) together with therapeutic capability using cytotoxic drugs, such as radionuclides or chemotherapy agents.

Therapeutic and/or detectable moieties (such as a radioisotope, cytotoxic moiety or the like) may be linked directly, or indirectly, to the antibody or fragment thereof. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), derivatives of 3,6,9,15-Tetraazabicyclo[9.3.1]-pentadeca-1(15),11,13-triene-4-(S)-(4-isothiocyanato-benzyl)-3,6,9-triacetic acid (PCTA), derivatives of 5-S-(4-Aminobenzyl)-1-oxa-4,7,10-triazacyclododecane-4,7,10-tris(acetic acid) (DO3A) and other chelating moieties.

One preferred linker is DTPA, for example as used in ¹⁷⁷Lu-DTPA-[antibody polypeptide]. A further preferred linker is deferoxamine, DFO, for example as used in ⁸⁹Zr-DFO-[antibody polypeptide].

However, it will be appreciated by persons skilled in the art that many medical uses of the combination therapy of the invention comprising an antibody or antigen-binding fragment thereof that specifically binds to CD137 will not require the presence of a cytotoxic or diagnostic moiety.

As discussed above, methods for the production of antibody polypeptides that are comprised in the combination therapy of the invention are well known in the art.

Conveniently, the antibody or antigen-binding fragment thereof that specifically binds to CD137 is or comprises a recombinant polypeptide. Suitable methods for the production of such recombinant polypeptides are well known in the art, such as expression in prokaryotic or eukaryotic hosts cells (for example, see Green & Sambrook, 2012, Molecular Cloning, A Laboratory Manual, Fourth Edition, Cold Spring Harbor, New York, the relevant disclosures in which document are hereby incorporated by reference).

Although the antibody that specifically binds to CD137 may be a polyclonal antibody, it is preferred if it is a monoclonal antibody, or that the antigen-binding fragment, variant, fusion or derivative thereof, is derived from a monoclonal antibody.

Suitable monoclonal antibodies may be prepared by known techniques, for example those disclosed in “Monoclonal Antibodies; A manual of techniques”, H Zola (CRC Press, 1988) and in “Monoclonal Hybridoma Antibodies: Techniques and Application”, SGR Hurrell (CRC Press, 1982). Polyclonal antibodies may be produced which are poly-specific or mono-specific. It is preferred that they are mono-specific.

Antibody polypeptides comprised in the combination therapy of the invention can also be produced using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

It will be appreciated by persons skilled in the art that antibody or antigen-binding fragment thereof that specifically binds to CD137 may alternatively be synthesised artificially, for example using well known liquid-phase or solid phase synthesis techniques (such as t-Boc or Fmoc solid-phase peptide synthesis).

### (b) Further Immunotherapeutic Agent (PD-1 Inhibitor)

The combination therapies of the invention comprise a further immunotherapeutic agent, wherein the further immunotherapeutic agent is a PD-1 inhibitor. The PD-1 inhibitor may be effective in the treatment of cancer and/or may specifically bind to PD-1 or PD-L1. It will be appreciated that the therapeutic benefit of the further immunotherapeutic agent may be mediated by attenuating the function of the inhibitory immune checkpoint molecule PD-1.

Thus, in an embodiment of the invention, the PD-1 inhibitor is an immunotherapeutic agent with efficacy in the treatment of cancer.

The term “immunotherapeutic agent” is intended to include any molecule, peptide, antibody or other agent which can stimulate a host immune system to generate an immune response to a tumour or cancer in the subject. Various immunotherapeutic agents are useful in the compositions and methods described herein. In one embodiment, the immunotherapeutic agent is an antibody or antigen-binding fragment thereof, such as an anti-PD-1 antibody that is capable of specifically binding PD-1 or an anti-PD-L1 antibody which is capable of specifically binding PD-L1.

The term “immune response” includes T cell mediated and/or B cell mediated immune responses. Exemplary immune responses include T cell responses, e.g., cytokine production and cellular cytotoxicity. In addition, the term immune response includes immune responses that arc indirectly effected by T cell activation, e.g., antibody production (humoral responses) and activation of cytokine responsive cells, e.g., macrophages.

Immune checkpoint molecules include a group of proteins on the cell surface of immune cells, such as CD4+ and/or CD8+ T cells, dendritic cells, NK cells and macrophages but also on certain tumor cells, that modulate immune responses. It will be appreciated by persons skilled in the art that PD-1 is an inhibitory immune check point molecule.

Blocking or neutralisation of inhibitory immune checkpoint molecules, such as PD-1, can block or otherwise neutralise inhibitory signalling to thereby upregulate an immune response in order to more efficaciously treat cancer. Exemplary agents useful for blocking inhibitory immune checkpoint include antibodies, small molecules, peptides, peptidomimetics, natural ligands, and derivatives of natural ligands, that can either bind and/or inactivate or inhibit inhibitory immune checkpoint proteins, or fragments thereof; as well as RNA interference, antisense, nucleic acid aptamers, etc. that can downregulate the expression and/or activity of inhibitory immune checkpoint nucleic acids, or fragments thereof. Exemplary agents for upregulating an immune response include antibodies against one or more inhibitory immune checkpoint proteins that blocks the interaction between the proteins and its natural receptor(s); a non-activating form of one or more immune checkpoint inhibitor proteins {e.g., a dominant negative polypeptide): small molecules or peptides that block the interaction between one or more inhibitory immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block inhibitory immune checkpoint nucleic acid transcription or translation; and the like. Such agents can directly block the interaction between the one or more inhibitory immune checkpoint and its natural receptor(s) (e.g., antibodies) to prevent inhibitory signalling and upregulate an immune response. Alternatively, agents can indirectly block the interaction between one or more inhibitory immune checkpoint proteins and its natural receptor(s) to prevent inhibitory signalling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies and/or anti-PD-L1 antibodies either alone or in combination, are used to inhibit immune checkpoint inhibitors.

Thus, in one embodiment, the further immunotherapeutic agent is a PD-1 inhibitor that binds to and inhibits the function of an inhibitory immune checkpoint molecule.

By “PD-1 inhibitor” (or “PD-1 pathway inhibitor”) we include an entity which is capable of inhibiting the PD-1 pathway.

PD-1 serves as a negative regulator of T cell activation when engaged with its ligands PD-L1 or PD-L2. PD-L1 in particular is expressed by many solid tumors, including melanoma. These tumours may therefore down regulate immune mediated anti-tumor effects through activation of the inhibitory PD-1 receptors on T cells. By blocking the interaction between PD-1 and PD-L1, a check point of the immune response may be removed, leading to augmented anti-tumour T cell responses. This interaction may be blocked by an antibody specific for PD-1 or PD-Lior any other suitable agent. Such antibodies and agents may be generally referred to as PD-1 inhibitors. The further immunotherapeutic agent in step (b) of the method of the invention is a PD-1 inhibitor.

Accordingly, PD-1 inhibitors block the interaction of PD-1 (programmed cell death protein 1) with its ligand PD-L1 (programmed death-ligand 1). Such PD-1 inhibitors can therefore act on either, or both, PD-L1 and PD-1. Thus, the term PD-1 inhibitors includes both PD-1 and PD-L1 inhibitors. PD-1 inhibitors block the activity of PD-1 and PD-L1 immune checkpoint proteins.

By “PD-1”, we specifically include the human PD-1 protein, for example as described in GenBank Accession No. NP_005009.2 (the sequence of which is set out in SEQ ID NO: 35, below). PD-1 is also know in the scientific literature as PD1, CD279, PDCD1 and SLEB2.

[SEQ ID NO: 35]
MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

By “PD-L1” we specifically include the human PD-L1 protein, for example as described in GenBank Accession No. AAI13735.1 (the sequence of which is set out in SEQ ID NO: 36, below). PD-L1 is also know in the scientific literature as CD274, B7-H1, B7-H, PDCD1L1 and PDCD1LG1

[SEQ ID NO: 36]
MRIFAVFIFM TYWHLLNAFT VTVPKDLYVV EYGSNMTIEC
KFPVEKQLDL AALIVYWEME DKNIIQFVHG EEDLKVQHSS
YRQRARLLKD QLSLGNAALQ ITDVKLQDAG VYRCMISYGG
ADYKRITVKV NAPYNKINQR ILVVDPVTSE HELTCQAEGY
PKAEVIWTSS DHQVLSGKTT TTNSKREEKL FNVTSTLRIN
TTTNEIFYCT FRRLDPEENH TAELVIPELP LAHPPNERTH
LVILGAILLC LGVALTFIFR LRKGRMMDVK KCGIQDTNSK
KQSDTHLEET

Thus, the combination therapy of the invention comprises a PD-1 inhibitor that specifically binds to PD-1 or PD-L1 i.e. has specificity for PD-1 or PD-L1. By “specificity” we mean that the inhibitor e is capable of binding to PD-1 or PD-L1 in vivo, i.e. under the physiological conditions in which PD-1 or PD-L1 exists within the human body. Preferably, the PD-1 inhibitor does not bind to any other protein (other than PD-1 or PD-L1) in vivo. Such binding specificity may be determined by methods well known in the art, such as ELISA, immunohistochemistry, immunoprecipitation, Western blots and flow cytometry using transfected cells expressing PD-1 or PD-L1.

The PD-1 inhibitor that specifically binds to PD-1 or PD-L1 preferably binds to human PD-1 or PD-L1 with a Kd value which is less than 10×10⁻⁹M or less than 7×10⁻⁹M, more preferably less than 4, or 2×10⁻⁹M, most preferably less than 1.2×10⁻⁹M. Advantageously, the PD-1 inhibitor is capable of binding selectively to PD-1 or PD-L1, i.e. it bind at least 10-fold more strongly to PD-1 or PD-L1 than to any other proteins. The PD-1 inhibitor preferably specifically binds to PD-1 or PD-L1, i.e. it binds to PD-1 or PD-L1 but does not bind, or binds at a lower affinity (e.g. a 10-fold lower affinity), to other molecules (such as OX40 and/or CD40)—it therefore binds to PD-1 or PD-L1 with greater binding affinity than that at which it binds another molecule. Therefore, typically, the Kd for the antibody with respect to human PD-1 or PD-L1 will be 2-fold, preferably 5-fold, more preferably 10-fold less than Kd with respect to the other, non-target molecule, such as murine PD-1 or PD-L1, other immune checkpoint molecules, or any other unrelated material or accompanying material in the environment. More preferably, the Kd will be 50-fold less, even more preferably 100-fold less, and yet more preferably 200-fold less.

Methods for measuring the overall affinity (KD) and on-rate (ka) and off-rate (kd) of an interaction (such as an interaction between an antibody and a ligand) are well known in the art. Exemplary in vitro methods are described in the accompanying Examples. It is also conceivable to use flow cytometry based methods (Sklar et al., Annu Rev Biophys Biomol Struct, (31), 97-119, 2002).

The terms PD-1 and PD-L1 as used herein typically refers to human PD-1 and PD-L1. The inhibitor may have some binding affinity for PD-1 or PD-L1 from other mammals, such as PD-1 or PD-L1 from a non-human primate, for example Macaca fascicularis (cynomolgus monkey). The antibody preferably does not bind to murine PD-1 or PD-L1 and/or does not bind to other immune checkpoint molecules.

In an embodiment, the PD-1 inhibitor thereof that specifically binds to PD-1 or PD-L1 may have affinity for PD-1 or PD-L1 in its native state, for example for PD-1 or PD-L1 localised on the surface of a cell. In an embodiment, the PD-1 inhibitor blocks the PD-1 PD-L1 interaction. For example, the PD-1 inhibitor may bind to PD-1 or PD-L1 in a manner that inhibits the ability of PD-L1 to bind to PD-1, thereby blocking the PD-1/PD-L1 interaction.

By “localised on the surface of a cell” it is meant that PD-1 or PD-L1 is associated with the cell such that one or more region of PD-1 is present on the outer face of the cell surface. For example, PD-1 may be inserted into the cell plasma membrane (i.e. orientated as a transmembrane protein) with one or more regions presented on the extracellular surface. This may occur in the course of expression of PD-1 by the cell. Thus, in one embodiment, “localised on the surface of a cell” may mean “expressed on the surface of a cell.” Alternatively, PD-1 may be outside the cell with covalent and/or ionic interactions localising it to a specific region or regions of the cell surface.

In an embodiment, the PD-1 inhibitors described here are capable of inducing antitumour immunity, via immune checkpoint blockade. The PD-1 inhibitor binds to PD-1 or PD-L1 in a manner that inhibits PD-L1 to bind to PD-1, i.e. blocks the PD-1/PD-L1 interaction. The PD-1 inhibitor may be capable of enhancing T cell responses, for example it may be capable of enhancing or restoring T cell effector function. In one embodiment the PD-1 inhibitor may promote infiltration of tumour reactive CD8+ T cells into established tumours.

Thus, PD-1 inhibitor may modulate the activity of a cell expressing PD-1 or PD-L1, wherein said modulation is an increase or decrease in the activity of said cell. The cell is typically a T cell. The inhibitor may increase the activity of a CD4+ or CD8+ effector cell, or may decrease the activity of, or deplete, a regulatory T cell (T reg). In either case, the net effect of the antibody will be an increase in the activity of effector T cells, particularly CD4+, CD8+ or NK effector T cells. Methods for determining a change in the activity of effector T cells are well known and are as described earlier.

The PD-1 inhibitor preferably causes an increase in activity in a T cell in vitro, preferable a CD8+ T cell, optionally wherein said increase in activity is an increase in proliferation, IFN-γ production and/or IL-2 production by the T cell. The increase is preferably at least 2-fold, more preferably at least 10-fold and even more preferably at least 25-fold higher than the change in activity caused by an isotype control antibody measured in the same assay.

In one embodiment, the PD-1 inhibitors are capable of improving efficacy of another immunotherapy.

In one embodiment, the PD-1 inhibitor blocks the programmed death-1 (PD-1) receptor to its ligand PD-L1, expressed on the surface of cells within a tumor (Ribas and Wolchok 2018). PD-1 is an immune checkpoint, with its inhibitory function mediated by the tyrosine phosphatase SHP-2 that de-phosphorylates signaling molecules downstream of the T cell receptor (TCR) signaling molecules. In a preferred embodiment, the PD-1 inhibitor reactivates PD-1 expressing T cells, preferably by blocking the inhibitory signaling mediated by the tyrosine phosphatase SHP-2 (that de-phosphorylates signaling molecules downstream of the T cell receptor (TCR) signaling molecules.

The PD-1 inhibitor may be an anti-PD-1 antibody, or antigen-binding fragment thereof capable of inhibiting PD-1 function (for example, Pembrolizumab (also known as Lambrolizumab), Nivolumab, Pidilizumab, Cemiplimab, AMP-224, PDR-001, MEDI-0680 (also known as AMP-514), JTX-4014 (Pimivalimab), Spartalizumab, Camrelizumab, Sintilimab, Tislelizumab, Toripalimab, Dostarlimab and INCMGA00012 (Retifanlimab).

Alternatively, the PD-1 inhibitor may comprise or consist of an anti-PD-L1 antibody, or antigen-binding fragment thereof capable of inhibiting PD-1 function (for example, Atezolizumab (Tecentriq™, MPDL3280A), Durvalumab (MEDI-4736), Avelumab, MDX-1105, KN035 (Envafolimab) and CK-301 (Cosibelimab)).

Alternatively the PD-1 inhibitor may be a small molecule or peptide based inhibitor of PD-1 or PD-L1. For example the PD-1 inhibitor may be a small molecule inhibitor of PD-L1 such as CA-170. Alternatively, the PD-1 inhibitor may be a peptide inhibitor of PD-L1 such as AUNP12 or BMS-986189.

In one embodiment, the PD-1 inhibitor binds to an epitope that blocks the PD-1 PD-L1 interaction.

In one embodiment, the PD-1 inhibitor is capable of inhibiting binding of reference antibodies Pembrolizumab or Nivolumab to human PD-1.

In another embodiment, the PD-1 inhibitor is capable of inhibiting binding of reference antibody Atezolizumab to human PD-L1.

By “capable of inhibiting the binding of reference antibody Pembrolizumab to human PD-1” we mean that the presence of the PD-1 inhibitor of the combination therapy of the invention inhibits, in whole or in part, the binding of Pembrolizumab to human PD-1. Similarly, by “capable of inhibiting the binding of reference antibody Nivolumab to human PD-1” we mean that the presence of the antibody polypeptides of the combination therapy of invention inhibits, in whole or in part, the binding of Nivolumab to human PD-1. The PD-1 inhibitors used in the combination therapies of the invention may therefore compete for binding to PD-1 with ‘reference antibody’ Pembrolizumab and/or with ‘reference antibody’ Nivolumab. Such competitive binding inhibition can be determined using assays and methods well known in the art, for example using BIAcore chips with immobilised PD-1 and incubating in the presence the reference antibody Pembrolizumab and Nivolumab with and without an antibody polypeptide to be tested. Alternatively, a pair-wise mapping approach can be used, in which the reference antibody Pembrolizumab or Nivolumab is immobilised to the surface of the BIAcore chip, PD-1 antigen is bound to the immobilised antibody, and then a second antibody is tested for simultaneous PD-1-binding ability (see ‘BIAcore Assay Handbook’, GE Healthcare Life Sciences, 29-0194-00 AA 05/2012; the disclosures of which are incorporated herein by reference).

By “capable of inhibiting the binding of reference antibody Atezolizumab to human PD-L1” we mean that the presence of the antibody polypeptides of the combination therapy of invention inhibits, in whole or in part, the binding of Atezolizumab to human PD-L1. The PD-1 inhibitors used in the combination therapies of the invention may therefore compete for binding to PD-L1 with ‘reference antibody’ Atezolizumab. Such competitive binding inhibition can be determined using assays and methods well known in the art, for example using BIAcore chips with immobilised PD-L1 and incubating in the presence the reference antibody Atezolizumab with and without an antibody polypeptide to be tested. Alternatively, a pair-wise mapping approach can be used, in which the reference antibody Atezolizumab is immobilised to the surface of the BIAcore chip, PD-L1 antigen is bound to the immobilised antibody, and then a second antibody is tested for simultaneous PD-L1-binding ability (see ‘BIAcore Assay Handbook’, GE Healthcare Life Sciences, 29-0194-00 AA 05/2012; the disclosures of which are incorporated herein by reference).

In a further alternative, competitive binding inhibition can be determined using flow cytometry. For example, to test whether a test antibody is able to inhibit the binding of the Pembrolizumab or Nivolumab (or Atezolizumab) reference antibody to a cell surface antigen, cells expressing the antigen can be pre-incubated with the test antibody for 20 min before cells are washed and incubated with the reference Pembrolizumab or Nivolumab (or Atezolizumab) antibody conjugated to a fluorophore, which can be detected by flow cytometry. If the pre-incubation with the test antibody reduces the detection of the reference Pembrolizumab or Nivolumab (or Atezolizumab) antibody in flow cytometry, the test antibody inhibits the binding of the reference antibody to the cell surface antigen. If the antibody to be tested exhibits high affinity for PD-1 (or PD-L1), then a reduced pre-incubation period may be used (or even no pre-incubation at all).

By “Pembrolizumab” we mean an intact IgG antibody comprising heavy and light chains having the amino acid sequences of SEQ ID NOS: 33 and 34, respectively.

Heavy chain sequence of Pembrolizumab
(SEQ ID NO: 33):
QVQLVQSGVE VKKPGASVKV SCKASGYTFT NYYMYWVRQA
PGQGLEWMGG INPSNGGTNF NEKFKNRVTL TTDSSTTTAY
MELKSLQFDD TAVYYCARRD YRFDMGFDYW GQGTTVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
Light chain sequence of Pembrolizumab
(SEQ ID NO: 34)
EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

By “Nivolumab” we mean an intact IgG antibody comprising heavy and light chains having the amino acid sequences of SEQ ID NOS: 31 and 32, respectively.

Heavy chain sequence of Nivolumab (SEQ ID NO: 31):
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS
VVTVPSSSLG TKTYTCNVDH KPSNTKVDKR VESKYGPPCP PCPAPEFLGG PSVFLFPPKP
KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGK
Light chain sequence of Nivolumab (SEQ ID NO: 32)
EIVLTQSPAT LSLSPGERAT LSCRASQSVS SYLAWYQQKP GQAPRLLIYD ASNRATGIPA
RFSGSGSGTD FTLTISSLEP EDFAVYYCQQ SSNWPRTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of Pidilizumab (SEQ ID NO: 37)
QVQLVQSGSE LKKPGASVKI SCKASGYTFT NYGMNWVRQA PGQGLQWMGW
INTDSGESTY AEEFKGRFVF SLDTSVNTAY LQITSLTAED TGMYFCVRVG YDALDYWGQG
TLVTVSSAST KGPSVFPLAP SSKSTSGGTA ALGCLVKDYF PEPVTVSWNS GALTSGVHTF
PAVLQSSGLY SLSSVVTVPS SSLGTQTYIC NVNHKPSNTK VDKRVEPKSC DKTHTCPPCP
APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT
LPPSREEMTK NQVSLTCLVK GFYPSDIAVE WESNGQPENN YKTTPPVLDS DGSFFLYSKL
TVDKSRWQQG NVFSCSVMHE ALHNHYTQKS LSLSPGK
Light chain sequence of Pidilizumab (SEQ ID NO: 38)
EIVLTQSPSS LSASVGDRVT ITCSARSSVS YMHWFQQKPG KAPKLWIYRT SNLASGVPSR
FSGSGSGTSY CLTINSLQPE DFATYYCQQR SSFPLTFGGG TKLEIKRTVA APSVFIFPPS
DEQLKSGTAS VVCLLNNFYP REAKVQWKVD NALQSGNSQE SVTEQDSKDS TYSLSSTLTL
SKADYEKHKV YACEVTHQGL SSPVTKSFNR GEC
Heavy chain sequence of Cemiplimab (SEQ ID NO: 39)
EVQLLESGGV LVQPGGSLRL SCAASGFTFS NFGMTWVRQA PGKGLEWVSG ISGGGRDTYF
ADSVKGRFTI SRDNSKNTLY LQMNSLKGED TAVYYCVKWG NIYFDYWGQG TLVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVF
SCSVMHEALH NHYTQKSLSL SLGK
Light chain sequence of Cemiplimab (SEQ ID NO: 40)
DIQMTQSPSS LSASVGDSIT ITCRASLSIN TFLNWYQQKP GKAPNLLIYA ASSLHGGVPS
RFSGSGSGTD FTLTIRTLQP EDFATYYCQQ SSNTPFTFGP GTVVDFRRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of Spartalizumab (SEQ ID NO: 41)
EVQLVQSGAE VKKPGESLRI SCKGSGYTFT TYWMHWVRQA TGQGLEWMGN IYPGTGGSNF
DEKFKNRVTI TADKSTSTAY MELSSLRSED TAVYYCTRWT TGTGAYWGQG TTVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVF
SCSVMHEALH NHYTQKSLSL SLG
Light chain sequence of Spartalizumab (SEQ ID NO: 42)
EIVLTQSPAT LSLSPGERAT LSCKSSQSLL DSGNQKNFLT WYQQKPGQAP RLLIYWASTR
ESGVPSRFSG SGSGTDFTFT ISSLEAEDAA TYYCQNDYSY PYTFGQGTKV EIKRTVAAPS
VFIFPPSDEQ LKSGTASVVC LLNNFYPREA KVQWKVDNAL QSGNSQESVT EQDSKDSTYS
LSSTLTLSKA DYEKHKVYAC EVTHQGLSSP VTKSFNRGEC
Heavy chain sequence of Camrelizumab (SEQ ID NO: 43)
EVQLVESGGG LVQPGGSLRL SCAASGFTFS SYMMSWVRQA PGKGLEWVAT ISGGGANTYY
PDSVKGRFTIS RDNAKNSLYL QMNSLRAEDT AVYYCARQLY YFDYWGQGTT VTVSSASTKG
PSVFPLAPCS RSTSESTAAL GCLVKDYFPE PVTVSWNSGA LTSGVHTFPA VLQSSGLYSL
SSVVTVPSSS LGTKTYTCNV DHKPSNTKVD KRVESKYGPP CPPCPAPEFL GGPSVFLFPP
KPKDTLMISR TPEVTCVVVD VSQEDPEVQF NWYVDGVEVH NAKTKPREEQ FNSTYRVVSV
LTVLHQDWLN GKEYKCKVSN KGLPSSIEKT ISKAKGQPRE PQVYTLPPSQ EEMTKNQVSL
TCLVKGFYPS DIAVEWESNG QPENNYKTTP PVLDSDGSFF LYSRLTVDKS RWQEGNVFSC
SVMHEALHNH YTQKSLSLSL GK
Light chain sequence of Camrelizumab (SEQ ID NO: 44)
DIQMTQSPSS LSASVGDRVT ITCLASQTIG TWLTWYQQKP GKAPKLLIYT ATSLADGVPS
RFSGSGSGTD FTLTISSLQP EDFATYYCQQ VYSIPWTFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of Tislelizumab (SEQ ID NO: 45)
QVQLQESGPG LVKPSETLSL TCTVSGFSLT SYGVHWIRQP PGKGLEWIGV IYADGSTNYN
PSLKSRVTIS KDTSKNQVSL KLSSVTAADT AVYYCARAYG NYWYIDVWGQ GTTVTVSSAS
TKGPSVFPLA PCSRSTSEST AALGCLVKDY FPEPVTVSWN SGALTSGVHT FPAVLQSSGL
YSLSSVVTVP SSSLGTKTYT CNVDHKPSNT KVDKRVESKY GPPCPPCPAP PVAGGPSVFL
FPPKPKDTLM ISRTPEVTCV VVAVSQEDPE VQFNWYVDGV EVHNAKTKPR EEQFNSTYRV
VSVLTVVHQD WLNGKEYKCK VSNKGLPSSI EKTISKAKGQ PREPQVYTLP PSQEEMTKNQ
VSLTCLVKGF YPSDIAVEWE SNGQPENNYK TTPPVLDSDG SFFLYSKLTV DKSRWQEGNV
FSCSVMHEAL HNHYTQKSLS LSLGK
Light chain sequence of Tislelizumab (SEQ ID NO: 46)
DIVMTQSPDS LAVSLGERAT INCKSSESVS NDVAWYQQKP GQPPKLLINY AFHRFTGVPD
RFSGSGYGTD FTLTISSLQA EDVAVYYCHQ AYSSPYTFGQ GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of Toripalimab (SEQ ID NO: 47)
QGQLVQSGAE VKKPGASVKV SCKASGYTFT DYEMHWVRQA PIHGLEWIGV IESETGGTAY
NQKFKGRVTI TADKSTSTAY MELSSLRSED TAVYYCAREG ITTVATTYYW YFDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVFSC SVMHEALHNH YTQKSLSLSL GK
Light chain sequence of Toripalimab (SEQ ID NO: 48)
DVVMTQSPLS LPVTLGQPAS ISCRSSQSIV HSNGNTYLEW YLQKPGQSPQ LLIYKVSNRF
SGVPDRFSGS GSGTDFTLKI SRVEAEDVGV YYCFQGSHVP LTFGQGTKLE IKRTVAAPSV
FIFPPSDEQL KSGTASVVCL LNNFYPREAK VQWKVDNALQ SGNSQESVTE QDSKDSTYSL
SSTLTLSKAD YEKHKVYACE VTHQGLSSPV TKSFNRGEC
Heavy chain sequence of Dostarlimab (SEQ ID NO: 49)
EVQLLESGGG LVQPGGSLRL SCAASGFTFS SYDMSWVRQA PGKGLEWVST ISGGGSYTYY
QDSVKGRFTI SRDNSKNTLY LQMNSLRAED TAVYYCASPY YAMDYWGQGT TVTVSSASTK
GPSVFPLAPC SRSTSESTAA LGCLVKDYFP EPVTVSWNSG ALTSGVHTFP AVLQSSGLYS
LSSVVTVPSS SLGTKTYTCN VDHKPSNTKV DKRVESKYGP PCPPCPAPEF LGGPSVFLFP
PKPKDTLMIS RTPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPS QEEMTKNQVS
LTCLVKGFYP SDIAVEWESN GQPENNYKTT PPVLDSDGSF FLYSRLTVDK SRWQEGNVFS
CSVMHEALHN HYTQKSLSLS LGK
Light chain sequence of Dostarlimab (SEQ ID NO: 50)
DIQLTQSPSF LSAYVGDRVT ITCKASQDVG TAVAWYQQKP GKAPKLLIYW ASTLHTGVPS
RFSGSGSGTE FTLTISSLQP EDFATYYCQH YSSYPWTFGQ GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of INCMGA00012 (SEQ ID NO: 51)
QVQLVQSGAE VKKPGASVKV SCKASGYSFT SYWMNWVRQA PGQGLEWIGV
IHPSDSETWL DQKFKDRVTI TVDKSTSTAY MELSSLRSED TAVYYCAREH YGTSPFAYWG
QGTLVTVSSA STKGPSVFPL APCSRSTSES TAALGCLVKD YFPEPVTVSW NSGALTSGVH
TFPAVLQSSG LYSLSSVVTV PSSSLGTKTY TCNVDHKPSN TKVDKRVESK YGPPCPPCPA
PEFLGGPSVF LFPPKPKDTL MISRTPEVTC VVVDVSQEDP EVQFNWYVDG VEVHNAKTKP
REEQFNSTYR VVSVLTVLHQ DWLNGKEYKC KVSNKGLPSS IEKTISKAKG QPREPQVYTL
PPSQEEMTKN QVSLTCLVKG FYPSDIAVEW ESNGQPENNY KTTPPVLDSD GSFFLYSRLT
VDKSRWQEGN VFSCSVMHEA LHNHYTQKSL SLSLG
Light chain sequence of INCMGA00012 (SEQ ID NO: 52)
EIVLTQSPAT LSLSPGERAT LSCRASESVD NYGMSFMNWF QQKPGQPPKL LIHAASNQGS
GVPSRFSGSG SGTDFTLTIS SLEPEDFAVY FCQQSKEVPY TFGGGTKVEI KRTVAAPSVF
IFPPSDEQLK SGTASVVCLL NNFYPREAKV QWKVDNALQS GNSQESVTEQ DSKDSTYSLS
STLTLSKADY EKHKVYACEV THQGLSSPVT KSFNRGEC
By “Atezolizumab” we mean an intact IgG antibody comprising heavy
and light chains having the amino acid sequences of SEQ ID NOS:
53 and 54, respectively.
Heavy chain sequence of Atezolizumab (SEQ ID NO: 53)
EVQLVESGGG LVQPGGSLRL SCAASGFTFS DSWIHWVRQA PGKGLEWVAW ISPYGGSTYY
ADSVKGRFTI SADTSKNTAY LQMNSLRAED TAVYYCARRH WPGGFDYWGQ GTLVTVSSAS
TKGPSVFPLA PSSKSTSGGT AALGCLVKDY FPEPVTVSWN SGALTSGVHT FPAVLQSSGL
YSLSSVVTVP SSSLGTQTYI CNVNHKPSNT KVDKKVEPKS CDKTHTCPPC PAPELLGGPS
VFLFPPKPKD TLMISRTPEV TCVVVDVSHE DPEVKFNWYV DGVEVHNAKT KPREEQYAST
YRVVSVLTVL HQDWLNGKEY KCKVSNKALP APIEKTISKA KGQPREPQVY TLPPSREEMT
KNQVSLTCLV KGFYPSDIAV EWESNGQPEN NYKTTPPVLD SDGSFFLYSK LTVDKSRWQQ
GNVFSCSVMH EALHNHYTQK SLSLSPGK
Light chain sequence of Atezolizumab (SEQ ID NO: 54)
DIQMTQSPSS LSASVGDRVT ITCRASQDVS TAVAWYQQKP GKAPKLLIYS ASFLYSGVPS
RFSGSGSGTD FTLTISSLQP EDFATYYCQQ YLYHPATFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Heavy chain sequence of Durvalumab (SEQ ID NO: 55)
EVQLVESGGG LVQPGGSLRL SCAASGFTFS RYWMSWVRQA PGKGLEWVAN IKQDGSEKYY
VDSVKGRFTI SRDNAKNSLY LQMNSLRAED TAVYYCAREG GWFGELAFDY WGQGTLVTVS
SASTKGPSVF PLAPSSKSTS GGTAALGCLV KDYFPEPVTV SWNSGALTSG VHTFPAVLQS
SGLYSLSSVV TVPSSSLGTQ TYICNVNHKP SNTKVDKRVE PKSCDKTHTC PPCPAPEFEG
GPSVFLFPPK PKDTLMISRT PEVTCVVVDV SHEDPEVKFN WYVDGVEVHN AKTKPREEQY
NSTYRVVSVL TVLHQDWLNG KEYKCKVSNK ALPASIEKTI SKAKGQPREP QVYTLPPSRE
EMTKNQVSLT CLVKGFYPSD IAVEWESNGQ PENNYKTTPP VLDSDGSFFL YSKLTVDKSR
WQQGNVFSCS VMHEALHNHY TQKSLSLSPG K
Light chain sequence of Durvalumab (SEQ ID NO: 56)
EIVLTQSPGT LSLSPGERAT LSCRASQRVS SSYLAWYQQK PGQAPRLLIY DASSRATGIP
DRFSGSGSGT DFTLTISRLE PEDFAVYYCQ QYGSLPWTFG QGTKVEIKRT VAAPSVFIFP
PSDEQLKSGT ASVVCLLNNF YPREAKVQWK VDNALQSGNS QESVTEQDSK DSTYSLSSTL
TLSKADYEKH KVYACEVTHQ GLSSPVTKSF NRGEC
Heavy chain sequence of Avelumab (SEQ ID NO: 57)
EVQLLESGGG LVQPGGSLRL SCAASGFTFS SYIMMWVRQA PGKGLEWVSS IYPSGGITFY
ADTVKGRFTI SRDNSKNTLY LQMNSLRAED TAVYYCARIK LGTVTTVDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
Light chain sequence of Avelumab (SEQ ID NO: 58)
QSALTQPASV SGSPGQSITI SCTGTSSDVG GYNYVSWYQQ HPGKAPKLMI YDVSNRPSGV
SNRFSGSKSG NTASLTISGL QAEDEADYYC SSYTSSSTRV FGTGTKVTVL GQPKANPTVT
LFPPSSEELQ ANKATLVCLI SDFYPGAVTV AWKADGSPVK AGVETTKPSK QSNNKYAASS
YLSLTPEQWK SHRSYSCQVT HEGSTVEKTV APTECS
Heavy chain sequence of CK-301 (Cosibelimab) (SEQ ID NO: 59)
EVQLVQSGAE VKKPGSSVKV SCKASGGTFS RSAISWVRQA PGQGLEWMGV IIPAFGEANY
AQKFQGRVTI TADESTSTAY MELSSLRSED TAVYYCARGR QMFGAGIDFW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP KSCDKTHTCP PCPAPELLGG
PSVFLFPPKP KDTLMISRTP EVTCVVVDVS HEDPEVKFNW YVDGVEVHNA KTKPREEQYN
STYRVVSVLT VLHQDWLNGK EYKCKVSNKA LPAPIEKTIS KAKGQPREPQ VYTLPPSRDE
LTKNQVSLTC LVKGFYPSDI AVEWESNGQP ENNYKTTPPV LDSDGSFFLY SKLTVDKSRW
QQGNVFSCSV MHEALHNHYT QKSLSLSPGK
Light chain sequence of CK-301 (Cosibelimab) (SEQ ID NO: 60)
NFMLTQPHSV SESPGKTVTI SCTRSSGSID SNYVQWYQQR PGSAPTTVIY EDNQRPSGVP
DRFSGSIDSS SNSASLTISG LKTEDEADYY CQSYDSNNRH VIFGGGTKLT VLGQPKAAPS
VTLFPPSSEE LQANKATLVC LISDFYPGAV TVAWKADSSP VKAGVETTTP SKQSNNKYAA
SSYLSLTPEQ WKSHRSYSCQ VTHEGSTVEK TVAPTECS
Heavy chain sequence of JTX-4014 (SEQ ID NO: 61)
QVQLVQSGAE VKKPGASVKV SCKASGYTFP SYYMHWVRQA PGQGLEWMGI INPEGGSTAY
AQKFQGRVTM TRDTSTSTVY MELSSLRSED TAVYYCARGG TYYDYTYWGQ GTLVTVSSAS
TKGPSVFPLA PCSRSTSEST AALGCLVKDY FPEPVTVSWN SGALTSGVHT FPAVLQSSGL
YSLSSVVTVP SSSLGTKTYT CNVDHKPSNT KVDKRVESKY GPPCPPCPAP EFLGGPSVFL
FPPKPKDTLM ISRTPEVTCV VVDVSQEDPE VQFNWYVDGV EVHNAKTKPR EEQFNSTYRV
VSVLTVLHQD WLNGKEYKCK VSNKGLPSSI EKTISKAKGQ PREPQVYTLP PSQEEMTKNQ
VSLTCLVKGF YPSDIAVEWE SNGQPENNYK TTPPVLDSDG SFFLYSRLTV DKSRWQEGNV
FSCSVMHEAL HNHYTQKSLS LSLGK
Light chain sequence of JTX-4014 (SEQ ID NO: 62)
DIQMTQSPST LSASVGDRVT ITCRASQSIS SWLAWYQQKP GKAPKLLIYE ASSLESGVPS
RFSGSGSGTE FTLTISSLQP DDFATYYCQQ YNSFPPTFGG GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC

Such PD-1 inhibitors are also described in U.S. Pat. No. 8,354,509 B2 and U.S. Pat. No. 8,779,105 B2, and the PD-1 inhibitors (in particular anti-PD-1 antibodies) of U.S. Pat. No. 8,354,509 B2 and U.S. Pat. No. 8,779,105 B2 are incorporated herein by reference.

In one embodiment, wherein the PD-1 inhibitor is an antibody or antigen binding fragment thereof, the antibodies or antigen-binding fragments that specifically bind to PD-1 or PD-L1 and are comprised in the combination therapy of the invention comprise an antibody Fc-region. It will be appreciated by a skilled person that the Fc portion may be from an IgG antibody, or from a different class of antibody (such as IgM, IgA, IgD or IgE). In one embodiment, the Fc region is from an IgG1, IgG2, IgG3 or IgG4 antibody. Advantageously, however, the Fc region is from an IgG4 antibody.

The Fc region may be naturally-occurring (e.g. part of an endogenously produced antibody) or may be artificial (e.g. comprising one or more point mutations relative to a naturally-occurring Fc region). A variant of an Fc region typically binds to Fc receptors, such as FcγR and/or neonatal Fc receptor (FcRn) with altered affinity providing for improved function and/or half-life of the polypeptide. The biological function and/or the half-life may be either increased or a decreased relative to the half-life of a polypeptide comprising a native Fc region. Examples of such biological functions which may be modulated by the presence of a variant Fc region include antibody dependent cell cytotoxicity (ADCC), antibody-dependent cellular phagocytosis (ADCP), complement-dependent cytotoxicity (CDC), and/or apoptosis.

Thus, the Fc region may be naturally-occurring (e.g. part of an endogenously produced human antibody) or may be artificial (e.g. comprising one or more point mutations relative to a naturally-occurring human Fc region).

As is well documented in the art, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP).

Fc regions may be engineered as described above in relation to the CD137 antibodies of the combination therapy of the invention.

### Antibodies and Inhibitors

The following definitions apply to either or both of the CD137 and PD-1 inhibitors of the invention, wherein the PD-1 inhibitor is an antibody (either an antibody specific for PD-1 or an antibody specific for PD-L1).

The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (C1q) of the classical complement system.

By “an antibody or an antigen-binding fragment thereof” we include substantially intact antibody molecules, as well as chimeric antibodies, humanised antibodies, isolated human antibodies, single chain antibodies, bispecific antibodies, antibody heavy chains, antibody light chains, homodimers and heterodimers of antibody heavy and/or light chains, and antigen-binding fragments and derivatives of the same. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]). The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

For example, the antigen-binding fragment may comprise an scFv molecule, i.e. wherein the VH and VL partner domains are linked via a flexible oligopeptide.

Heavy chains can be of any isotype, including IgG (IgG1, IgG2, IgG3 and IgG4 subtypes), IgA (IgA1 and IgA2 subtypes), IgM and IgE.

Light chains include kappa chains and lambda chains.

Antibodies include, but are not limited to, synthetic antibodies, monoclonal antibodies, single domain antibodies, single chain antibodies, recombinantly produced antibodies, multi-specific antibodies (including bi-specific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

Of particular relevance are antibodies and their antigen-binding fragments that have been “isolated” so as to exist in a physical milieu distinct from that in which it may occur in nature or that have been modified so as to differ from a naturally occurring antibody in amino acid sequence

The phrase “an antibody or an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009, Curr Opin Chem Biol 13(3): 245-255 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Persons skilled in the art will further appreciate that the invention also encompasses combination therapies comprising modified versions of antibodies and antigen-binding fragments thereof, whether existing now or in the future, e.g. modified by the covalent attachment of polyethylene glycol or another suitable polymer (see below).

An antibody may be a polyclonal antibody or a monoclonal antibody. The antibody may be produced by any suitable method.

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299, the disclosures of which are incorporated herein by reference) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120, the disclosures of which are incorporated herein by reference).

Suitable methods for the production of monoclonal antibodies are also disclosed in “Monoclonal Antibodies: A manual of techniques”, H Zola (CRC Press, 1988, the disclosures of which are incorporated herein by reference) and in “Monoclonal Hybridoma Antibodies: Techniques and Applications”, J G R Hurrell (CRC Press, 1982, the disclosures of which are incorporated herein by reference).

Likewise, antibody fragments can be obtained using methods well known in the art (see, for example, Harlow & Lane, 1988, “Antibodies: A Laboratory Manual”, Cold Spring Harbor Laboratory, New York, the disclosures of which are incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

The term “antigen-binding portion” or “antigen-binding fragment” of an antibody refers to one or more fragments of an antibody that retain the ability to specifically bind to an antigen, such as CD137, PD-1 or PD-L1. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

The terms “binding activity” and “binding affinity” are intended to refer to the tendency of an molecule (e.g. an antibody molecule) to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule.

Typically, the Kd for the antibody with respect to the target will be 2-fold, preferably 5-fold, more preferably 10-fold less than Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

An anti-CD137 antibody used in the combination therapies and methods of the invention is preferably capable of binding to its target with an affinity that is at least two-fold, 10-fold, 50-fold, 100-fold or greater than its affinity for binding to another non-target molecule.

A PD-1 inhibitor (such as anti-PD-1 antibody or anti-PD-L1 antibody) used in the combination therapies and methods of the invention is preferably capable of binding to its target with an affinity that is at least two-fold, 10-fold, 50-fold, 100-fold or greater than its affinity for binding to another non-target molecule.

An antibody for use in the methods of the invention may be a human antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences—such antibodies are typically referred to as chimeric or humanised.

A human antibody for use the methods of the invention is typically a human monoclonal antibody. Such a human monoclonal antibody may be produced by a hybridoma which includes a B cell obtained from a transgenic nonhuman animal, e.g., a transgenic mouse, having a genome comprising a human heavy chain transgene and a light chain transgene fused to an immortalized cell. Human antibodies may also be prepared by in vitro immunisation of human lymphocytes followed by transformation of the lymphocytes with Epstein-Barr virus. The term “human antibody derivatives” refers to any modified form of the human antibody, e.g., a conjugate of the antibody and another agent or antibody.

An antibody for use in the methods of the invention may alternatively be a humanised antibody.

The term “humanised” refers to an antibody molecule, generally prepared using recombinant techniques, having an antigen binding site derived from an immunoglobulin from a non-human species and a remaining immunoglobulin structure based upon the structure and/or sequence of a human immunoglobulin. The antigen-binding site may comprise either complete non-human antibody variable domains fused to human constant domains, or only the complementarity determining regions (CDRs) of such variable domains grafted to appropriate human framework regions of human variable domains. The framework residues of such humanised molecules may be wild type (e.g., fully human) or they may be modified to contain one or more amino acid substitutions not found in the human antibody whose sequence has served as the basis for humanization. Humanization lessens or eliminates the likelihood that a constant region of the molecule will act as an immunogen in human individuals, but the possibility of an immune response to the foreign variable region remains (LoBuglio, A. F. et al. (1989) “Mouse/Human Chimeric Monoclonal Antibody In Man: Kinetics And Immune Response,” Proc. Natl. Acad. Sci. (U.S.A.) 86:4220-4224). Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the antigens in question and determine binding capability, flanked by four framework regions (FRs) which are relatively conserved in a given species and which putatively provide a scaffolding for the CDRs. When nonhuman antibodies are prepared with respect to a particular antigen, the variable regions can be “reshaped” or “humanised” by grafting CDRs derived from nonhuman antibody on the FRs present in the human antibody to be modified. Application of this approach to various antibodies has been reported by Sato, K. et al. (1993) Cancer Res 53:851-856. Riechmann, L. et al. (1988) “Reshaping Human Antibodies for Therapy,” Nature 332:323-327; Verhoeyen, M. et al. (1988) “Reshaping Human Antibodies: Grafting An Antilysozyme Activity,” Science 239:1534-1536; Kettleborough, C. A. et al. (1991) “Humanization Of A Mouse Monoclonal Antibody By CDR-Grafting: The Importance Of Framework Residues On Loop Conformation,” Protein Engineering 4:773-3783; Maeda, H. et al. (1991) “Construction Of Reshaped Human Antibodies With HIV-Neutralizing Activity,” Human Antibodies Hybridoma 2:124-134; Gorman, S. D. et al. (1991) “Reshaping A Therapeutic CD4 Antibody,” Proc. Natl. Acad. Sci. (U.S.A.) 88:4181-4185; Tempest, P. R. et al. (1991) “Reshaping A Human Monoclonal Antibody To Inhibit Human Respiratory Syncytial Virus Infection in vivo,” Bio/Technology 9:266-271; Co, M. S. et al. (1991) “Humanized Antibodies For Antiviral Therapy,” Proc. Nati. Acad. Sci. (U.S.A.) 88:2869-2873; Carter, P. et al. (1992) “Humanization Of An Anti-p185her2 Antibody For Human Cancer Therapy,” Proc. Natl. Acad. Sci. (U.S.A.) 89:4285-4289; and Co, M. S. et al. (1992) “Chimeric And Humanized Antibodies With Specificity For The CD33 Antigen,” J. Immunol. 148:1149-1154. In some embodiments, humanised antibodies preserve all CDR sequences (for example, a humanized mouse antibody which contains all six CDRs from the mouse antibodies). In other embodiments, humanised antibodies have one or more CDRs (one, two, three, four, five, six) which are altered with respect to the original antibody, which are also termed one or more CDRs “derived from” one or more CDRs from the original antibody. The ability to humanise an antigen is well known (see, e.g., U.S. Pat. Nos. 5,225,539; 5,530,101; 5,585,089; 5,859,205; 6,407,213; 6,881,557).

The antibody may be or may comprise a variant or a fragment of one of the specific antibodies disclosed herein, provided that said variant or fragment retains specificity for its target. For example, the antibody may be or may comprise a variant or a fragment of one of the specific anti-CD137 antibodies disclosed herein, provided that said variant or fragment retains specificity for CD137. Alternatively or additionally, the antibody may be or may comprise a variant or a fragment of one of the specific anti-PD-1 or PD-L1 antibodies disclosed herein, provided that said variant or fragment retains specificity for PD-1 or PD-1.

A fragment is preferably an antigen binding portion of a said antibody. A fragment may be made by truncation, e.g. by removal of one or more amino acids from the N and/or C-terminal ends of a polypeptide. Up to 10, up to 20, up to 30, up to 40 or more amino acids may be removed from the N and/or C terminal in this way.

Fragments may also be generated by one or more internal deletions.

A variant may comprise one or more substitutions, deletions or additions with respect to the sequences of a specific anti-CD137 antibody or other antibody (e.g. anti-PD-1 antibody or anti-PD-L1 antibody) disclosed herein. A variant may comprise 1, 2, 3, 4, 5, up to 10, up to 20, up to 30 or more amino acid substitutions and/or deletions from the specific sequences disclosed herein. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid.

Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

Preferred “variants” include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Variants may be prepared during synthesis of the antibody or by post-production modification, or when the antibody is in recombinant form using the known techniques of site-directed mutagenesis, random mutagenesis, or enzymatic cleavage and/or ligation of nucleic acids.

Preferably variant antibodies have an amino acid sequence which has more than 60%, or more than 70%, e.g. 75 or 80%, preferably more than 85%, e.g. more than 90 or 95% amino acid identity to the VL or VH domain of an antibody disclosed herein. This level of amino acid identity may be seen across the full length of the relevant SEQ ID NO sequence or over a part of the sequence, such as across 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:
Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

An anti-CD137 antibody or PD-1 inhibitor for use in the combination therapies and methods of the invention may bind to the same epitope as a specific antibody as disclosed herein (e.g. an anti-CD137 antibody may bind domain 2 of CD137), since such an antibody is likely to mimic the action of the disclosed antibody. Whether or not an antibody binds to the same epitope as another antibody may be determined by routine methods. For example, the binding of each antibody to a target may be using a competitive binding assay. Methods for carrying out competitive binding assays are well known in the art. For example they may involve contacting together an antibody and a target molecule under conditions under which the antibody can bind to the target molecule. The antibody/target complex may then be contacted with a second (test) antibody and the extent to which the test antibody is able to displace the first antibody from antibody/target complexes may be assessed. Such assessment may use any suitable technique, including, for example, Surface Plasmon Resonance, ELISA, or flow cytometry. The ability of a test antibody to inhibit the binding of a first antibody to the target demonstrates that the test antibody can compete with said first antibody for binding to the target and thus that the test antibody binds to the same epitope or region on the target as the first antibody, and may therefore mimic the action of the first antibody.

Any antibody referred to herein may be provided in isolated form or may optionally be provided linked (directly or indirectly) to another moiety. The other moiety may be a therapeutic molecule such as a cytotoxic moiety or a drug.

The therapeutic molecule may be directly attached, for example by chemical conjugation, to an antibody of the invention. Methods for conjugating molecules to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety.

Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

The antibody or antigen-binding fragment is linked to a cytotoxic moiety which is a directly cytotoxic chemotherapeutic agent. Optionally, the cytotoxic moiety is a directly cytotoxic polypeptide. Cytotoxic chemotherapeutic agents are well known in the art.

Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfane; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

The cytotoxic moiety may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the antibody, antigen-binding fragment, variant, fusion or derivative thereof in known ways. For example, EDTA or another chelating agent may be attached to the binding moiety and used to attach 111In or 90Y. Tyrosine residues may be directly labelled with 125I or 131I.

The cytotoxic moiety may be a suitable indirectly-cytotoxic polypeptide. The indirectly cytotoxic polypeptide may be a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. The cytotoxic moiety may be capable of converting a non-cytotoxic prodrug into a cytotoxic drug.

The enzyme and prodrug of the system using a targeted enzyme as described herein may be any of those previously proposed. The cytotoxic substance may be any existing anti-cancer drug such as an alkylating agent; an agent which intercalates in DNA; an agent which inhibits any key enzymes such as dihydrofolate reductase, thymidine synthetase, ribonucleotide reductase, nucleoside kinases or topoisomerase; or an agent which effects cell death by interacting with any other cellular constituent. Etoposide is an example of a topoisomerase inhibitor.

Reported prodrug systems include those listed in Table 2.

TABLE 2

| Enzyme | Prodrug |
|---|---|
| Carboxypeptidase G2 | Derivatives of L-glutamic acid and benzoic acid |
| | mustards, aniline mustards, phenol mustards and |
| | phenylenediamine mustards; fluorinated |
| | derivatives of these |
| Alkaline phosphatase | Etoposide phosphate |
| | Mitomycin phosphate |
| Beta-glucuronidase | p-Hydroxyaniline mustard-glucuronide |
| | Epirubicin-glucuronide |
| Penicillin-V-amidase | Adriamycin-N phenoxyacetyl |
| Penicillin-G-amidase | N-(4′-hydroxyphenyl acetyl) palytoxin |
| | Doxorubicin and melphalan |
| Beta-lactamase | Nitrogen mustard-cephalosporin |
| | p-phenylenediamine; doxorubicin derivatives; |
| | vinblastine derivative-cephalosporin, |
| | cephalosporin mustard; a taxol derivative |
| Beta-glucosidase | Cyanophenylmethyl-beta-D-gluco- |
| | pyranosiduronic acid |
| Nitroreductase | 5-(Azaridin-1-yl-)-2,4-dinitrobenzamide |
| Cytosine deaminase | 5-Fluorocytosine |
| Carboxypeptidase A | Methotrexate-alanine |

Suitable enzymes for forming part of an enzymatic portion include: exopeptidases, such as carboxypeptidases G, G1 and G2 (for glutamylated mustard prodrugs), carboxypeptidases A and B (for MTX-based prodrugs) and aminopeptidases (for 2-α-aminocyl MTC prodrugs); endopeptidases, such as e.g. thrombolysin (for thrombin prodrugs); hydrolases, such as phosphatases (e.g. alkaline phosphatase) or sulphatases (e.g. aryl sulphatases) (for phosphylated or sulphated prodrugs); amidases, such as penicillin amidases and arylacyl amidase; lactamases, such as β-lactamases; glycosidases, such as β-glucuronidase (for β-glucuronomide anthracyclines), α-galactosidase (for amygdalin) and β-galactosidase (for β-galactose anthracycline); deaminases, such as cytosine deaminase (for 5FC); kinases, such as urokinase and thymidine kinase (for gancyclovir); reductases, such as nitroreductase (for CB1954 and analogues), azoreductase (for azobenzene mustards) and DT-diaphorase (for CB1954); oxidases, such as glucose oxidase (for glucose), xanthine oxidase (for xanthine) and lactoperoxidase; DL-racemases, catalytic antibodies and cyclodextrins.

Preferably, the prodrug is relatively non-toxic compared to the cytotoxic drug. Typically, it has less than 10% of the toxicity, preferably less than 1% of the toxicity as measured in a suitable in vitro cytotoxicity test.

It is likely that the moiety which is able to convert a prodrug to a cytotoxic drug will be active in isolation from the rest of the agent of the invention but it is necessary only for it to be active when (a) it is in combination with the rest of the agent of the invention and (b) the agent of the invention is attached to, adjacent to or internalised in target cells.

When each moiety is a polypeptide, the two portions may be linked together by any of the conventional ways of cross-linking polypeptides. For example, the antibody or antigen-binding fragment may be enriched with thiol groups and the further moiety reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

The cytotoxic moiety may be a radiosensitizer. Radiosensitizers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole. Also, delivery of genes into cells can radiosensitise them, for example delivery of the p53 gene or cyclin D. The further moiety may be one which becomes cytotoxic, or releases a cytotoxic moiety, upon irradiation. For example, the boron-10 isotope, when appropriately irradiated, releases a particles which are cytotoxic. Similarly, the cytotoxic moiety may be one which is useful in photodynamic therapy such as photofrin.

### Methods and Combination Therapies

The invention provides a combination therapy for use in treating cancer, such as a solid tumour, in a subject comprising (a) an antibody, or antigen-binding portion thereof, that specifically binds to CD137, and (b) a further immunotherapeutic agent, wherein the further immunotherapeutic agent is a PD-1 inhibitor.

The antibody, or antigen-binding portion thereof, that specifically binds to CD137 and the PD-1 inhibitor are as described above.

It will be appreciated by persons skilled in the art that the presence of the two active agents (as detailed above) may provide a synergistic benefit in the treatment of a solid tumour in a subject. By “synergistic” we include that the therapeutic effect of the two agents in combination (e.g. as determined by reference to the rate of growth or the size of the tumour) is greater than the additive therapeutic effect of the two agents administered on their own. Such synergism can be identified by testing the active agents, alone and in combination, in a relevant cell line model of the solid tumour.

The terms “combination therapy” or “combined treatment” or “in combination” as used herein denotes any form of simultaneous or sequential treatment with at least two different therapeutic agents.

According to certain embodiments, the anti-CD137 antibody, or antigen-binding fragment thereof, and the PD-1 inhibitor are administered simultaneously, either in the same composition or in separate compositions. According to other embodiments, the anti-CD137 antibody, or antigen-binding fragment thereof, and the PD-1 inhibitor are administered sequentially, i.e., the anti-CD137 antibody, or antigen-binding fragment thereof, is administered either prior to or after the administration of the PD-1 inhibitor. In some embodiments, the administration of the anti-CD137 antibody, or antigen-binding fragment thereof, and the PD-1 inhibitor are concurrent, i.e., the administration period of the anti-CD137 antibody, or antigen-binding fragment thereof, and that of the PD-1 inhibitor overlap with each other. In some embodiments, the administration of the anti-CD137 antibody, or antigen-binding fragment thereof, and the PD-1 inhibitor are non-concurrent, or sequential. For example, in some embodiments, the administration of the anti-CD137 antibody, or antigen-binding fragment thereof, is terminated before the PD-1 inhibitor is administered. In some embodiments, the administration of the PD-1 inhibitor is terminated before the anti-CD137 antibody, or antigen-binding fragment thereof, is administered.

According to certain typical embodiments, the anti-CD137 antibody, or antigen-binding fragment thereof, and the PD-1 inhibitor are administered as a single therapeutic composition. According to some embodiments, the therapeutic composition further comprises therapeutically acceptable diluents or carrier.

The combination of a systemic PD-1 inhibitor and anti-CD137 antibody is not just attractive because of the expression of PD-L1 by tumours. CD137 stimulation results in activation of tumour infiltrating T cells enabling them to kill the tumour cells. However, CD137 mediated activation of T cells also results in an upregulation of PD-1 on T cells (and indirectly to upregulation of PD-L1 on tumour cells). Conversely, PD-1 inhibition results in re-activation (or removal of inhibition of) tumour specific T cells. T cells that are re-activated by a PD-1 inhibitor express CD137. CD137 stimulation of CD137 expressing T cells increase their ability to kill tumor cells and prevent them from being exhausted (or rescue them from exhaustion)

Thus, the further immunotherapeutic agent is a PD-1 inhibitor and may preferably be an antibody or other agent which specifically binds to at least one of PD-1 or PD-L1 (as already described above).

Where the further immunotherapeutic agent is an antibody or bi-specific molecule comprising an antibody, it will be understood that all of the general considerations set out above regarding the definitions of antibodies, antigen-binding fragments of antibodies, optional conjugation to additional therapeutic moieties etc, also apply to an antibody that is the further immunotherapeutic agent. Similarly, it will be understood that the definitions of target specificity/affinity and methods for determining specificity/affinity set out above for anti-CD137 antibodies will apply equally to an antibody that is the further immunotherapeutic agent, except the specific target of the agent will be read in place of CD137. Variants and fragments of an antibody which is the further immunotherapeutic agent may also be defined in the same way as the variants and fragments of anti-CD137 antibodies.

The invention provides a method for treating a cancer, preferably a solid tumour, in a subject. The tumour is typically malignant and may be metastatic.

In one embodiment, the combination therapy of the invention may be used to treat patients or subjects who suffer from or are at risk of suffering from a cancer.

By ‘treatment’ we include both therapeutic and prophylactic treatment of the patient. The term ‘prophylactic’ is used to encompass the use of an agent, or formulation thereof, as described herein which either prevents or reduces the likelihood of a cancer, or the spread, dissemination, or metastasis of cancer cells in a patient or subject. The term ‘prophylactic’ also encompasses the use of an agent, or formulation thereof, as described herein to prevent recurrence of a cancer in a patient who has previously been treated for the neoplastic disorder.

The cancer may be associated with formation of solid tumours or may be a haematologic cancer. Cancer types that may be treated include carcinomas, sarcomas, lymphomas, leukemias, blastomas and germ cell tumours.

The cancer may be selected from the group consisting of prostate cancer; breast cancer; colorectal cancer; kidney cancer; pancreatic cancer; ovarian cancer; lung cancer; cervical cancer; rhabdomyosarcoma; neuroblastoma; bone cancer; multiple myeloma; leukemia (such as acute lymphoblastic leukemia [ALL] and acute myeloid leukemia [AML]), skin cancer (e.g. melanoma), bladder cancer and glioblastoma.

In one embodiment, the cancer may be selected from the list of cancers in Table 4 or Table 5 below (taken from WO 2018/091740).

TABLE 4
Mean expression values of solid human tumors with an above
average expression (mean expression level ≥10) of both
Fcγ receptor and CD137 (TNFRSF9). The ten tumors with
the highest expression of the six Fcγ receptors are shown.

| | FcγRIA | TNFRSF9 |
|---|---|---|
| | (Mean | (Mean |
| Cancers with cells expressing FcγRIA | expression | expression |
| and TNFRSF9 | level) | level) |
| fibrous histiocytoma, malignant, | 13.43 | 10.22 |
| metastatic | | |
| adenosquamous carcinoma | 12.78 | 10.80 |
| undifferentiated sarcoma | 12.67 | 10.16 |
| clear cell adenocarcinoma, NOS, | 12.66 | 10.62 |
| metastatic | | |
| acinar cell carcinoma | 12.47 | 10.82 |
| dedifferentiated liposarcoma | 12.45 | 10.67 |
| renal cell carcinoma, unstated behavior | 12.44 | 10.38 |
| intraductal micropapillary carcinoma | 12.36 | 10.68 |
| fibrous histiocytoma, malignant | 12.35 | 11.05 |
| large cell neuroendocrine carcinoma | 12.35 | 10.29 |
| | FcγRIIA | TNFRSF9 |
| | (Mean | (Mean |
| Cancers with cells expressing | expression | expression |
| FcγRIIA and TNFRSF9 | level) | level) |
| Langerhans-cell histiocytosis, unifocal | 14.58 | 13.85 |
| acinar cell carcinoma | 13.55 | 10.82 |
| fibrous histiocytoma, malignant, | 13.54 | 10.22 |
| metastatic | | |
| undifferentiated sarcoma | 13.48 | 10.16 |
| adenocarcinoma with mixed subtypes | 13.43 | 10.77 |
| dedifferentiated liposarcoma | 13.32 | 10.67 |
| undifferentiated sarcoma | 13.22 | 10.52 |
| fibrous histiocytoma, malignant | 13.10 | 11.05 |
| adenosquamous carcinoma | 13.01 | 10.80 |
| dedifferentiated liposarcoma | 12.95 | 10.36 |
| | FcγRIIB | TNFRSF9 |
| | (Mean | (Mean |
| Cancers with cells expressing | expression | expression |
| FcγRIIB and TNFRSF9 | level) | level) |
| Langerhans-cell histiocytosis, unifocal | 13.23 | 13.85 |
| fibrous histiocytoma, malignant, | 12.64 | 10.22 |
| metastatic | | |
| adenocarcinoma with mixed subtypes | 12.42 | 10.77 |
| adenosquamous carcinoma | 12.22 | 10.80 |
| acinar cell carcinoma | 12.19 | 10.82 |
| Hodgkin's disease, NOS | 12.01 | 10.88 |
| renal cell carcinoma, unstated behavior | 12.00 | 10.38 |
| dedifferentiated liposarcoma | 11.98 | 10.67 |
| papillary adenocarcinoma, NOS | 11.81 | 10.02 |
| undifferentiated sarcoma | 11.76 | 10.16 |
| | FcγRIIC | TNFRSF9 |
| | (Mean | (Mean |
| Cancers with cells expressing | expression | expression |
| FcγRIIC and TNFRSF9 | level) | level) |
| Langerhans-cell histiocytosis, unifocal | 12.72 | 13.85 |
| | FcγRIIIA | TNFRSF9 |
| | (Mean | (Mean |
| Cancers with cells expressing | expression | expression |
| FcγRIIIA and TNFRSF9 | level) | level) |
| fibrous histiocytoma, malignant, | 14.14 | 10.22 |
| metastatic | | |
| acinar cell carcinoma | 13.14 | 10.82 |
| large cell neuroendocrine carcinoma | 12.97 | 10.29 |
| adenocarcinoma with mixed subtypes | 12.91 | 10.77 |
| renal cell carcinoma, unstated behavior | 12.85 | 10.38 |
| intraductal micropapillary carcinoma | 12.83 | 10.68 |
| carcinoma, NOS | 12.70 | 10.19 |
| carcinoma, NOS | 12.68 | 10.06 |
| undifferentiated sarcoma | 12.52 | 10.16 |
| carcinoma, NOS | 12.42 | 10.10 |
| | FcγRIIIB | TNFRSF9 |
| | (Mean | (Mean |
| Cancers with cells expressing | expression | expression |
| FcγRIIIB and TNFRSF9 | level) | level) |
| carcinoma, NOS | 13.09 | 10.19 |
| carcinoma, NOS | 12.66 | 10.06 |
| carcinoma, NOS | 12.61 | 10.10 |
| tubular adenocarcinoma | 12.06 | 10.18 |
| carcinoma, NOS, micro-dissected | 12.00 | 10.92 |
| adenocarcinoma, intestinal type | 11.97 | 10.26 |
| fibrous histiocytoma, malignant, | 11.89 | 10.22 |
| metastatic | | |
| neoplasm, malignant | 11.81 | 10.05 |
| adenocarcinoma, NOS | 11.74 | 10.06 |
| renal cell carcinoma, unstated behavior | 11.61 | 10.38 |

TABLE 5
Mean expression values of hematological malignancies with an
above average expression (mean expression level ≥10) of
both Fcγ receptor and CD137. The ten malignancies with
the highest expression of the six Fcγ receptors are shown.

| | FcγRIA | TNFRSF9 |
|---|---|---|
| | (Mean | (Mean |
| Cancers with cells expressing FcγRIA and | expression | expression |
| TNFRSF9 | level) | level) |
| (extranodal) NK/T-cell lymphoma, nasal and nasal-type | 13.97 | 11.56 |
| Hodgkin's disease, NOS | 13.65 | 10.88 |
| malignant lymphoma, large B-cell, diffuse | 12.15 | 12.23 |
| primary mediastinal B-cell lymphoma | 12.13 | 12.43 |
| anaplastic large cell lymphoma, T-cell and Null cell type | 12.11 | 11.38 |
| (ALCL), unstated behavior | | |
| angioimmunoblastic T-cell lymphoma | 11.93 | 13.85 |
| mature T-cell lymphoma, NOS | 11.86 | 12.53 |
| mature T-cell lymphoma, NOS, unstated behavior | 11.69 | 11.72 |
| anaplastic large cell lymphoma, T-cell and Null cell type | 11.67 | 11.43 |
| (ALCL) | | |
| angioimmunoblastic T-cell lymphoma, unstated | 11.63 | 12.98 |
| behavior | | |
| | FcγRIIA | TNFRSF9 |
| | (Mean | (Mean |
| Cancers with cells expressing FcγRIIA and | expression | expression |
| TNFRSF9 | level) | level) |
| mycosis fungoides | 13.27 | 11.34 |
| Hodgkin's disease, NOS | 12.33 | 10.88 |
| (extranodal) NK/T-cell lymphoma, nasal and nasal-type | 12.26 | 11.56 |
| mature T-cell lymphoma, NOS | 11.76 | 12.53 |
| angioimmunoblastic T-cell lymphoma | 11.69 | 13.85 |
| malignant lymphoma, large B-cell, diffuse | 11.37 | 12.23 |
| angioimmunoblastic T-cell lymphoma, unstated | 11.34 | 12.98 |
| behavior | | |
| mature T-cell lymphoma, NOS, unstated behavior | 11.33 | 11.72 |
| adult T-cell leukemia/lymphoma (HTLV-1 positive), | 11.28 | 11.13 |
| unstated behavior | | |
| anaplastic large cell lymphoma, T-cell and Null cell type | 11.23 | 11.38 |
| (ALCL), unstated behavior | | |
| | FcγRIIB | TNFRSF9 |
| | (Mean | (Mean |
| Cancers with cells expressing FcγRIIB and | expression | expression |
| TNFRSF9 | level) | level) |
| chronic lymphocytic B-cell leukemia, unstated behavior, | 14.69 | 10.46 |
| micro-dissected | | |
| mantle cell lymphoma | 14.06 | 10.11 |
| (extranodal) marginal zone B-cell lymphoma, NOS | 12.73 | 11.85 |
| malignant lymphoma, nodular, NOS, unstated behavior | 12.69 | 12.97 |
| malignant lymphoma, nodular, NOS | 12.16 | 12.02 |
| Hodgkin's disease, NOS | 12.01 | 10.88 |
| malignant lymphoma, large B-cell, diffuse | 11.97 | 12.23 |
| angioimmunoblastic T-cell lymphoma | 11.89 | 13.85 |
| mycosis fungoides | 11.76 | 11.34 |
| Hodgkin's disease, NOS, micro-dissected | 11.38 | 11.32 |

| | FcγRIIC | TNFRSF9 |
|---|---|---|
| | (Mean | (Mean |
| Cancers with cells expressing FcγRIIC and | expression | expression |
| TNFRSF9 | level) | level) |
| juvenile myelomonocytic leukemia | 10.00 | 10.00 |

| | FcγRIIIA | TNFRSF9 |
|---|---|---|
| | (Mean | (Mean |
| Cancers with cells expressing FcγRIIIA and | expression | expression |
| TNFRSF9 | level) | level) |
| juvenile myelomonocytic leukemia | 13.19 | 10.00 |
| (extranodal) NK/T-cell lymphoma, nasal and nasal-type | 12.09 | 11.56 |
| mycosis fungoides | 11.71 | 11.34 |
| mature T-cell lymphoma, NOS | 11.16 | 12.53 |
| anaplastic large cell lymphoma, T-cell and Null cell type | 10.80 | 11.43 |
| (ALCL) | | |
| angioimmunoblastic T-cell lymphoma | 10.65 | 13.85 |
| Hodgkin's disease, NOS | 10.51 | 10.88 |
| anaplastic large cell lymphoma, T-cell and Null cell type | 10.38 | 11.38 |
| (ALCL), unstated behavior | | |
| malignant lymphoma, large B-cell, diffuse | 10.31 | 12.23 |
| primary mediastinal B-cell lymphoma | 10.26 | 12.43 |
| | FcγRIIIB | TNFRSF9 |
| | (Mean | (Mean |
| Cancers with cells expressing FcγRIIIB and | expression | expression |
| TNFRSF9 | level) | level) |
| juvenile myelomonocytic leukemia | 15.03 | 10.00 |
| (extranodal) NK/T-cell lymphoma, nasal and nasal-type | 11.95 | 11.56 |
| anaplastic large cell lymphoma, T-cell and Null cell type | 11.85 | 11.38 |
| (ALCL), unstated behavior | | |
| mature T-cell lymphoma, NOS | 11.57 | 12.53 |
| mature T-cell lymphoma, NOS, unstated behavior | 11.56 | 11.72 |
| angioimmunoblastic T-cell lymphoma, unstated | 11.54 | 12.98 |
| behavior | | |
| primary mediastinal B-cell lymphoma | 11.44 | 12.43 |
| angioimmunoblastic T-cell lymphoma | 11.30 | 13.85 |
| adult T-cell leukemia/lymphoma (HTLV-1 positive), | 11.08 | 11.13 |
| unstated behavior | | |
| (extranodal) marginal zone B-cell lymphoma, NOS | 10.95 | 11.85 |

Typically, the therapeutic agents in the combination therapy of the invention may be administered in parenteral form, for example by injection into the bloodstream or at/near the site of a tumour. Typically, the therapeutic agents in the combination therapy of the invention are administered intravenously.

In one embodiment, the combination therapy and or methods of the invention can be used for treating a patient who has been pre-screened and identified as having a tumour with cells expressing CD137 and FcγR, such as FcγRI, FcγRIIA, FcγRIIB or combinations thereof.

It will be further appreciated that the combination therapy of the invention may be used as a sole treatment for cancer in a patient or as part of an additional combination treatment (which additional treatment may be a pharmaceutical agent, radiotherapy and/or surgery).

The cancer may be a solid tumour. Solid tumours are classically defined by the tissue from which they originate, e.g. breast, colon etc. However, since immunotherapy acts on the immune system, and not the tumour itself, the immune status of the tumour may be more predictive of the response than the origin of the tumour. In the supporting studies presented herein, the MC38 colon cancer model is evaluated in more detail, which is generally immunogenic and responds to PD-1 therapy alone.

In one embodiment of the present invention, the tumour is immunogenic. Such tumours are characterised by infiltration of immune cells, such as T cells and cells of myeloid origin. It has been demonstrated that infiltration of CD8 T cells, i.e. a more immunogenic tumour profile, correlates with a good prognosis following therapy, for example in colon cancer, (Galon et al., 2014, J. Pathol. 232(2):199-209).

In an alternative embodiment of the invention, the tumour is non-immunogenic or poorly-immunogenic. Poorly immunogenic tumours often have low or absent MHCI expression and are characterized by lower number of infiltrating immune cells, such as T cells and cells of myeloid origin (Lechner et al., 2013, J Immunotherapy 36(9):477-89). The tumour may be an adenoma, an adenocarcinoma, a blastoma, a carcinoma, a desmoid tumour, a desmopolastic small round cell tumour, an endocrine tumour, a germ cell tumour, a lymphoma, a sarcoma, a Wilms tumour, a lung tumour, a colon tumour, a lymph tumour, a breast tumour or a melanoma.

Types of blastoma include hepatblastoma, glioblastoma, neuroblastoma or retinoblastoma. Types of carcinoma include breast, endometrial, colorectal carcinoma or hepatocellular carcinoma, pancreatic, prostate, gastric, urothelial, renal, Merkel cell, oesophageal, cervical, and head and neck carcinomas, and adenocarcinoma. Types of sarcoma include Ewing sarcoma, osteosarcoma, rhabdomyosarcoma, or any other soft tissue sarcoma. Types of melanoma include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma. Types of lymphoma include Precursor T-cell leukemia/lymphoma, Follicular lymphoma, Diffuse large B cell lymphoma, Mantle cell lymphoma, B-cell chronic lymphocytic leukemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

In an embodiment of the invention, the cancer may be a microsatellite instability high (MSI-H) cancer, and/or a deficient mismatch repair (dMMR) cancer, and/or a cancer associated with a high tumour mutational burden (i.e. TMB-high).

In an embodiment of the invention, the cancer may be a mesothelioma.

The method of the invention comprises (a) administering to the subject a therapeutically effective amount of an antibody that specifically binds to CD137, and (b) administering to the subject a therapeutically effective amount of an further immunotherapeutic agent, wherein the further immunotherapeutic agent is a PD-1 inhibitor, optionally wherein the PD-1 inhibitor is administered systemically. Steps (a) and (b) may be carried out simultaneously. Alternatively steps (a) and (b) may be carried our sequentially provided step (a) precedes step (b). In step (a), the anti-CD137 antibody is preferably administered systemically to the tumour and most preferably the anti-CD137 antibody is administered intravenously.

By “therapeutically effective amount” of a substance, it is meant that a given substance is administered to a subject suffering from a condition, in an amount sufficient to cure, alleviate or partially arrest the condition or one or more of its symptoms. Such therapeutic treatment may result in a decrease in severity of disease symptoms, or an increase in frequency or duration of symptom-free periods. Effective amounts for a given purpose and a given agent will depend on the severity of the disease or injury as well as the weight and general state of the subject. As used herein, the term “subject” includes any mammal, preferably a human.

The invention also provides:
an antibody that specifically binds to CD137 for use in a method for treating a solid tumour in a subject, the method comprising (a) administering to the subject a therapeutically effective amount of said antibody that specifically binds to CD137, and (b) systemically administering to the subject a therapeutically effective amount of a PD-1 inhibitor. Steps (a) and (b) may be carried out simultaneously. Alternatively steps (a) and (b) may be carried our sequentially provided step (a) precedes step (b). In step (a), said anti-CD137 antibody is preferably administered locally to the tumour.
use of an antibody that specifically binds to CD137 in the manufacture of a medicament for treating a solid tumour in a subject, wherein said treating comprises (a) administering to the tumour a therapeutically effective amount of said antibody that specifically binds to CD137, and (b) systemically administering to the subject a therapeutically effective amount of a PD-1 inhibitor. Steps (a) and (b) may be carried out simultaneously. Alternatively steps (a) and (b) may be carried our sequentially provided step (a) precedes step (b). In step (a), said anti-CD137 antibody is preferably administered systemically, and most preferably said anti_CD137 antibody is administered intravenously.
a product containing (1) an antibody that specifically binds to CD137 and (2) a PD-1 inhibitor for simultaneous, separate or sequential use in a method for treating a solid tumour in a subject, the method comprising (a) systemically administering to the tumour a therapeutically effective amount of said antibody that specifically binds to CD137, and optionally (b) systemically administering to the subject a therapeutically effective amount of a PD-1 inhibitor. Steps (a) and (b) may be carried out simultaneously. Alternatively steps (a) and (b) may be carried our sequentially provided step (a) precedes step (b). In step (a), said anti-CD137 antibody is preferably administered locally to the tumour.

### Timing and Order of Steps (a) and (b)

In an embodiment, steps (a) and (b) may be carried out sequentially (i.e. at different times), with step (a) being carried out before step (b).

Steps (a) and (b) may be separated by an interval such that the combined anti-tumour effect is optimised. Step (b) may be conducted a sufficiently long interval after step (a) that at least one physiological effect of step (a) is at or close to its peak level. For example, the anti-CD137 antibody will typically stimulate CD137 and activate T cells and/or other immune cells (e.g. to induce release of interferon gamma from CD8+ cell). The activated T cells may begin to express higher levels of immune system checkpoint molecules (such as PD-1) within around 24 hours of treatment with anti-CD137. These immune system checkpoint molecules may negatively regulate the anti-tumour response. The further immunotherapeutic agent administered in step (b) is a PD-1 inhibitor and may thus preferably be an anti-PD-1 or anti-PDL1 antibody which blocks or inhibits such the activity of PD-1. Where the further immunotherapeutic agent administered in step (b) is such an agent, step (b) may be carried out a sufficiently long interval after step (a) such that the expression level of the immune system checkpoint molecule (such as PD-1) in cells in the subject, or the number of cells in the subject expressing said immune system checkpoint molecule, is elevated relative to said level or number in the subject prior to step (a), or relative to said level or number in in a healthy subject. In this context, step (b) may be conducted within 24 hours after step (a), between 24 hours and two weeks after step (a), between 24 hours and one week after step (a), between 24 hours and 72 hours after step (a), or between 24 hours and 48 hours after step (a). Preferably step (b) is conducted within 24 hours after step (a).

Alternatively step (b) may be conducted at a time point after step (a) where the expression level of the immune system checkpoint molecule (such as PD-1) in a cell of the subject, or the number of cells in the subject expressing said immune system checkpoint molecule, is determined to be elevated relative to said level or number in the subject prior to step (a), or relative to said level or number in in a healthy subject.

The expression level of an immune system checkpoint molecule (such as PD-1) in a cell of a subject, or the number of cells in a subject expressing such a molecule, may be determined by any suitable means, for example by flow cytometric analysis of a sample taken from the subject.

Alternatively, in a most preferred embodiment, steps (a) and (b) are carried out on the same day. It may be preferable to carry out steps (a) and (b) simultaneously (i.e. at the same time), or within 24 hours of each other, such that both steps may be carried out on the same day or during the same visit to a treatment centre. This may be particularly advantageous where access to treatment centres is restricted. In this context, steps (a) and (b) may be carried out simultaneously, or may be carried out less than 24 hours apart, less than 12 hours apart, less than 10 hours apart, less than 6 hours apart, less than 4 hours apart, less than 3 hours apart or less than 2 hours apart.

In a further embodiment, steps (a) and (b) are carried out simultaneously or wherein step (b) is carried out between 24 hours and two weeks after step (a), between 24 hours and one week after step (a), between 24 and 72 hours after step (a), or between 24 and 48 hours after step (a).

In any of the above embodiments, step (a) may be conducted on multiple further instances after the first instance. That is, the subject may receive a series of doses of anti-CD137 antibody. These doses are administered such that the subject has only intermittent exposure to the anti-CD137 antibody, preferably such that the immune cells of the subject do not become depleted and/or that the subject does not suffer from tachyphylaxia to the anti-CD137 antibody. At detection of either of these symptoms, the next administration of anti-CD137 antibody may be delayed or cancelled. If multiple doses of anti-CD137 are administered, step (b) is preferably conducted in a manner which, following initiation of step (b), permits continuous exposure of the subject to the further immunotherapeutic agent (PD-1 inhibitor) for the duration of the method, including during any second and further instances of step (a). This may be particularly appropriate where the additional agent is an anti-PD-1 or anti-PDL1 antibody which blocks or inhibits such the activity of the immune system checkpoint molecule, PD-1. Continuous receptor blockade may be particularly important for the therapeutic effects of such agents.

Thus, in one embodiment, step (a) is conducted on multiple separate occasions and step (b) is conducted such that exposure of the subject to the further immunotherapeutic agent is continuous for the duration of the method.

### Step (a)

Step (a) of the method concerns the local or systemic administration of an anti-CD137 antibody to a subject having a solid tumour. Preferably, step (a) concerns the systemic administration of an anti-CD137 antibody, e.g. via intravenous or subcutaneous administration. In a most preferred embodiment, step (a) involves intravenous administration of the anti-CD137 antibody.

In an alternative embodiment, the anti-CD137 antibody or antigen binding fragment thereof is locally administered to a tumour site in a subject. Local administration to the tumour site includes peritumoral, juxtatumoral, intratumoral, intralesional, perilesional, intracranial and intravesicle administration by any suitable means, such as injection. Local administration may also include intra cavity infusion and inhalation, depending on the site of the tumour.

A high proportion of the anti-CD137 antibody may be retained at the tumour site in vivo, that is within the tumour microenvironment, for an extended period of time following administration of said antibody. That is, the antibody exhibits reduced leakage from the tumour site into vascular or lymphatic circulation, particularly when locally administered to the tumour site. Preferably at least 30% of an antibody dose administered to a tumour in accordance with the method is retained in the tumour site at four hours after administration, more preferably at least 40% of the dose is retained at four hours after administration and most preferably at least 50% of the dose is retained at four hours after administration.

By “retained at the site of a solid tumour” we include that the anti-CD137 antibody is released only slowly from the tumour area. Antibody retention in a tumour microenvironment can be studied by injecting the antibody into tumours in murine models and measuring the serum levels of the antibody over time after administration. Alternatively the distribution of an antibody can be measured using radiolabelled antibodies injected into tumours in murine models. Suitable techniques are known to the skilled person. For example, retention of the antibody at the tumour site may be assessed by monitoring serum levels of the antibody post-administration (see Mangsbo et al., 2014, Clin. Cancer Res. 21(5):1115-1126, the disclosure of which are incorporated herein by reference). For example, in one embodiment, the serum levels of anti-CD137 four hours following intratumoral injection of 30 μg of the antibody (in 60 μL) are less than 1 μL/ml.

### Step (b)

Step (b) of the method concerns the systemic administration of a PD-1 inhibitor to a subject. Systemic administration of any agent described herein (including the anti-CD137 antibody of step (a)) means administration into the circulatory system of the subject, including the vascular and/or lymphatic system. Such administration may be by any suitable route, but is typically parenteral.

Thus, in one embodiment, the PD-1 inhibitor is administered locally to a tumour site in a subject. In one embodiment the PD-1 inhibitor is administered to the subject systemically, e.g. intravenous or sub-cutaneous. In a preferred embodiment, the systemic administration of a PD-1 inhibitor is intravenous.

The phrase “parenteral administration” as used herein means modes of administration other than enteral and topical administration, and is typically achieved by injection, infusion or implantation. Suitable routes include intravenous, intramuscular, intradermal, intraperitoneal, subcutaneous, spinal, intracerebral, intrathecal, intraosseous or other parenteral routes of administration.

### Kits and Pharmaceutical Compositions

The invention also provides a kit for treating a cancer, preferably a solid tumour in a subject, the kit comprising a combination therapy as defined above. For example, the kit may comprise (a) a therapeutically effective amount of an antibody that specifically binds to CD137 and optionally that is retained at the tumour site following administration and (b) a therapeutically effective amount of a PD-1 inhibitor. The antibody that specifically binds to CD137 is preferably provided in a form suitable for local administration to a tumour site.

The kits of the invention may additionally comprise one or more other reagents or instruments which enable any of the embodiments mentioned above to be carried out. Such reagents or instruments include one or more of the following: suitable buffer(s) (aqueous solutions) and means to administer the anti-CD137 antibody and/or the PD-1 inhibitor (such as a vessel or an instrument comprising a needle).

The anti-CD137 antibody and the PD-1 inhibitor used in the methods of the invention, or provided in the kits of the invention, may each be provided as a separate pharmaceutical composition formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible and are also compatible with the required routes of administration.

Thus, the carrier for the anti-CD137 antibody and the PD-1 inhibitor may be suitable for systemic administration, which as defined above means administration into the circulatory system of the subject, including the vascular and/or lymphatic system. Such administration may be by any suitable route, but is typically parenteral. The phrase “parenteral administration” as used herein means modes of administration other than enteral and topical administration, and is typically achieved by injection, infusion or implantation. Suitable routes include intravenous, intramuscular, intradermal, intraperitoneal, subcutaneous, spinal or other parenteral routes of administration.

However, the carrier for the anti-CD137 antibody is preferably suitable for local administration, which as defined above includes peritumoral, juxtatumoral, intratumoral, intralesional, perilesional, intracranial and intravesicle administration by any suitable means, such as injection. Local administration may also include intra cavity infusion and inhalation, depending on the site of the tumour.

Depending on the route of administration, the antibody and/or the agent may be coated in a material to protect the antibody from the action of acids and other natural conditions that may inactivate or denature the antibody and/or agent. Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

It will be appreciated by persons skilled in the art that the antibody components of the combination therapies of the present invention are typically provided in the form of one or more pharmaceutical compositions, each containing a therapeutically-effective amount of the antibody component(s) together with a pharmaceutically-acceptable buffer, excipient, diluent or carrier.

It will be appreciated by persons skilled in the art that additional compounds may also be included in the pharmaceutical compositions, including, chelating agents such as EDTA, citrate, EGTA or glutathione.

By “pharmaceutically acceptable” we mean a non-toxic material that does not decrease the effectiveness of the CD137-binding activity of the antibody polypeptide of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

A ‘therapeutically effective amount’, or ‘effective amount’, or ‘therapeutically effective’, as used herein, refers to that amount which provides a therapeutic effect for a given condition and administration regimen. This is a predetermined quantity of active antibody calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent.

A therapeutically effective amount can be determined by the ordinary skilled medical or veterinary worker based on patient characteristics, such as age, weight, sex, condition, complications, other diseases, etc., as is well known in the art.

A pharmaceutical composition may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin.

Pharmaceutical compositions typically must be sterile and stable under the conditions of manufacture and storage. The composition can be formulated as a solution, microemulsion, liposome, or other ordered structure suitable to high drug concentration. Sterile injectable solutions can be prepared by incorporating the active agent (e.g. antibody) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof. Pharmaceutical compositions may comprise additional active ingredients as well as those mentioned above.

Suitable pharmaceutically acceptable buffers, diluents, carriers and excipients are well-known in the art (see Remington's Pharmaceutical Sciences, 18^{th} edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to include an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to include an aqueous or non-aqueous solution with the purpose of diluting the agent in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to include any compound added to the formulation to increase the biological effect of the agent of the invention. The adjuvant may be one or more of zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The active antibody-based agents of the invention may be formulated into any type of pharmaceutical composition known in the art to be suitable for the delivery thereof.

In one embodiment, the pharmaceutical compositions of the invention may be in the form of a liposome, in which the agent is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871 and in EP 0 213 303, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(carprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of nanoparticles, for example based on poly-gamma glutamic acid. Details of the preparation and use of such nanoparticles can be found in WO 2011/128642, the disclosures of which are incorporated herein by reference. It will be appreciated by persons skilled in the art that one or more of the active components of the combination therapies of the present invention may be formulated in separate nanoparticles, or both active components may be formulated in the same nanoparticles.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polyvinyl imidazole, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the agents may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active agent. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonar, buccal, oral, parenteral, vaginal and rectal. Also administration from implants is possible.

Advantageously, the pharmaceutical composition is suitable for administration at or near the site of a tumour, e.g. intra-tumourally or peri-tumourally.

It is preferred that the pharmaceutical composition is suitable for parenteral administration, for example the pharmaceutical composition is preferably suitable for administration intravenously, intracerebroventricularly, intraarticularly, intra-arterially, intraperitoneally, intrathecally, intraventricularly, intrasternally, intracranially, intramuscularly or subcutaneously, or by infusion techniques. Methods for formulating an antibody into a pharmaceutical composition, such as a pharmaceutical composition suitable for parenteral administration, will be well-known to those skilled in the arts of medicine and pharmacy. Preferred compositions are described in the accompanying Examples.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

The combination therapy of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period. Preferably, delivery is performed intra-muscularly (i.m.) and/or sub-cutaneously (s.c.) and/or intravenously (i.v.).

The combination therapy of the invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for the administration of the combination therapy of the invention. A device which delivers a pulsed electric field to cells increases the permeability of the cell membranes to the drug, resulting in a significant enhancement of intracellular drug delivery.

The combination therapy of the invention can also be delivered by electro-incorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative combination therapy of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The combination therapy of the invention can also be delivered orally. The process employs a natural process for oral uptake of vitamin B₁₂ and/or vitamin D in the body to co-deliver proteins and peptides. By riding the vitamin B₁₂ and/or vitamin D uptake system, the agents, medicaments and pharmaceutical compositions of the invention can move through the intestinal wall. Complexes are synthesised between vitamin B₁₂ analogues and/or vitamin D analogues and the drug that retain both significant affinity for intrinsic factor (IF) in the vitamin B₁₂ portion/vitamin D portion of the complex and significant bioactivity of the active substance of the complex.

The combination therapy of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient. See Derossi et al. (1998), Trends Cell Biol. 8, 84-87.

Preferably, the combination therapy of the invention is a unit dosage containing a daily dose or unit, daily sub-dose or an appropriate fraction thereof, of the active ingredient.

The combination therapy of the invention will normally be administered orally or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the combination therapy of the invention can be administered alone but will generally be administered in admixture with a suitable pharmaceutical excipient, diluent or carrier selected with regard to the intended route of administration and standard pharmaceutical practice.

For example, the combination therapy of the invention can be administered orally, buccally or sublingually in the form of tablets, capsules, ovules, elixirs, solutions or suspensions, which may contain flavouring or colouring agents, for immediate-, delayed- or controlled-release applications. The agents, medicaments and pharmaceutical compositions of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the agents, medicaments and pharmaceutical compositions of the invention may be combined with various sweetening or flavouring agents, colouring matter or dyes, with emulsifying and/or suspending agents and with diluents such as water, ethanol, propylene glycol and glycerin, and combinations thereof.

The combination therapy of the invention can be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

The combination therapy of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an agent of the invention and a suitable powder base such as lactose or starch.

Aerosol or dry powder formulations are preferably arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the combination therapy of the invention can be administered in the form of a suppository or pessary, or they may be applied topically in the form of a lotion, solution, cream, gel, ointment or dusting powder. The agents, medicaments and pharmaceutical compositions of the invention may also be transdermally administered, for example, by the use of a skin patch. They may also be administered by the ocular route, particularly for treating diseases of the eye.

For ophthalmic use, the combination therapy of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the combination therapy of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, local administration of the combination therapy of the invention at or near the site of a tumour is the preferred route, in particular intra-tumoural or peri-tumoural administration.

The pharmaceutical compositions will be administered to a patient in a pharmaceutically effective dose/a therapeutically effective amount, as described above. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period. The antibody polypeptides can be formulated at various concentrations, depending on the efficacy/toxicity of the polypeptide being used. For example, the formulation may comprise the active antibody polypeptide at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active antibody polypeptide at a concentration of between 300 μM and 700 μM.

Typically, the therapeutic dose of the antibody polypeptide (with or without a therapeutic moiety) in a human patient will be in the range of 100 μg to 1 g per administration (based on a body weight of 70 kg, e.g. between 300 μg to 700 mg per administration). For example, the maximum therapeutic dose may be in the range of 0.1 to 10 mg/kg per administration, e.g. between land 10 mg/kg or between 0.1 and 5 mg/kg or between 1 and 5 mg/kg or between 0.1 and 2 mg/kg. Most preferably, the therapeutic dose is between 1 and 10 mg/kg, optionally between 2.5 and 7.5 mg/kg. It will be appreciated that such a dose may be administered at different intervals, as determined by the oncologist/physician; for example, a dose may be administered daily, twice-weekly, weekly, bi-weekly or monthly.

It will be further appreciated by persons skilled in the art that the polypeptides and pharmaceutical formulations of the present invention have utility in both the medical and veterinary fields. Thus, the methods of the invention may be used in the treatment of both human and non-human animals (such as horses, dogs and cats). Preferably, however, the patient is human.

For veterinary use, the combination therapy of the invention is administered as a suitably acceptable formulation in accordance with normal veterinary practice and the veterinary surgeon will determine the dosing regimen and route of administration which will be most appropriate for a particular animal.

The invention also provides a kit for treating a solid tumour in a subject, the kit comprising (a) a therapeutically effective amount of an antibody that specifically binds to CD137 and (b) a therapeutically effective amount of an further immunotherapeutic agent that is suitable for systemic administration to a subject. The further immunotherapeutic agent is a PD-1-inhibitor, optionally as defined in the first aspect. The antibody that specifically binds to CD137 is preferably provided in a form suitable for local administration to a tumour.

### Nucleic Acids, Vectors and Hosts

The invention also provides a kit comprising a first isolated nucleic acid molecule encoding an antibody or antigen-binding fragment thereof that specifically binds to CD137 (or a component polypeptide chain thereof) and:
(i) a PD-1 inhibitor, and/or
(ii) a second isolated nucleic acid molecule encoding an antibody of antigen-binding fragment thereof that specifically binds to PD-1 or PD-1 (or a component polypeptide chain thereof).

By “° nucleic acid molecule” we include DNA (e.g. genomic DNA or complementary DNA) and mRNA molecules, which may be single- or double-stranded. By “isolated” we mean that the nucleic acid molecule is not located or otherwise provided within a cell.

In one embodiment, the first and/or second nucleic acid molecule(s) is/are cDNA molecule(s).

In an embodiment, the first and/or second isolated nucleic acid molecules encode an antibody heavy chain or variable region thereof and/or encode an antibody light chain or variable region thereof.

Preferably, the first nucleic acid molecule comprises one or more nucleotide sequence selected from either SEQ ID NO: 9 and/or SEQ ID NO: 10, reproduced below.

Nucleotide sequence encoding VH region of “1630”
[SEQ ID NO: 9]
GAGGTGCAGCTGTTGGAGAGCGGGGGAGGCTTGGTACAGCCTGGGGGG
TCCCTGCGCCTCTCCTGTGCAGCCAGCGGATTCACCTTTGGTTACTCT
TACATGTCTTGGGTCCGCCAGGCTCCAGGGAAGGGGCTGGAGTGGGTC
TCATCTATTGGTTCTGGTTCTTCTTACACATACTATGCAGACTCCGTG
AAGGGCCGGTTCACCATCTCCCGTGACAATTCCAAGAACACGCTGTAT
CTGCAAATGAACAGCCTGCGTGCCGAGGACACGGCTGTATATTATTGT
GCGCGCGTTTACTCTTCTCCGGGTATTGACTATTGGGGCCAGGGAACC
CTGGTCACCGTCTCCTCA
Nucleotide sequence encoding VL region of “1631”
[SEQ ID NO: 10]
GACATCCAGATGACCCAGTCTCCATCCTCCCTGAGCGCATCTGTAGGA
GACCGCGTCACCATCACTTGCCGGGCAAGTCAGAGCATTAGCAGCTAT
TTAAATTGGTATCAGCAGAAACCAGGGAAAGCCCCTAAGCTCCTGATC
TATGCTGCATCCAGTTTGCAAAGTGGGGTCCCATCACGTTTCAGTGGC
AGTGGAAGCGGGACAGATTTCACTCTCACCATCAGCAGTCTGCAACCT
GAAGATTTTGCAACTTATTACTGTCAACAGTACTACACTTGGGTTCCG
TTCACTTTTGGCCAGGGGACCAAGCTGGAGATCAAA

In an alternative preferred embodiment, the first nucleic acid molecule comprises one or more nucleotide sequence selected from either SEQ ID NO: 27 and/or SEQ ID NO: 28, reproduced below.

Nucleotide sequence encoding VH region of “2674”
[SEQ ID NO: 27]
gaggtgcagttgttggaatctggcggaggattggtgcagcctggcgga
tctctgagactgtcttgtgccgcctctggcttcaacttcggctactcc
tacatgtcctgggtccgacaggctcctggcaaaggactggaatgggtg
tcctccatcggctccaccagctctcacacctactacgccgattccgtg
aagggcagattcaccatcagccgggacaactccaagaacaccctgtac
ctgcagatgaactccctgagagccgaggacaccgccgtgtactactgt
gccagagtgtactcctctcctggcatcgattattggggccagggcaca
ctggtcaccgtgtcctctgcttctaccaagggaccctctgtgttccct
ctggctccttgctccagatccacctctgagtctaccgctgctctgggc
tgcctggtcaaggattactttcctgagcctgtgaccgtgtcttggaac
tccggtgctctgacatccggcgtgcacacatttccagctgtgctgcag
tcctccggcctgtactctctgtcctctgtcgtgaccgtgccttctagc
tctctgggcaccaagacctacacctgtaacgtggaccacaagccttcc
aacaccaaggtggacaagcgcgtggaatctaagtacggccctccatgt
ccaccatgtcctgctccagaattcctcggcggaccaagcgtgttcctg
tttcctccaaagcctaaggacaccctgatgatctctcggacccctgaa
gtgacctgcgtggtggtggatgtgtctcaagaggacccagaagtgcag
ttcaattggtacgtggacggcgtggaagtgcacaacgccaagaccaag
cctagagaggaacagttcaactccacctacagagtggtgtccgtgctg
accgtgctgcaccaggattggctgaacggcaaagagtacaagtgcaag
gtgtccaacaagggcctgccttccagcatcgaaaagaccatctccaag
gctaagggccagcctcgggaacctcaggtttacaccctgcctccaagc
caagaggaaatgaccaagaaccaggtgtccctgacctgcctcgtgaag
ggattctacccttccgatatcgccgtggaatgggagtctaacggccag
ccagagaacaactacaagacaacccctcctgtgctggactccgacggc
tctttcttcctgtattctcgcctgaccgtggacaagtctcggtggcaa
gagggcaacgtgttctcctgctctgtgatgcacgaggccctgcacaac
cactacacacagaagtccctgtctctgtccctgggcaag
Nucleotide sequence encoding VL region of “2675”
[SEQ ID NO: 28]
gacatccagatgacccagtctccatcctctctgtctgcctctgtgggc
gacagagtgaccatcacctgtcgggcttctcagtccatcggcagcacc
ctgaactggtatcagcagaagcctggcaaggcccctaagctgctgatc
tatggcgctagctctctgcagtctggcgtgccctctagattttccggc
tctggctctggcaccgacttcaccctgacaatcagttccctgcagcct
gaggacttcgccacctactactgccagcagtactacacctgggtgccc
tttacctttggccagggcaccaagctggaaatcaagagaaccgtggcc
gctccttccgtgttcatcttcccaccatctgacgagcagctgaagtcc
ggcacagcttctgtcgtgtgcctgctgaacaacttctaccctcgggaa
gccaaggtgcagtggaaggtggacaatgccctgcagtccggcaactcc
caagagtctgtgaccgagcaggactccaaggactctacctacagcctg
tcctccacactgaccctgtctaaggccgactacgagaagcacaaggtg
tacgcctgcgaagtgacccatcagggactgtctagccccgtgaccaag
tccttcaacagaggcgagtgt

It will be appreciated by persons skilled in the art that the first nucleic acid molecule may be codon-optimised for expression of the antibody polypeptide in a particular host cell, e.g. for expression in human cells (for example, see Angov, 2011, Biotechnol. J. 6(6):650-659, the disclosures of which are incorporated herein by reference).

The invention also provides a kit comprising a vector comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-1 or a component polypeptide chain thereof.

In an embodiment, the vector is an expression vector. The first and/or second isolated nucleic acid may be as described earlier.

The invention also provides a kit comprising a host cell comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof.

Preferably, the host cell may be a mammalian cell (e.g. a human cell, or Chinese hamster ovary cell, e.g. CHOK1SV cells), a bacterial cell or a yeast cell. The first and/or second isolated nucleic acid may be as described earlier. The first and/or second isolated nucleic acid may be comprised in a vector, such as an expression vector.

### Brief Description of the Sequence Listing

SEQ ID NO: 1 is the amino acid sequence of VH region of “1630”.

SEQ ID NO: 2 is the amino acid sequence of VL region of “1631”.

SEQ ID NO: 3 is the amino acid sequence of HCDR 1 of “1630”.

SEQ ID NO: 4 is the amino acid sequence of HCDR 2 of “1630”.

SEQ ID NO: 5 is the amino acid sequence of HCDR 3 of “1630”.

SEQ ID NO: 6 is the amino acid sequence of LCDR 1 of “1631”.

SEQ ID NO: 7 is the amino acid sequence of LCDR 2 of “1631”.

SEQ ID NO: 8 is the amino acid sequence of LCDR 3 of “1631”.

SEQ ID NO: 9 is the nucleotide sequence encoding VH region of “1630”.

SEQ ID NO: 10 is the nucleotide sequence encoding VL region of “1631”.

SEQ ID NO: 11 is the amino acid sequence of human CD137 sequence (amino acids 66 to 107 correspond to domain 2 of human CD137).

SEQ ID NO: 12 is the amino acid sequence of IgG1 heavy chain constant region.

SEQ ID NO: 13 is the amino acid sequence of modified IgG4 constant region.

SEQ ID NO: 14 is the amino acid sequence of modified IgG4 constant region.

SEQ ID NO: 15 is the amino acid sequence of wild-type IgG4 constant region.

SEQ ID NO: 16 is the amino acid sequence of kappa chain constant region.

SEQ ID NO: 17 is the full amino acid sequence of heavy chain of “1630”.

SEQ ID NO: 18 is the full amino acid sequence of the light chain of “1631”.

SEQ ID NO: 19 is the amino acid sequence of the VH region of “2674”.

SEQ ID NO: 20 is the amino acid sequence of the VL region of “2675”.

SEQ ID NO: 21 is the amino acid sequence of HCDR 1 of “2674”.

SEQ ID NO: 22 is the amino acid sequence of HCDR 2 of “2674”.

SEQ ID NO: 23 is the amino acid sequence of HCDR 3 of “2674”.

SEQ ID NO: 24 is the amino acid sequence of LCDR 1 of “2675”.

SEQ ID NO: 25 is the amino acid sequence of LCDR 2 of “2675”.

SEQ ID NO: 26 is the amino acid sequence of LCDR 3 of “2675”.

SEQ ID NO: 27 is the nucleotide sequence encoding VH region of “2674”.

SEQ ID NO: 28 is the nucleotide sequence encoding VL region of “2675”.

SEQ ID NO: 29 is the full amino acid sequence of the heavy chain “2674”.

SEQ ID NO: 30 is the full amino acid sequence of the light chain of “2675”.

SEQ ID NO: 31 is the heavy chain amino acid sequence of Nivolumab.

SEQ ID NO: 32 is the light chain amino acid sequence of Nivolumab.

SEQ ID NO: 33 is the heavy chain amino acid sequence of Pembrolizumab.

SEQ ID NO: 34 is the light chain amino acid sequence of Pembrolizumab.

SEQ ID NO: 35 is the amino acid sequence of the human PD-1 sequence.

SEQ ID NO: 36 is the amino acid sequence of the human PD-L1 sequence.

SEQ ID NO: 37 is the heavy chain sequence of Pidilizumab.

SEQ ID NO: 38 is the light chain sequence of Pidilizumab.

SEQ ID NO: 39 is the heavy chain sequence of Cemiplimab.

SEQ ID NO: 40 is the light chain sequence of Cemiplimab.

SEQ ID NO: 41 is the heavy chain sequence of Spartalizumab.

SEQ ID NO: 42 is the light chain sequence of Spartalizumab.

SEQ ID NO: 43 is the heavy chain sequence of Camrelizumab.

SEQ ID NO: 44 is the light chain sequence of Camrelizumab.

SEQ ID NO: 45 is the heavy chain sequence of Tislelizumab.

SEQ ID NO: 46 is the light chain sequence of Tislelizumab.

SEQ ID NO: 47 is the heavy chain sequence of Toripalimab.

SEQ ID NO: 48 is the light chain sequence of Toripalimab.

SEQ ID NO: 49 is the heavy chain sequence of Dostarlimab.

SEQ ID NO: 50 is the light chain sequence of Dostarlimab.

SEQ ID NO: 51 is the heavy chain sequence of INCMGA00012.

SEQ ID NO: 52 is the light chain sequence of INCMGA00012.

SEQ ID NO: 53 is the heavy chain sequence of Atezolizumab.

SEQ ID NO: 54 is the light chain sequence of Atezolizumab.

SEQ ID NO: 55 is the heavy chain sequence of Durvalumab.

SEQ ID NO: 56 is the light chain sequence of Durvalumab.

SEQ ID NO: 57 is the heavy chain sequence of Avelumab.

SEQ ID NO: 58 is the light chain sequence of Avelumab.

SEQ ID NO: 59 is the heavy chain sequence of CK-301.

SEQ ID NO: 60 is the light chain sequence of CK-301.

SEQ ID NO: 61 is the heavy chain sequence of JTX-4014.

SEQ ID NO: 62 is the light chain sequence of JTX-4014.

Embodiments of the invention include, but are not limited to, the following:
A. A method for treating a solid tumour in a subject, the method comprising (a) administering to the subject a therapeutically effective amount of an antibody, or antigen binding portion thereof, that specifically binds to CD137 (preferably that is retained at the tumour site following administration), and (b) systemically administering to the subject a therapeutically effective amount of an further immunotherapeutic agent, wherein the further immunotherapeutic agent is a PD-1 inhibitor.
B. The method according to Embodiment A wherein said PD-1 inhibitor is an anti-PD-1 or an anti-PD-L1 antibody.
C. The method according to any one of the preceding Embodiments wherein the solid tumour is an adenoma, a blastoma, a carcinoma, a desmoid tumour, a desmopolastic small round cell tumour, an endocrine tumour, a germ cell tumour, a lymphoma, a sarcoma, a Wilms tumour, a lung tumour, a colon tumour, a lymph tumour, a breast tumour or a melanoma.
D. The method according to any one of the preceding Embodiments wherein the solid tumour is a lung tumour (such as a non-small cell lung cancer or a small cell lung cancer); a head and/or neck tumour, a gastric tumour, oesophageal tumour, renal tumour, urothelial tumour, MSI-high tumour, dMMR tumour, TMB-high tumour, breast tumour, cervical tumour, prostate tumour, or a melanoma, preferably wherein the solid tumour is metastatic.
E. The method according to any one of the preceding Embodiments, wherein the antibody of step (a) comprises at least one CDR selected from SEQ ID NOs 3, 4, 5, 6, 7 and 8.
F. The method according to any one of the preceding Embodiments, wherein the antibody of step (a) comprises the CDR sequences of SEQ ID NOs: 3, 4 and 5 and/or SEQ ID NOs: 6, 7 and 8.
G. The method according to any one of the preceding Embodiments, wherein the antibody of step (a) comprises the light chain variable region of SEQ ID NO: 2 and/or the heavy chain variable region of SEQ ID NO: 1.
H. The method according to any one of the preceding Embodiments, wherein the antibody of step (a) comprises the light chain constant region of SEQ ID NO: 16 and/or the heavy chain constant region of SEQ ID NO: 13.
I. The method according to any one of the preceding Embodiments, wherein the antibody of step (a) competes for binding to human CD137 with an antibody which comprises the light chain variable region of SEQ ID NO: 2 and the heavy chain variable region of SEQ ID NO: 1.
J. The method according to any one of the preceding Embodiments, wherein the antibody of step (a) comprises at least one CDR selected from 21, 22, 23, 24, 25 and 26, optionally wherein the antibody of step (a) comprises the CDR sequences of SEQ ID NOs: 21, 22 and 23 and/or SEQ ID NOs: 24, 25 and 26.
K. The method according to any one of the preceding Embodiments, wherein the antibody of step (a) comprises the light chain variable region of SEQ ID NO: 20 and/or the heavy chain variable region of SEQ ID NO: 19.
L. The method according to any one of the preceding Embodiments, wherein the antibody of step (a) comprises the light chain constant region of SEQ ID NO: 16 and/or the heavy chain constant region of SEQ ID NO: 13.
M. The method according to any one of the preceding Embodiments, wherein the antibody of step (a) competes for binding to human CD137 with an antibody which comprises the light chain variable region of SEQ ID NO: 20 and the heavy chain variable region of SEQ ID NO: 19.
N. The method according to any one of the preceding Embodiments wherein steps (a) and (b) are carried out simultaneously or wherein step (b) is carried out within 24 hours after step (a), between 24 hours and two weeks after step (a), between 24 hours and one week after step (a), between 24 and 72 hours after step (a), or between 24 and 48 hours after step (a), preferably wherein step (b) is carried out within 24 hours after step (a).
O. The method according to any one of the preceding Embodiments wherein step (a) comprises systemic administration of the antibody to the subject, optionally wherein the antibody is formulated as a composition suitable for system administration with at least one pharmaceutically acceptable diluent or carrier.
P. The method according to any one of the preceding Embodiments, wherein at least 30% of the amount of antibody administered in step (a) is retained at the tumour site at four hours after administration, preferably wherein at least 40% of the said amount is retained at the tumour site at four hours after administration.
Q. The method according to any one of the preceding Embodiments wherein the further immunotherapeutic agent of step (b) is formulated as a composition suitable for systemic administration with at least one pharmaceutically acceptable diluent or carrier.
R. The method according to one of the preceding Embodiments wherein step (a) is conducted on multiple separate occasions and step (b) is conducted such that exposure of the subject to the further immunotherapeutic agent is continuous for the duration of the method.
S. The method according to any one of the preceding Embodiments wherein the subject is a human.
T. A kit for treating a solid tumour in a subject, the kit comprising (a) a therapeutically effective amount of an antibody that specifically binds to CD137 and preferably is retained at the tumour site following administration and optionally (b) a therapeutically effective amount of a further immunotherapeutic agent that is suitable for systemic administration to a subject, wherein the further immunotherapeutic agent is a PD-1 inhibitor.
U. An antibody, or antigen binding portion thereof, that specifically binds to CD137 and preferably is capable of being retained at the tumour site following administration, for use in treating cancer, such as a solid tumour, in a subject in combination with one or more further immunotherapeutic agents, wherein the one or more further immunotherapeutic agent(s) is a PD-1 inhibitor.
V. An antibody, or antigen binding portion thereof, according to Embodiment U wherein said PD-1 inhibitor is an anti-PD-1 or an anti-PD-L1 antibody.
W. An antibody, or antigen binding portion thereof, according to any of Embodiments U or V wherein the solid tumour is an adenoma, a blastoma, a carcinoma, a desmoid tumour, a desmopolastic small round cell tumour, an endocrine tumour, a germ cell tumour, a lymphoma, a sarcoma, a Wilms tumour, a lung tumour, a colon tumour, a lymph tumour, a breast tumour or a melanoma.
X. An antibody, or antigen binding portion thereof, according to any one of Embodiments U to W wherein the solid tumour is a lung tumour (such as a non-small cell lung cancer or a small cell lung cancer); a head and/or neck tumour, a gastric tumour, oesophageal tumour, renal tumour, urothelial tumour, MSI-high tumour, dMMR tumour, TMB-high tumour, breast tumour, cervical tumour, prostate tumour, or a melanoma, preferably wherein the solid tumour is metastatic.
Y. An antibody, or antigen binding portion thereof, according to any one of Embodiments U to X comprising at least one CDR selected from SEQ ID NOs 3, 4, 5, 6, 7 and 8.
Z. An antibody, or antigen binding portion thereof, according to any one of Claims Embodiments U to Y comprising at least one CDR selected from SEQ ID NOs 21, 22, 23, 24, 25 and 26.
AA. An antibody, or antigen binding portion thereof, according to any one of Embodiments U to Z comprising the CDR sequences of SEQ ID NOs: 3, 4, 5, 6, 7 and 8 or SEQ ID NOs: 21, 22, 23, 24, 25 and 26.
BB. An antibody, or antigen binding portion thereof, according to any one of Embodiments U to AA comprising the light chain variable region of SEQ ID NO: 2 and/or the heavy chain variable region of SEQ ID NO: 1 or comprising the light chain variable region of SEQ ID NO: 20 and/or the heavy chain variable region of SEQ ID NO: 19.
CC. An antibody, or antigen binding portion thereof, according to any one of Embodiments U to BB comprising the light chain constant region of SEQ ID NO: 16 and/or the heavy chain constant region of SEQ ID NO: 13
DD. An antibody, or antigen binding portion thereof, according to any one of Embodiments U to CC wherein the antibody, or antigen binding portion thereof, competes for binding to human CD137 with an antibody which comprises the light chain variable region of SEQ ID NO: 1 and the heavy chain variable region of SEQ ID NO: 2 and/or competes for binding to human CD137 with an antibody which comprises the light chain variable region of SEQ ID NO: 19 and the heavy chain variable region of SEQ ID NO: 20.
EE. An antibody, or antigen binding portion thereof, according to any one of Embodiments U to DD wherein the antibody is formulated as a composition suitable for local administration with at least one pharmaceutically acceptable diluent or carrier, and wherein the composition comprises a PD-1 inhibitor.
FF. A combination therapy composition according to Embodiment EE wherein said PD-1 inhibitor is an anti-PD-1 or an anti-PD-L1 antibody, optionally as defined according to any aspect herein.
GG. Use of an antibody, or antigen binding portion thereof, as defined in any one of Embodiments U to DD in the preparation of a medicament for the treatment of a solid tumour in a subject.

It is to be understood that different applications of the disclosed combination therapies and methods may be tailored to the specific needs in the art. It is also to be understood that the terminology used herein is for the purpose of describing particular embodiments of the invention only, and is not intended to be limiting. Where a feature is described with reference to a specific aspect, it will be appreciated by the skilled person that said feature may also apply to other related aspects.

In addition as used in this specification and the appended claims, the singular forms “a”, “an”, and “the” include plural referents unless the content clearly dictates otherwise. Thus, for example, reference to “an antibody” includes “antibodies”, reference to “an antigen” includes two or more such antigens, reference to “a subject” includes two or more such subjects, and the like.

All publications, patents and patent applications cited herein, whether supra or infra, are hereby incorporated by reference in their entirety.

The listing or discussion of an apparently prior-published document in this specification should not necessarily be taken as an acknowledgement that the document is part of the state of the art or is common general knowledge.

The present invention is further illustrated by the following examples which should not be construed as further limiting. The contents of all figures and all references, patents and published patent applications cited throughout this application are expressly incorporated herein by reference.

### BRIEF DESCRIPTION OF THE FIGURES

Preferred, non-limiting examples which embody certain aspects of the invention will now be described, with reference to the following figures:

FIG. 1 shows the effect of administration of an anti-CD137 antibody (ATOR-1017) and/or a PD-1 inhibitor (RMP1-14 clone) on tumour volume in female h4-1BBtg mice infected with MC38 colon adenocarcinoma cells.

FIG. 2 shows the effect of administration of an anti-CD137 antibody (ATOR-1017) and/or a PD-1 inhibitor (RMP1-14 clone) on survival of female h4-1BBtg mice infected with MC38 colon adenocarcinoma cells.

FIG. 3 shows the effect of administration of an anti-CD137 antibody (ATOR-1017) and/or a PD-1 inhibitor (RMP1-14 clone) on tumour growth inhibition in female h4-1BBtg mice infected with MC38 colon adenocarcinoma cells.

FIG. 4 shows T cell activation by an anti-CD137 antibody (ATOR-1017) in combination with a PD-1 inhibitor (Nivolumab) in mixed lymphocyte reaction (MLR) assay.

FIG. 5 shows T cell activation by an anti-CD137 antibody (ATOR-1017) in combination with a PD-1 inhibitor (Pembrolizumab) in mixed lymphocyte reaction (MLR) assay.

FIG. 6 shows T cell activation by an anti-CD137 antibody (ATOR-1017) in combination with a PD-1 inhibitor (Nivolumab) in mixed lymphocyte reaction (MLR) assay.

FIG. 7 shows T cell activation by an anti-CD137 antibody (ATOR-1017) in combination with a PD-1 inhibitor (Atezolizumab) in mixed lymphocyte reaction (MLR) assay.

FIG. 8 shows T cell activation by an anti-CD137 antibody (ATOR-1017) in combination with a PD-1 inhibitor (Nivolumab) in mixed lymphocyte reaction (MLR) assay with exhausted CD4+ T cells.

### REFERENCES

Almeida J, Bueno C, Algueró M C et al. Comparative analysis of the morphological, cytochemical, immunophenotypical, and functional characteristics of normal human peripheral blood lineage(−)/CD16(+)/HLA-DR(+)/CD14(−/lo) cells, CD14(+) monocytes, and CD16(−) dendritic cells. Clin Immunol. 2001 September; 100(3):325-38.
Ascierto, P. A., Simeone, E., Sznol, M., Fu, Y. X., and Melero, I. (2010) Clinical experiences with anti-CD137 and anti-PD-1 therapeutic antibodies. Semin. Oncol. 37, 508-516.
Baessler T, Charton J E, Schmiedel B J, Grunebach F, Krusch M, Wacker A, Rammensee H G, Salih H R. CD137 ligand mediates opposite effects in human and mouse NK cells and impairs NK-cell reactivity against human acute myeloid leukemia cells. Blood. 2010 Apr. 15; 115(15):3058-69. doi: 10.1182/blood-2009-06-227934. Erratum in: Blood. 2010 Dec. 23; 116(26):6152. PubMed PMID: 20008791.
Bronte V, Brandau S, Chen S H et al. Recommendations for myeloid-derived suppressor cell nomenclature and characterization standards. Nat Commun. 2016 Jul. 6; 7:12150.
Bruhns P, Iannascoli B, England P et al. Specificity and affinity of human Fcgamma receptors and their polymorphic variants for human IgG subclasses. Blood. 2009 Apr. 16; 113(16):3716-25.
Cavnar M J, Zeng S, Kim T S et al. KIT oncogene inhibition drives intratumoral macrophage M2 polarization. J Exp Med. 2013 Dec. 16; 210(13):2873-86.
Cheeseman H M, Carias A M, Evans A B et al. Expression Profile of Human Fc Receptors in Mucosal Tissue: Implications for Antibody-Dependent Cellular Effector Functions Targeting HIV-1 Transmission. PLoS One. 2016 May 10; 11(5):e0154656.
Chester, C., Sanmamed, M. F., Wang, J. & Melero, I. Immunotherapy targeting 4-1BB: mechanistic rationale, clinical results, and future strategies. Blood. 2018. 131:49-57.
Chin M S et al. Structure of the 4-1BB/4-1BBL complex and distinct binding and functional properties of utomilumab and urelumab. Nat Commun. 2018; 9:4679.
Curran, M. A., Kim, M., Montalvo, W., Al-Shamkhani, A., and Allison, J. P. (2011) Combination CTLA-4 blockade and 4-1BB activation enhances tumour rejection by increasing T-cell infiltration, proliferation, and cytokine production. PLoS. ONE. 6, e19499.
Dubrot, J., Milheiro, F., Alfaro, C., Palazon, A., Martinez-Forero, I., Perez-Gracia, J. L., Morales-Kastresana, A., Romero-Trevejo, J. L., Ochoa, M. C., Hervas-Stubbs, S., Prieto, J., Jure-Kunkel, M., Chen, L., and Melero, I. (2010) Treatment with anti-CD137 mAbs causes intense accumulations of liver T cells without selective antitumour immunotherapeutic effects in this organ. Cancer Immunol. Immunother. 59, 1223-1233.
Gauttier, V., Judor, J. P., Le, G., V, Cany, J., Ferry, N., and Conchon, S. (2014) Agonistic anti-CD137 antibody treatment leads to antitumour response in mice with liver cancer. Int. J. Cancer 135, 2857-2867.
Gray, J. C., French, R. R., James, S., Al-Shamkhani, A., Johnson, P. W., and Glennie, M. J. (2008) Optimising anti-tumour CD8 T-cell responses using combinations of immunomodulatory antibodies. Eur. J. Immunol. 38, 2499-2511.
Guilliams M, Bruhns P, Saeys Y et al. The function of Fcγ receptors in dendritic cells and macrophages. Nat Rev Immunol. 2014 February; 14(2):94-108.
Guo, Z., Cheng, D., Xia, Z., Luan, M., Wu, L., Wang, G., and Zhang, S. (2013) Combined TIM-3 blockade and CD137 activation affords the long-term protection in a murine model of ovarian cancer. J. Transl. Med. 11, 215.
Eigenmann et al. Interstitial IgG antibody pharmacokinetics assessed by combined in vivo- and physiologically-based pharmacokinetic modelling approaches. J Physiol. 2017, 595(24): 7311-7330.
Elliott L A, Doherty G A, Sheahan K et al. Human Tumor-Infiltrating Myeloid Cells: Phenotypic and Functional Diversity. Front Immunol. 2017 Feb. 6; 8:86
Eruslanov E B, Bhojnagarwala P S, Quatromoni J G et al. Tumor-associated neutrophils stimulate T cell responses in early-stage human lung cancer. J Clin Invest. 2014 December; 124(12):5466-80.
Eruslanov E, Neuberger M, Daurkin I et al. Circulating and tumor-infiltrating myeloid cell subsets in patients with bladder cancer. Int J Cancer. 2012 Mar. 1; 130(5):1109-19.
Grugan K D, McCabe F L, Kinder M et al. Tumor-associated macrophages promote invasion while retaining Fc-dependent anti-tumor function. J Immunol. 2012 Dec. 1; 189(11):5457-66.
Hansen B D, Schmidt H, von der Maase H et al. Tumour-associated macrophages are related to progression in patients with metastatic melanoma following interleukin-2 based immunotherapy. Acta Oncol. 2006; 45(4):400-5.
Hogarth P M, Pietersz G A. Fc receptor-targeted therapies for the treatment of inflammation, cancer and beyond. Nat Rev Drug Discov. 2012 Mar. 30; 11(4):311-31.
Horton H M, Bernett M J, Peipp M, Pong E, Karki S, Chu S Y, Richards J O, Chen H, Repp R, Desjarlais J R, Zhukovsky E A. Fc-engineered anti-CD40 antibody enhances multiple effector functions and exhibits potent in vitro and in vivo antitumour activity against hematologic malignancies. Blood. 2010 Oct. 21; 116(16):3004-12. doi: 10.1182/blood-2010-01-265280. PubMed PMID: 20616215.
Hruz T, Laule O, Szabo G, Wessendorp F, Bleuler S, Oertle L, Widmayer P, Gruissem W, Zimmermann P: Genevestigator v3: a reference expression database for the meta-analysis of transcriptomes. Adv Bioinformatics 2008, 2008:420747.
Hu W, Li X, Zhang C et al. Tumor-associated macrophages in cancers. Clin Transl Oncol. 2016 March; 18(3):251-8.
Jolliff C R et al. (1982) Reference intervals for serum IgG, IgA, IgM, C3, and C4 as determined by rate nephelometry. Clin Chem 28: 126-128.
Kim, J. A., Averbook, B. J., Chambers, K., Rothchild, K., Kjaergaard, J., Papay, R., and Shu, S. (2001) Divergent effects of 4-1BB antibodies on antitumour immunity and on tumour-reactive T-cell generation. Cancer Res 61, 2031-2037.
Kluger H M et al. Defining tumor resistance to PD-1 pathway blockade: recommendations from the first meeting of the SITC Immunotherapy Resistance Taskforce. J Immunother Cancer. 2020, 8(1):e000398.
Kwong, B., Gai, S. A., Elkhader, J., Wittrup, K. D., and Irvine, D. J. (2013) Localized immunotherapy via liposome-anchored Anti-CD137+IL-2 prevents lethal toxicity and elicits local and systemic antitumour immunity. Cancer Res. 73, 1547-1558.
Lee, H. W., Park, S. J., Choi, B. K., Kim, H. H., Nam, K. O., and Kwon, B. S. (2002) 4-1BB promotes the survival of CD8+ T lymphocytes by increasing expression of Bcl-xL and Bfl-1. J Immunol 169, 4882-4888.
Lee, S. J., Myers, L., Muralimohan, G., Dai, J., Qiao, Y., Li, Z., Mittler, R. S., and Vella, A. T. (2004) 4-1BB and OX40 dual costimulation synergistically stimulate primary specific CD8 T cells for robust effector function. J. Immunol. 173, 3002-3012.
Li Y, Lee P Y, Kellner E S et al. Monocyte surface expression of Fcgamma receptor RI (CD64), a biomarker reflecting type-I interferon levels in systemic lupus erythematosus. Arthritis Res Ther. 2010; 12(3):R90
Li C, Luo X, Lin Y et al. A Higher Frequency of CD14+CD169+ Monocytes/Macrophages in Patients with Colorectal Cancer. PLoS One. 2015 Oct. 28; 10(10):e0141817.
Li, F. and Ravetch, J. V. (2011) Inhibitory Fcgamma receptor engagement drives adjuvant and anti-tumour activities of agonistic CD40 antibodies. Science 333, 1030-1034.
Liu et al. Development of a unique anti-CD137 therapeutic antibody: Efficacy and safety profiles in mono and combination therapy. J Clin Onc. 35(15):e23079.
Lu J, Chu J, Zou Z et al. Structure of FcγRI in complex with Fc reveals the importance of glycan recognition for high-affinity IgG binding. Proc Natl Acad Sci USA. 2015 Jan. 20; 112(3):833-8
McMillin, D. W., Hewes, B., Gangadharan, B., Archer, D. R., Mittler, R. S., and Spencer, H. T. (2006) Complete regression of large solid tumours using engineered drug-resistant hematopoietic cells and anti-CD137 immunotherapy. Hum. Gene Ther 17, 798-806.
Melero, I., Shuford, W. W., Newby, S. A., Aruffo, A., Ledbetter, J. A., Hellstrom, K. E., Mittler, R. S., and Chen, L. (1997) Monoclonal antibodies against the 4-1BB T-cell activation molecule eradicate established tumours. Nat Med 3, 682-685.
Melero I, Daniel Hirschhorn-Cymerman, Aizea Morales-Kastresana, et al. Agonist Antibodies to TNFR Molecules That Costimulate T and NK cells Clin Cancer Res 2013; 19:1044-1053. Published online Mar. 3, 2013.
Melero I, Antonio M. Grimaldi, Jose L. Perez-Gracia, et al. Clinical Development of Immunostimulatory Monoclonal Antibodies and Opportunities for Combination. Clin Cancer Res 2013; 19:997-1008.
Miller, R. E., Jones, J., Le, T., Whitmore, J., Boiani, N., Gliniak, B., and Lynch, D. H. (2002) 4-1BB-specific monoclonal antibody promotes the generation of tumour-specific immune responses by direct activation of CD8 T cells in a CD40-dependent manner. J Immunol 169, 1792-1800.
Morales-Kastresana, A., Sanmamed, M. F., Rodriguez, I., Palazon, A., Martinez-Forero, I., Labiano, S., Hervas-Stubbs, S., Sangro, B., Ochoa, C., Rouzaut, A., Azpilikueta, A., Bolanos, E., Jure-Kunkel, M., Gutgemann, I., and Melero, I. (2013) Combined immunostimulatory monoclonal antibodies extend survival in an aggressive transgenic hepatocellular carcinoma mouse model. Clin. Cancer Res. 19, 6151-6162.
Morimura T, Neuchrist C, Kitz K et al. Monocyte subpopulations in human gliomas: expression of Fc and complement receptors and correlation with tumor proliferation. Acta Neuropathol. 1990; 80(3):287-94.
Norton S E, Dunn E T, McCall J L et al. Gut macrophage phenotype is dependent on the tumor microenvironment in colorectal cancer. Clin Transl Immunology. 2016 Apr. 29; 5(4):e76.
Niu, L., Strahotin, S., Hewes, B., Zhang, B., Zhang, Y., Archer, D., Spencer, T., Dillehay, D., Kwon, B., Chen, L., Vella, A. T., and Mittler, R. S. (2007) Cytokine-mediated disruption of lymphocyte trafficking, hemopolesis, and induction of lymphopenia, anemia, and thrombocytopenia in anti-CD137-treated mice. J. Immunol. 178, 4194-4213.
Overdijk M B, Verploegen S, Ortiz Buijsse A, Vink T, Leusen J H, Bleeker W K, Parren P W. Crosstalk between human IgG isotypes and murine effector cells. J Immunol. 2012 Oct. 1; 189(7):3430-8. PubMed PMID: 22956577.
Palazón A, Ivan Martínez-Forero, Alvaro Teijeira, et al. The HIF-1a Hypoxia Response in Tumour-Infiltrating T Lymphocytes Induces Functional CD137 (4-1BB) for Immunotherapy Cancer Discovery 2012; 2:608-623. Published OnlineFirst Jun. 19, 2012.
Palazón A, Teijeira A, Martínez-Forero I, Hervás-Stubbs S, Roncal C, Peñuelas I, Dubrot J, Morales-Kastresana A, Perez-Gracia J L, Ochoa M C, Ochoa-Callejero L, Martínez A, Luque A, Dinchuk J, Rouzaut A, Jure-Kunkel M, Melero I. Agonist anti-CD137 mAb act on tumour endothelial cells to enhance recruitment of activated T lymphocytes. Cancer Res. 2011 Feb. 1; 71(3):801-11. doi:10.1158/0008-5472.CAN-10-1733. PubMed PMID: 21266358.
Pan, P. Y., Zang, Y., Weber, K., Meseck, M. L., and Chen, S. H. (2002) OX40 ligation enhances primary and memory cytotoxic T lymphocyte responses in an immunotherapy for hepatic colon metastases. Mol Ther 6, 528-536.
Porembka M R, Mitchem J B, Belt B A et al. Pancreatic adenocarcinoma induces bone marrow mobilization of myeloid-derived suppressor cells which promote primary tumor growth. Cancer Immunol Immunother. 2012 September; 61(9):1373-85.
Pulle, G., Vidric, M., and Watts, T. H. (2006) IL-15-dependent induction of 4-1BB promotes antigen-independent CD8 memory T cell survival. J Immunol 176, 2739-2748.
Rabu, C., Quemener, A., Jacques, Y., Echasserieau, K., Vusio, P., and Lang, F. (2005) Production of recombinant human trimeric CD137L (4-1BBL). Cross-linking is essential to its T cell co-stimulation activity. J Biol Chem 280, 41472-41481.
Ribas and Wolchock, Cancer immunotherapy using checkpoint blockade. Science. 2018, 359(6382):1350-1355.
Roussel M, Ferrell P B Jr, Greenplate A R et al. Mass cytometry deep phenotyping of human mononuclear phagocytes and myeloid-derived suppressor cells from human blood and bone marrow. J Leukoc Biol. 2017 August; 102(2):437-447
Sallin, M. A., Zhang, X., So, E. C., Burch, E., Cal, L., Lin, W., Chapoval, A. I., and Strome, S. E. (2014) The anti-lymphoma activities of anti-CD137 monoclonal antibodies are enhanced in FcgammaRIII(−/−) mice. Cancer Immunol. Immunother. 63, 947-958.
Sanmamed, M. F., Pastor, F., Rodriguez, A., Perez-Gracia, J. L., Rodriguez-Ruiz, M. E., Jure-Kunkel, M., and Melero, I. (2015) Agonists of Co-stimulation in Cancer Immunotherapy Directed Against CD137, OX40, GITR, CD27, CD28, and ICOS. Semin. Oncol. 42, 640-655.
Segal N H et al. Results from an Integrated Safety Analysis of Urelumab, an Agonist Anti-CD137 Monoclonal Antibody. Clin Cancer Res. 2017, 23(8):1929-1936.
Segal N H et al. Phase I Study of Single-Agent Utomilumab (PF-05082566), a 4-1BB/CD137 Agonist, in Patients with Advanced Cancer. Clin Cancer Res. 2018, 24(8):1816-1823.
Shuford, W. W., Klussman, K., Tritchler, D. D., Loo, D. T., Chalupny, J., Siadak, A. W., Brown, T. J., Emswiler, J., Raecho, H., Larsen, C. P., Pearson, T. C., Ledbetter, J. A., Aruffo, A., and Mittler, R. S. (1997) 4-1BB costimulatory signals preferentially induce CD8+ T cell proliferation and lead to the amplification in vivo of cytotoxic T cell responses. J Exp. Med 186, 47-55.
So, T., Lee, S. W., and Croft, M. (2008) Immune regulation and control of regulatory T cells by OX40 and 4-1BB. Cytokine Growth Factor Rev. 19, 253-262.
Solito S, Marigo I, Pinton L et al. Myeloid-derived suppressor cell heterogeneity in human cancers. Ann N Y Acad Sci. 2014 June; 1319:47-65.
Stewart R, Hammond S, Oberst M, Wilkinson R. (2014) The role of Fc gamma receptors in the activity of immunomodulatory antibodies for cancer. J Immunother. 2:29
St Rose, M. C., Taylor, R. A., Bandyopadhyay, S., Qui, H. Z., Hagymasi, A. T., Vella, A. T., and Adler, A. J. (2013) CD134/CD137 dual costimulation-elicited IFN-gamma maximizes effector T-cell function but limits Treg expansion. Immunol. Cell Biol. 91, 173-183.
Sun Y, Subudhi S K, Fu Y X. Co-stimulation agonists as a new immunotherapy for autoimmune diseases. Trends Mol Med. 2003 November; 9(11):483-9. Review. PubMed PMID:14604826.
Taraban, V. Y., Rowley, T. F., O'Brien, L., Chan, H. T., Haswell, L. E., Green, M. H., Tutt, A. L., Glennie, M. J., and Al-Shamkhani, A. (2002) Expression and costimulatory effects of the TNF receptor superfamily members CD134 (OX40) and CD137 (4-1BB), and their role in the generation of anti-tumour immune responses. Eur J Immunol 32, 3617-3627.
Tolcher A W et al. Phase Ib Study of Utomilumab (PF-05082566), a 4-1BB/CD137 Agonist, in Combination with Pembrolizumab (MK-3475) in Patients with Advanced Solid Tumor. Clin Cancer Res. 2017, 23(18):5349-5357.
Uno, T., Takeda, K., Kojima, Y., Yoshizawa, H., Akiba, H., Mittler, R. S., Gejyo, F., Okumura, K., Yagita, H., and Smyth, M. J. (2006) Eradication of established tumours in mice by a combination antibody-based therapy. Nat. Med. 12, 693-698.
Vidarsson G, Dekkers G, Rispens T. IgG subclasses and allotypes: from structure to effector functions. Front Immunol. 2014 Oct. 20; 5:520. doi:10.3389/fimmu.2014.00520. Review. PubMed PMID: 25368619; PubMed Central PMCID: PMC4202688.
Vinay, D. S. and Kwon, B. S. (2012) Immunotherapy of cancer with 4-1BB. Mol. Cancer Ther. 11, 1062-1070.
Wang W, Erbe A K, Hank J A, Morris Z S, Sondel P M. NK Cell-Mediated Antibody-Dependent Cellular Cytotoxicity in Cancer Immunotherapy. Front Immunol. 2015 Jul. 27; 6:368. doi: 10.3389/fimmu.2015.00368. Review. PubMed PMID: 26284063; PubMed Central PMCID: PMC4515552.
Wei, H., Zhao, L., Li, W., Fan, K., Qian, W., Hou, S., Wang, H., Dai, M., Hellstrom, I., Hellstrom, K. E., and Guo, Y. (2013) Combinatorial PD-1 blockade and CD137 activation has therapeutic efficacy in murine cancer models and synergizes with cisplatin. PLoS. ONE. 8, e84927.
Westwood, J. A., Darcy, P. K., Guru, P. M., Sharkey, J., Pegram, H. J., Amos, S. M., Smyth, M. J., and Kershaw, M. H. (2010) Three agonist antibodies in combination with high-dose IL-2 eradicate orthotopic kidney cancer in mice. J. Transl. Med. 8, 42.
Westwood, J. A., Matthews, G. M., Shortt, J., Faulkner, D., Pegram, H. J., Duong, C. P., Chesi, M., Bergsagel, P. L., Sharp, L. L., Huhn, R. D., Darcy, P. K., Johnstone, R. W., and Kershaw, M. H. (2014a) Combination anti-CD137 and anti-CD40 antibody therapy in murine myc-driven hematological cancers. Leuk. Res. 38, 948-954.
White A L, Chan H T, French R R, Willoughby J, Mockridge C I, Roghanian A, Penfold C A, Booth S G, Dodhy A, Polak M E, Potter E A, Ardern-Jones M R, Verbeek J S, Johnson P W, Al-Shamkhani A, Cragg M S, Beers S A, Glennie M J. Conformation of the human immunoglobulin G2 hinge imparts superagonistic properties to immunostimulatory anticancer antibodies. Cancer Cell. 2015 Jan. 12; 27(1):138-48. doi:10.1016/j.ccell.2014.11.001. PubMed PMID: 25500122; PubMed Central PMCID:PMC4297290.
Westwood, J. A., Potdevin Hunnam, T. C., Pegram, H. J., Hicks, R. J., Darcy, P. K., and Kershaw, M. H. (2014b) Routes of delivery for CpG and anti-CD137 for the treatment of orthotopic kidney tumours in mice. PLoS. ONE. 9, e95847.
Wilcox, R. A., Flies, D. B., Zhu, G., Johnson, A. J., Tamada, K., Chapoval, A. I., Strome, S. E., Pease, L. R., and Chen, L. (2002) Provision of antigen and CD137 signaling breaks immunological ignorance, promoting regression of poorly immunogenic tumours. J Clin Invest 109, 651-659.
Wilson, N. S., Yang, B., Yang, A., Loeser, S., Marsters, S., Lawrence, D., Li, Y., Pitti, R., Totpal, K., Yee, S., Ross, S., Vernes, J. M., Lu, Y., Adams, C., Offringa, R., Kelley, B., Hymowitz, S., Daniel, D., Meng, G., and Ashkenazi, A. (2011b) An Fcgamma receptor-dependent mechanism drives antibody-mediated target-receptor signaling in cancer cells. Cancer Cell 19, 101-113.
Wyzgol, A., Muller, N., Fick, A., Munkel, S., Grigoleit, G. U., Pfizenmaier, K., and Wajant, H. (2009) Trimer stabilization, oligomerization, and antibody-mediated cell surface immobilization improve the activity of soluble trimers of CD27L, CD40L, 41BBL, and glucocorticoid-induced TNF receptor ligand. J Immunol 183, 1851-1861.
Yuan Y et al. Recent Advancements in the Mechanisms Underlying Resistance to PD-1/PD-L1 Blockade Immunotherapy. Cancers 2021, 13, 663.
Zhang, N., Sadun, R. E., Arias, R. S., Flanagan, M. L., Sachsman, S. M., Nien, Y. C., Khawli, L. A., Hu, P., and Epstein, A. L. (2007) Targeted and untargeted CD137L fusion proteins for the immunotherapy of experimental solid tumours. Clin. Cancer Res. 13, 2758-2767.
Zhang B, Wang Z, Wu L et al. Circulating and tumor-infiltrating myeloid-derived suppressor cells in patients with colorectal carcinoma. PLoS One. 2013; 8(2):e57114

### EXAMPLES

### Example 1: Anti-Tumor Efficacy of Anti-CD137 Antibody in Combination with a PD-1 Inhibitor in MC38 Colon Carcinoma Tumor-Bearing h4-1BB Transgenic Mice

ATOR-1017, a human anti-4-1BB (anti-CD137) agonist IgG4 antibody, triggers potent dose-dependent anti-tumor efficacy in MC38 colon carcinoma tumor-bearing mice, transgenic for human 4-1BB (h4-1BBtg). This is demonstrated by reduced tumor volume, improved tumour growth inhibition and induction of complete responders, as well as prolonged survival of mice.

The present inventors have surprisingly found that an anti-CD137 antibody (such as ATOR-1017) combination therapy with a PD-1 inhibitor (surrogate anti-PD-1 antibody clone RPM1-14) results in a further improved anti-tumor efficacy.

### Materials and Methods

Female h4-1BBtg mice, 8 weeks of age, were inoculated with 5×10⁵ MC38 colon adenocarcinoma cells subcutaneously (s.c.) in the right front flank in a volume of 100 μl. On day 7 post inoculation, mice were randomized into six study groups, and treated with the anti-CD137 antibody ATOR-1017 intraperitoneally (i.p.) given at two doses, 0.5 mg/kg or 5 mg/kg, with or without the additional treatment with 5 mg/kg surrogate anti-PD-1 antibody (clone RMP1-14; Cat no: BE0146 supplied by BioXCell, 39 Labombard Rd, Lebanon, NH 03766, USA). Controls received 5 mg/kg human IgG4 isotype control antibody. Treatments were given twice weekly for three weeks, summing up to six dosing occasions in total. Tumor growth was measured twice weekly, and mice sacrificed once tumor volumes approached the ethical limit of 3000 mm³.

### Results and Conclusions

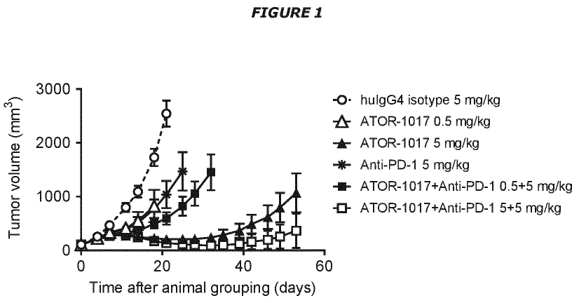

FIG. 1 demonstrates that treatment with the anti-CD137 antibody, ATOR-1017, given at 0.5 mg/kg results in a significantly reduced tumor volume compared to the isotype control (p=0.0015 on day 18), an effect that is more pronounced when ATOR-1017 is given at 5 mg/kg. In mice that receive either of the two doses of ATOR-1017 combined with anti-PD-1, the tumor volume is further reduced when compared to the corresponding ATOR-1017 monotherapy. This effect of the combination therapy is statistically significant in a one-tailed Mann-Whitney test for ATOR-1017 given at a dose of 5 mg/kg (p=0.0482 on day 35).

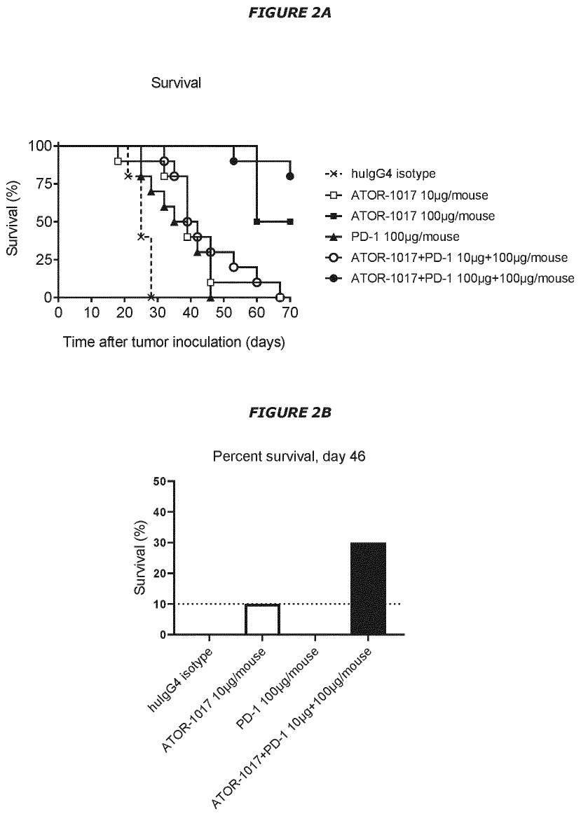

Treatment with the anti-CD137 antibody, ATOR-1017, at a dose of 10 and/or 100 μg/mouse or treatment with the surrogate anti-PD-1 antibody, clone RPM1-14, increased survival in female mice relative to the huIgG4 isotype control (FIG. 2A). In mice that receive a combination of ATOR-1017 and anti-PD-1, the survival rate is further increased when compared to the corresponding monotherapies (FIGS. 2A and 2B).

On day 46, the survival rate of mice receiving a combination therapy comprising 10 μg/mouse ATOR-1017 and 100 μg/mouse anti-PD-1 antibody was markedly increased in comparison to the individual monotherapies (FIG. 2B).

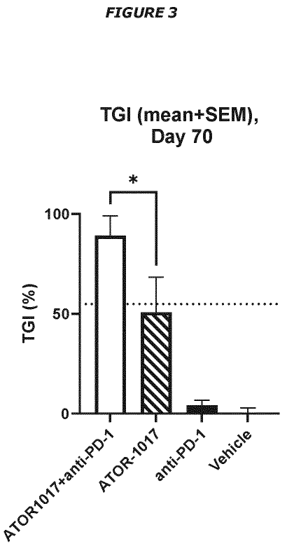

FIG. 3 demonstrates the effect of treatment with a combination of 100 μg/mouse anti-CD137 antibody, ATOR-1017, and 100 μg/mouse of a surrogate anti-PD-1 antibody, clone RPM1-14, on tumour growth inhibition (TGI, %) compared to the corresponding monotherapies. The inventors have surprisingly identified that the effect on TGI is further increased in mice receiving the combination therapy, compared to the corresponding ATOR-1017 monotherapy. The effect of the combination therapy is statistically significant in a T-test (p=0.0366) and demonstrates synergy between the two agents.

Furthermore, the number of complete responders was monitored (Table 3) and is increased in mice receiving the combination of ATOR-1017 and the anti-PD-1 antibody.

TABLE 3
Number of complete responders

| | Complete Responders (CRs) |
|---|---|
| Vehicle | 0 |
| PD-1 | 0 |
| ATOR-1017 | 5 |
| ATOR-1017 + PD-1 | 8 |

Overall, the anti-cancer effect of the combination of treatments targeting CD137 and inhibitors of PD-1 results is increased in comparison to treatment by monotherapy alone. In conclusion, these data show the unexpected effectiveness of a combination therapy with ATOR-1017 and anti-PD-1 antibody in the treatment of cancer.

### Example 2A: T Cell Activation of ATOR-1017 in Combination with PD-1 Inhibitor in Mixed Lymphocyte Reaction (MLR) Assay

### Background and Aim

To demonstrate the synergistic effect on T cell activation of combining ATOR-1017 with PD-1 inhibitors, T cell activation was assessed in a mixed lymphocyte reaction (MLR) using human primary CD4+ T cells and mature monocyte derived dendritic cells (mMo-DCs), where both targets (4-1BB and PD-1) are endogenously expressed.

### Materials and Methods

### Generation of Expanded CD4+ T Cells:

Human primary CD4+ T cells were isolated from leukocyte concentrates using a Human CD4+ T Cell Isolation Kit (130-096-533, Miltenyi Biotec Ltd) according to the manufacturer's instructions. Dynabeads™ Human T-Activator CD3/CD28 (11131D, Gibco) were used in the presence of 50 IU/mL recombinant human IL-2 (202-IL, R&D) with 1:1 bead to cell ratio to expand cells for 7 days. Dynabeads were removed and CD4+ T cells were rested overnight with reduced 10 IU/mL recombinant human IL-2.

Differentiation of mMo-DCs:

Monocytes were isolated from human PBMCs using a Human CD14 Isolation Kit, (130-050-201, Miltenyi Biotec Ltd, UK) following the manufacturer's instructions. Monocytes were differentiated to monocyte derived DCs (Mo-DCs) using Mo-DC differentiation media containing IL-4 and GM-CSF (130-094-812, Miltenyl). Mo-DCs were further matured into mature Mo-DCs (mMo-DCs) using a cocktail of Il-1β (130-093-563, Miltenyl), IL-6 (130-095-352, Miltenyi), TNFα (130-094-023, Miltenyl) and PGE2 (P0409-1MG, Merck Millipore).

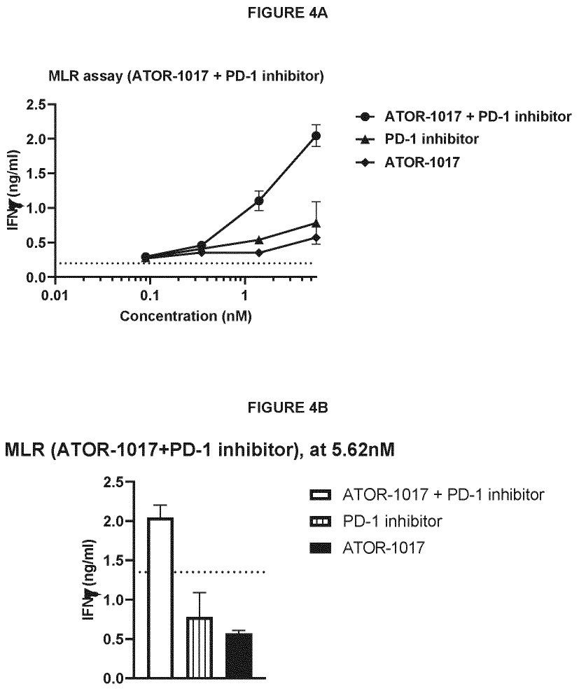

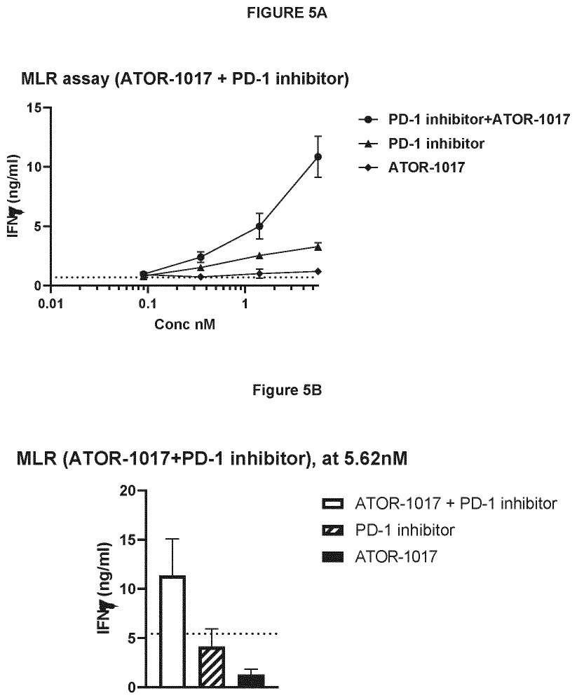

Titrations of ATOR-1017 and of PD-1 inhibitor in the presence of a fixed concentration of 45 nM of the F(ab)2 anti-Ig crosslinker (109-006-008, Jackson) were used to treat a 1:10 mix of mMo-DC cells and expanded CD4+ T cells for 7 days. Supernatants were analysed for interferon gamma (IFN-γ) using Monkey IFN gamma Elisa development Kit (3421M-1H-20, Mabtech). The PD-1 inhibitors tested by the present inventors include the anti-PD-1 antibody Nivolumab (Opdivo®, Bristol Myers Squibb) (shown in FIG. 4) and the anti-PD-1 antibody Pembrolizumab (Keytruda®, Merck) (shown FIG. 5).

Heavy chain sequence of Nivolumab (SEQ ID NO: 31):
QVQLVESGGG VVQPGRSLRL DCKASGITFS NSGMHWVRQA
PGKGLEWVAV IWYDGSKRYY ADSVKGRFTI SRDNSKNTLF
LQMNSLRAED TAVYYCATND DYWGQGTLVT VSSASTKGPS
VFPLAPCSRS TSESTAALGC LVKDYFPEPV TVSWNSGALT
SGVHTFPAVL QSSGLYSLSS VVTVPSSSLG TKTYTCNVDH
KPSNTKVDKR VESKYGPPCP PCPAPEFLGG PSVFLFPPKP
KDTLMISRTP EVTCVVVDVS QEDPEVQFNW YVDGVEVHNA
KTKPREEQFN STYRVVSVLT VLHQDWLNGK EYKCKVSNKG
LPSSIEKTIS KAKGQPREPQ VYTLPPSQEE MTKNQVSLTC
LVKGFYPSDI AVEWESNGQP ENNYKTTPPV LDSDGSFFLY
SRLTVDKSRW QEGNVFSCSV MHEALHNHYT QKSLSLSLGK
Light chain sequence of Nivolumab (SEQ ID NO: 32)
EIVLTQSPAT LSLSPGERAT LSCRASQSVS SYLAWYQQKP
GQAPRLLIYD ASNRATGIPA RFSGSGSGTD FTLTISSLEP
EDFAVYYCQQ SSNWPRTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC
Heavy chain sequence of Pembrolizumab
(SEQ ID NO: 33):
QVQLVQSGVE VKKPGASVKV SCKASGYTFT NYYMYWVRQA
PGQGLEWMGG INPSNGGTNF NEKFKNRVTL TTDSSTTTAY
MELKSLQFDD TAVYYCARRD YRFDMGFDYW GQGTTVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
Light chain sequence of Pembrolizumab
(SEQ ID NO: 34)
EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV
THQGLSSPVT KSFNRGEC

### Results and Conclusions

Both PD-1 inhibitors alone were able to activate the CD4+ T cells, whilst ATOR-1017 alone induced poor CD4+ T cell activation in the MLR assay (FIG. 4A, Nivolumab; FIG. 5A, Pembrolizumab). The dotted lines in FIGS. 4A and 5A shows the baseline level of allogeneic stimulation of the CD4+ T cells in the MLR assay (background level). However, the combined treatment of ATOR-1017 and PD-1 inhibitor synergistically improved the potency of the T cell activation compared to the individual monotherapies alone (FIGS. 4B and 5B). The dotted lines shown on FIGS. 4B and 5B indicates the additive effect of the ATOR-1017 and PD-1 inhibitor monotherapies.

In conclusion, these data show the unexpected effectives of a combination therapy with ATOR-1017 and a PD-1 inhibitor, such as an anti-PD-1 antibody, for T cell activation and treatment of cancer.

### Example 2B: T Cell Activation of ATOR-1017 in Combination with PD-1 Inhibitor in Mixed Lymphocyte Reaction (MLR) Assay

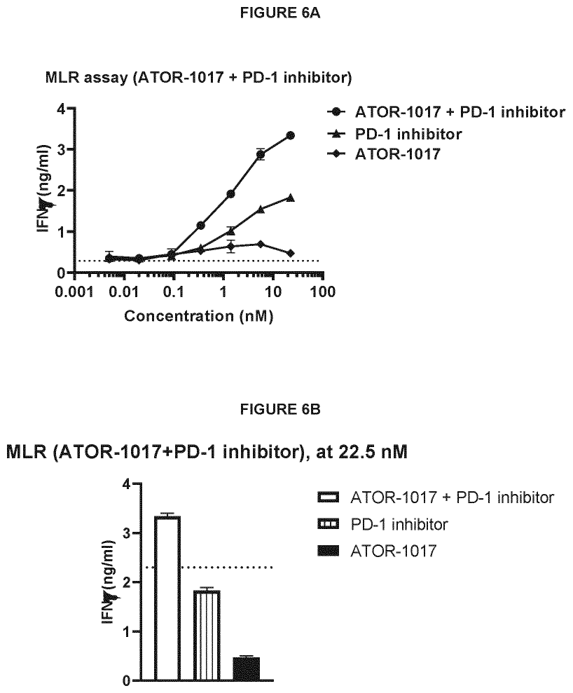

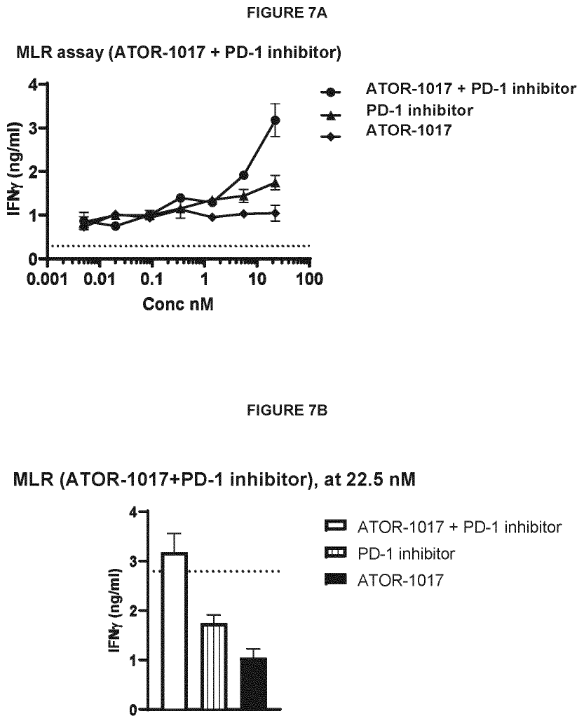

Example 2A was repeated, with the same materials and methods as described for Example 2A but using the anti-PD-1 antibody Nivolumab (FIG. 6) or the anti-PD-L1 antibody Atezolizumab (Tecentriq®, Roche) (FIG. 7) as the PD-1 inhibitor and using a variable concentration of the anti-F(ab)2 used for crosslinking. SEQ ID NO: 53 is the heavy chain sequence of Atezolizumab and SEQ ID NO: 54 is the light chain sequence of Atezolizumab. Anti-F(ab)2 was administered at a ratio of the ATOR-1017 dose (anti-F(ab)2/ATOR-1017 ratio of 5:1), instead of a fixed concentration of anti-F(ab)2. The combined treatment of ATOR-1017 and PD-1 inhibitor synergistically improved the potency of the T cell activation compared to the individual monotherapies alone (FIG. 6A, Nivolumab; FIG. 7A, Atezolizumab). The dotted lines in FIGS. 6A and 7A show the baseline level of allogeneic stimulation of the CD4+ T cells in the MLR assay (background level).

The dosages investigated for the monotherapy and combination studies were: 0.005, 0.02, 0.09, 0.35, 1.4, 5.62 and 22.5 nM. For a dosage of 5.62 nM for a monotherapy, the concentration of that monotherapy is 5.62 nM. For a dosage of 5.62 nM for a combination therapy each element (ATOR-1017 and the PD-1 inhibitor) is present at 5.62 nM.

A dose of 22.5 nM in the MLR assay corresponds approximately to a dose of 0.1 mg/kg when administered to a human patient.

A synergistic improvement of the combination therapy was observed at a high concentration (22.5 nM) as shown in FIG. 6B (Nivolumab) and FIG. 7B (Atezolizumab). The dotted lines shown on FIGS. 6B and 7B indicate the additive effect of the ATOR-1017 and PD-1 inhibitor monotherapies, demonstrating the synergistic effect of the combination.

### Example 3: Exhausted T Cell Activation of ATOR-1017 in Combination with Anti-PD-1 Antibody in Mixed Lymphocyte Reaction (MLR) Assay

### Background and Aim

To demonstrate the synergistic activity on exhausted T cell activation of combining ATOR-1017 with anti-PD-1. T cell activation was assessed in a mixed lymphocyte reaction (MLR) using human primary CD4+ T cells with an exhausted phenotype and mature monocyte derived dendritic cells (mMo-DCs), where both targets (4-1BB and PD-1) are endogenously expressed.

### Materials and Methods

### Generation of Exhausted CD4+ T Cells:

CD4+ T cells were expanded for 8 days as described for the generation of expanded CD4+ T cells previously, with the exception that every second day, for a total of 3 times during the 8 days expansion period, fresh CD3/CD28 Dynabeads were added to the CD4+ T cells. After 8 days, exhausted CD4+ T cells were characterized as having an increased expression of PD-1, TIM-3 and LAG-3 with flow cytometry.

Differentiation of mMo-DCs:

Mature mo-DC were generated as described previously for differentiation of mMo-DCs.

Titrations of ATOR-1017 and of a PD-1 inhibitor (Nivolumab) in the presence of F(ab)2 anti-Ig crosslinker (109-006-008, Jackson) were used to treat a 1:10 mix of mMo-DC cells and expanded CD4+ T cells for 7 days. Supernatants were analyzed for interferon gamma (IFN-γ) using Monkey IFN gamma Elisa development Kit (3421M-1H-20, Mabtech).

The dosages investigated for the monotherapy and combination studies were: 0.005, 0.02, 0.09, 0.35, 1.4, 5.62 and 22.5 nM. For a dosage of 5.62 nM for a monotherapy, the concentration of that monotherapy is 5.62 nM. For a dosage of 5.62 nM for a combination therapy each element (ATOR-1017 and the PD-1 inhibitor) is present at 5.62 nM.

A dose of 22.5 nM in the MLR assay corresponds approximately to a dose of 0.1 mg/kg when administered to a human patient.

### Results and Conclusions

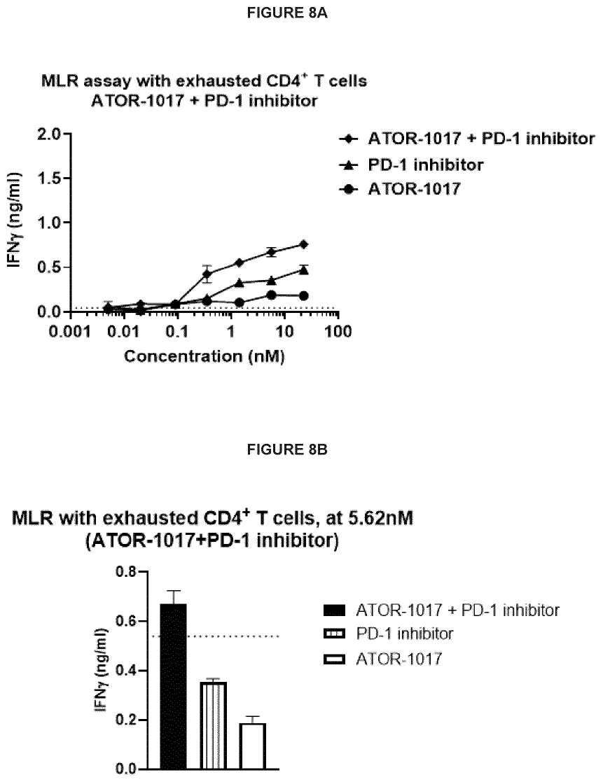

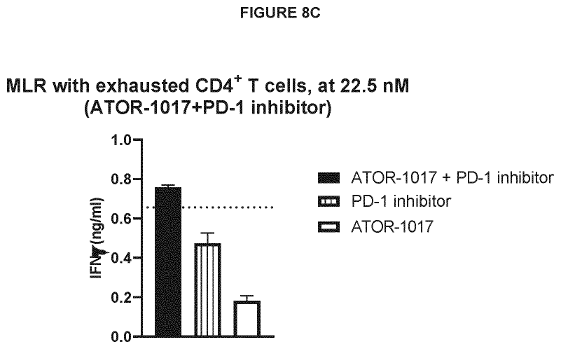

Exhausted CD4+ T cells are characterized as having an increased expression of PD-1, TIM-3 and LAG-3 as well as a reduced capacity to respond to allogenic stimuli. Anti-PD-1 alone was able to activate the exhausted CD4+ T cells while ATOR-1017 alone induced a poor CD4+ T cells activation in the MLR assay (FIG. 8A). However, the combined treatment of ATOR-1017 and a PD-1 inhibitor synergistically improved the potency of the exhausted CD4+ T cell activation compared to each monotherapy treatment alone (FIGS. 8B and 8C).

In conclusion, these data support a rationale for combination therapy with ATOR-1017 and PD-1 inhibitors (in particular PD-1/PD-L1 blocking antibodies) to activate exhausted T cell in cancer patients resulting in enhanced anti-tumor activity compared to monotherapy with either agent alone.

1. A combination therapy for use in the treatment or prevention of cancer in a subject comprising (a) an antibody, or antigen-binding fragment thereof, that specifically binds to CD137, and (b) a PD-1 inhibitor.

2. The combination therapy according to claim 1, wherein the cancer is a solid tumour.

3. The combination therapy according to any of the preceding claims wherein the cancer and/or solid tumour is selected from the groups consisting of prostate cancer; breast cancer; colorectal cancer; kidney cancer; pancreatic cancer; ovarian cancer; lung cancer; cervical cancer; rhabdomyosarcoma; neuroblastoma; bone cancer; multiple myeloma; leukemia (such as acute lymphoblastic leukemia [ALL] and acute myeloid leukemia [AML]), skin cancer (e.g. melanoma), bladder cancer and glioblastoma, an adenoma, a blastoma, a carcinoma, a desmoid tumour, a desmopolastic small round cell tumour, an endocrine tumour, a germ cell tumour, a lymphoma, a sarcoma, a Wilms tumour, a lung tumour, a colon tumour, a lymph tumour, a breast tumour and a melanoma.

4. The combination therapy to any one of the preceding claims wherein the cancer and/or solid tumour is a lung cancer (such as a non-small cell lung cancer (NSCLC) or a small cell lung cancer (SCLC)), a head and/or neck cancer, a gastric cancer, an oesophageal cancer, a renal cancer, a urothelial cancer, a melanoma, a mesothelioma, a breast cancer, a cervical cancer, a prostate cancer, a microsatellite instability (MSI)-high cancer, a cancer associated with DNA mismatch repair (dMMR) and/or a tumour mutational burden (TMB)-high cancer, preferably wherein the cancer and/or solid tumour is metastatic.

5. A combination therapy according to any of the preceding claims, wherein the antibody or antigen-binding fragment thereof that specifically binds to CD137:
a) has binding specificity for domain 2 of human CD137;
b) is a CD137 agonist; and/or
c) is capable of inhibiting the binding of reference antibody ‘1630/1631’ to human CD137,
optionally wherein the antibody or antigen binding fragment has binding specificity for domain 2 of human CD137; is a CD137 agonist; and is capable of inhibiting the binding of reference antibody ‘1630/1631’ to human CD137.

6. A combination therapy according to any of the preceding claims, wherein the antibody or antigen-binding fragment thereof that specifically binds to CD137:
a) has binding specificity for domain 2 of human CD137;
b) is a CD137 agonist; and/or
c) is capable of inhibiting the binding of reference antibody ‘2674/2675’ to human CD137,
optionally wherein the antibody or antigen binding fragment has binding specificity for domain 2 of human CD137; is a CD137 agonist; and is capable of inhibiting the binding of reference antibody ‘2674/2675’ to human CD137,

7. A combination therapy according to any of the preceding claims, wherein the antibody or antigen-binding fragment that specifically binds to CD137 exhibits one or more of the following properties:
a) the ability to stimulate CD137 and activate T cells and other immune cells via a cross-linking dependent mechanism; and/or
b) cross-reactivity with cyano-CD137 antibodies.

8. A combination therapy according to any of the preceding claims, wherein the antibody or antigen binding fragment thereof that specifically binds to CD137 is capable of binding an Fc receptor, optionally wherein the antibody or antigen binding fragment thereof that specifically binds to CD137 is capable of simultaneous binding to CD137 and an Fc receptor.

9. A combination therapy according to claim 8 wherein the ability of the antibody that specifically binds to CD137 to activate T cells is dependent upon binding to both CD137 and Fc receptors.

10. A combination therapy according to any of the preceding claims, wherein the antibody or antigen binding fragment thereof that specifically binds to CD137 is substantially incapable of inducing the following upon binding to cells expressing CD137:
a) antibody-dependent cellular cytotoxicity (ADCC);
b) antibody-dependent cellular phagocytosis (ADCP); and/or
c) complement-dependent cytotoxicity (CDC).

11. A combination therapy according to any of the preceding claim, wherein the antibody or antigen binding fragment thereof that specifically binds to CD137 is capable of inducing tumour immunity.

12. A combination therapy according to any of the preceding claims, wherein the antibody or antigen binding fragment thereof that specifically binds to CD137 is capable of binding to an epitope on the extracellular domain of CD137 which overlaps, at least in part, with the epitope on CD137 to which reference antibody 1630/1631 is capable of binding.

13. A combination therapy according to claim 12, wherein the antibody or antigen-binding fragment that specifically binds to CD137 is capable of binding to an epitope on the extracellular domain of CD137 which overlaps, at least in part, with the epitope on CD137 to which reference antibody 2674/2675 is capable of binding.

14. A combination therapy according to claim 12 or 13, wherein the epitope is located at or within amino acids 66 to 107 of human CD137.

15. A combination therapy according to any of the preceding claims wherein CD137 is localised on the surface of a cell.

16. A combination therapy according to any one of the preceding claims, wherein the antibody or antigen-binding fragment that specifically binds to CD137 comprises or consists of an intact antibody, for example an IgG1, IgG2, IgG3 or IgG4 antibody.

17. A combination therapy according to any one of the preceding claims, wherein the antibody or antigen-binding fragment thereof that specifically binds to CD137 comprises or consists of IgG4 antibody.

18. A combination therapy according to any one of claims 1 to 15, wherein the antibody or antigen-binding fragment thereof that specifically binds to CD137 comprises or consists of an antigen-binding fragment selected from the group consisting of Fv fragments (e.g. single chain Fv and disulphide-bonded Fv), Fab-like fragments (e.g. Fab fragments, Fab′ fragments and F(ab)₂ fragments) and domain antibodies (e.g. single VH variable domains or VL variable domains).

19. A combination therapy according to claim 18 wherein the antigen-binding fragment comprises or consists of an scFv.

20. A combination therapy according to any one of the preceding claims wherein the antibody or antigen-binding fragment thereof that specifically binds to CD137 is a recombinant polypeptide.

21. A combination therapy according to any one of the preceding claims wherein the antibody or antigen-binding fragment thereof that specifically binds to CD137 is monoclonal.

22. A combination therapy to any one of the preceding claims wherein the antibody or antigen-binding fragment thereof that specifically binds to CD137 is human or humanised.

23. A combination therapy according to any of the preceding claims, wherein the antibody or antigen-binding fragment thereof that specifically binds to CD137 comprises:
a) a heavy chain CDR1 sequence with the consensus sequence G, F, T/N, F, G, Y, S, Y;
b) a heavy chain CDR2 sequence with the consensus sequence I, G, S, G/T, S, S, Y/H, T; and
c) a heavy chain CDR3 sequence with the sequence ARVYSSPGIDY.

24. A combination therapy according to any of the preceding claims, wherein the antibody or antigen-binding fragment thereof that specifically binds to CD137 comprises:
a) a light chain CDR1 sequence with the consensus sequence Q, S, I, S/G, S, Y/T;
b) a light chain CDR2 sequence with the consensus sequence A/G, A, S; and
c) a light chain CDR3 sequence with the sequence QQYYTWVPFT.

25. A combination therapy according to any of the preceding claims, wherein the antibody or antigen-binding fragment thereof that specifically binds CD137 comprises a heavy chain variable region comprising the following CDRs:
a) GFTFGYSY [SEQ ID NO: 3] or an amino acid sequence containing up to 3 amino acid mutations compared to SEQ ID NO: 3, for example 1, 2 or 3 mutations;
b) IGSGSSYT [SEQ ID NO: 4] or an amino acid sequence containing up to 3 amino acid mutations compared to SEQ ID NO: 4, for example 1, 2 or 3 mutations; and
c) ARVYSSPGIDY [SEQ ID NO: 5] or an amino acid sequence containing up to 3 amino acid mutations compared to SEQ ID NO: 5, for example 1, 2 or 3 mutations.

26. A combination therapy according to any of the preceding claims, wherein the antibody or antigen-binding fragment thereof that specifically binds to CD137 comprises a heavy chain variable region comprising the CDRs of SEQ ID NOs 3, 4 and 5.

27. A combination therapy according to claim 26, wherein the antibody or antigen-binding fragment thereof that specifically binds to CD137 comprises a heavy chain variable region having the amino acid sequence of SEQ ID NO 1: or an amino acid sequence having at least 60% sequence identity therewith, for example at least 70%, 80%, 85%, 90%, 95%, 96%, 97%, 98% or 99% sequence identity.

28. A combination therapy according to any of the preceding claims, wherein the antibody or antigen-binding fragment thereof that specifically binds to CD137 comprises a light chain variable region comprising the following CDRs:
a) QSISSY [SEQ ID NO: 6] or an amino acid sequence containing up to 3 amino acid mutations compared to SEQ ID NO: 6, for example 1, 2 or 3 mutations;
b) AAS [SEQ ID NO: 7] or an amino acid sequence containing up to 2 amino acid mutations compared to SEQ ID NO: 7; for example 1 or 2 mutations; and
c) QQYYTWVPFT [SEQ ID NO: 8] or an amino acid sequence containing up to 3 amino acid mutations compared to SEQ ID NO: 8, for example 1, 2 or 3 mutations.

29. A combination therapy according to claim 28, wherein the antibody or antigen-binding fragment thereof that specifically binds to CD137 comprises a light chain variable region comprising the CDRs of SEQ ID NOs: 6, 7 and 8.

30. A combination therapy according to claim 29, wherein the antibody or antigen-binding fragment thereof that specifically binds to CD137 comprises a light chain variable region having the amino acid sequence of SEQ ID NO: 2 or an amino acid sequence having at least 60% sequence identity therewith, for example at least 70%, 80%, 85%, 90%, 95%, 96%, 97%, 98% or 99% sequence identity.

31. A combination therapy according to any of the preceding claims, wherein the antibody or antigen-binding fragment thereof that specifically binds to CD137 comprises the CDRs of SEQ ID NOs: 3, 4, 5, 6, 7 and 8.

32. A combination therapy according to any of the preceding claims, wherein the antibody or antigen-binding fragment thereof that specifically binds to CD137 comprises a heavy chain variable region which comprises or consists of the amino acid sequence of SEQ ID NO: 1 and a light chain variable region which comprises or consists of the amino acid sequence of SEQ ID NO: 2.

33. A combination therapy according to any of claims 1 to 24, wherein the antibody or antigen-binding fragment thereof that specifically binds to CD137 comprises a heavy chain variable region comprising the following CDRs:
a) GFNFGYSY [SEQ ID NO: 21] or an amino acid sequence containing up to 3 amino acid mutations compared to SEQ ID NO: 21, for example 1, 2 or 3 mutations;
b) IGSTSSHT [SEQ ID NO: 22] or an amino acid sequence containing up to 3 amino acid mutations compared to SEQ ID NO: 22, for example 1, 2 or 3 mutations; and
c) ARVYSSPGIDY [SEQ ID NO: 23] or an amino acid sequence containing up to 3 amino acid mutations compared to SEQ ID NO: 23, for example 1, 2 or 3 mutations.

34. A combination therapy according to claim 33, wherein the antibody or antigen-binding fragment thereof that specifically binds to CD137 comprises a heavy chain variable region comprising the CDRs of SEQ ID NOs 21, 22 and 23.

35. A combination therapy according to claim 34, wherein the antibody or antigen-binding fragment thereof that specifically binds to CD137 comprises a heavy chain variable region having the amino acid sequence of SEQ ID NO 19: or an amino acid sequence having at least 60% sequence identity therewith, for example at least 70%, 80%, 85%, 90%, 95%, 96%, 97%, 98% or 99% sequence identity.

36. A combination therapy according to any claims 1 to 24 or 33 to 35, wherein the antibody or antigen-binding fragment thereof that specifically binds to CD137 comprises a light chain variable region comprising the following CDRs:
a) QSIGST [SEQ ID NO: 24] or an amino acid sequence containing up to 3 amino acid mutations compared to SEQ ID NO: 24, for example 1, 2 or 3 mutations;
b) GAS [SEQ ID NO: 25] or an amino acid sequence containing up to 2 amino acid mutations compared to SEQ ID NO: 25; for example 1 or 2 mutations; and
c) QQYYTWVPFT [SEQ ID NO: 26] or an amino acid sequence containing up to 3 amino acid mutations compared to SEQ ID NO: 26, for example 1, 2 or 3 mutations.

37. A combination therapy according to claim 36, wherein the antibody or antigen-binding fragment thereof that specifically binds to CD137 comprises a light chain variable region comprising the CDRs of SEQ ID NOs: 24, 25 and 26.

38. A combination therapy according to claim 37, wherein the antibody or antigen-binding fragment thereof that specifically binds to CD137 comprises a light chain variable region having the amino acid sequence of SEQ ID NO: 20 or an amino acid sequence having at least 60% sequence identity therewith, for example at least 70%, 80%, 85%, 90%, 95%, 96%, 97%, 98% or 99% sequence identity.

39. A combination therapy according to any of claims 33 to 38, wherein the antibody or antigen-binding fragment thereof that specifically binds to CD137 comprises the CDRs of SEQ ID NOs: 21, 22, 23, 24, 25 and 26.

40. A combination therapy according to any of claims 33 to 39, wherein the antibody or antigen-binding fragment thereof that specifically binds to CD137 comprises a heavy chain variable region which comprises or consists of the amino acid sequence of SEQ ID NO: 19 and a light chain variable region which comprises or consists of the amino acid sequence of SEQ ID NO: 20.

41. A combination therapy according to any of the preceding claims, wherein the antibody or antigen-binding fragment thereof that specifically binds to CD137 comprises a heavy chain constant region, or part thereof.

42. A combination therapy according to claim 41 wherein the heavy chain constant region of the antibody or antigen-binding fragment thereof that specifically binds to CD137 is of an immunoglobulin subtype selected from the group consisting of IgG1, IgG2, IgG3 and IgG4.

43. A combination therapy according to claim 42 wherein the heavy chain constant region of the antibody or antigen-binding fragment thereof that specifically binds to CD137 is of an immunoglobulin subtype IgG4.

44. A combination therapy according to claim 43 wherein the heavy chain constant region of the antibody or antigen-binding fragment thereof that specifically binds to CD137 comprises or consists of an amino acid sequence selected from the group consisting of SEQ ID NO: 12, 13, 14 and 15.

45. A combination therapy according to any of the preceding claims, wherein the antibody or antigen-binding fragment thereof that specifically binds to CD137 comprises a light chain constant region, or part thereof.

46. A combination therapy according to claim 45 wherein the light chain constant region of the antibody or antigen-binding fragment thereof that specifically binds to CD137 is of a kappa or lambda light chain.

47. A combination therapy according to claim 46 wherein the light chain constant region of the antibody or antigen-binding fragment thereof that specifically binds to CD137 is of a kappa light chain.

48. A combination therapy according to claim 47 wherein the light chain constant region of the antibody or antigen-binding fragment thereof that specifically binds to CD137 comprises or consists of an amino acid sequence of SEQ ID NO: 16.

49. A combination therapy according to any of the preceding claims, wherein the antibody or antigen-binding fragment thereof that specifically binds to CD137 comprises an Fc region.

50. A combination therapy according to claim 49 wherein the Fc region of the antibody or antigen-binding fragment thereof that specifically binds to CD137 is naturally occurring.

51. A combination therapy according to claim 49 wherein the Fc region of the antibody or antigen-binding fragment thereof that specifically binds to CD137 is non-naturally occurring.

52. A combination therapy according to claim 51 wherein the Fc region of the antibody or antigen-binding fragment thereof that specifically binds to CD137 comprises mutations to shorten the half-life of the antibody or antigen binding fragment.

53. A combination therapy according to any one of the preceding claims, wherein the antibody or antigen-binding fragment thereof that specifically binds to CD137 comprises:
(a) a heavy chain comprising a variable region of SEQ ID NO: 1 together with a constant region of SEQ ID NO: 13, or an amino acid sequence having at least 60% sequence identity therewith, for example at least 70%, 80%, 85%, 90%, 95%, 96%, 97%, 98% or 99% sequence identity to SEQ ID NO: 1 and/or 13; and
(b) a light chain comprising a variable region of SEQ ID NO: 2 together with a constant region of SEQ ID NO: 16, or an amino acid sequence having at least 60% sequence identity therewith, for example at least 70%, 80%, 85%, 90%, 95%, 96%, 97%, 98% or 99% sequence identity to SEQ ID NO: 2 and/or 16.

54. A combination therapy according to any one of the preceding claims, wherein the antibody or antigen-binding fragment thereof that specifically binds to CD137 comprises:
(a) heavy chain comprising a variable region of SEQ ID NO: 19 together with a constant region of SEQ ID NO: 13 or an amino acid sequence having at least 60% sequence identity therewith, for example at least 70%, 80%, 85%, 90%, 95%, 96%, 97%, 98% or 99% sequence identity to SEQ ID NO: 19 or 13; and
(b) a light chain comprising a variable region of SEQ ID NO: 20 together with a constant region of SEQ ID NO: 16 or an amino acid sequence having at least 60% sequence identity therewith, for example at least 70%, 80%, 85%, 90%, 95%, 96%, 97%, 98% or 99% sequence identity to SEQ ID NO: 20 or 16.

55. A combination therapy according to any one of the preceding claims, wherein the antibody that specifically binds to CD137 is an intact IgG4 molecule comprising or consisting of two heavy chains having an amino acid sequence of SEQ ID NO: 17 and two light chains having an amino acid sequence of SEQ ID NO: 18, or an amino acid sequence having at least 60% sequence identity therewith, for example at least 70%, 80%, 85%, 90%, 95%, 96%, 97%, 98% or 99% sequence identity to SEQ ID NO: 17 and/or 18.

56. A combination therapy according to any one of the preceding claims, wherein the antibody that specifically binds to CD137 is an intact IgG4 molecule comprising or consisting of two heavy chains having an amino acid sequence of SEQ ID NO: 29 and two light chains having an amino acid sequence of SEQ ID NO: 30 or an amino acid sequence having at least 60% sequence identity therewith, for example at least 70%, 80%, 85%, 90%, 95%, 96%, 97%, 98% or 99% sequence identity to SEQ ID NO: 29 and/or 30.

57. A combination therapy according to any of the preceding claims, wherein the antibody or antigen-binding fragment thereof that specifically binds to CD137 further comprises a cytotoxic moiety.

58. A combination therapy according to claim 57 wherein the cytotoxic moiety comprises or consists of a radioisotope.

59. A combination therapy according to claim 57 wherein the cytotoxic moiety comprises or consists of a cytotoxic drug.

60. A combination therapy according to any of the preceding claims, wherein the antibody or antigen-binding fragment thereof that specifically binds to CD137 further comprises a detectable moiety.

61. A combination therapy according to claim 60 wherein the detectable moiety comprises or consists of a radioisotope.

62. A combination therapy according to any of claims 57 to 61 wherein the cytotoxic moiety and/or detectable moiety is joined to the antibody or antigen-binding fragment thereof that specifically binds to CD137 indirectly, via a linking moiety.

63. A combination therapy according to claim 62 wherein the linking moiety is a chelator.

64. A combination therapy according to claim 63 wherein the chelator is selected from the group consisting of derivatives of 1,4,7,10-tetraazacyclododecane-1,4,7,10,tetraacetic acid (DOTA), deferoxamine (DFO), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA).

65. A combination therapy according to any of claims 1 to 56 wherein the antibody or antigen-binding fragment that specifically binds to CD137 does not comprise a cytotoxic moiety or a detectable moiety.

66. A combination therapy according to any one of the preceding claims wherein said PD-1 inhibitor comprises or consists of an anti-PD-1 antibody, or antigen-binding fragment thereof capable of inhibiting PD-1 function.

67. A combination therapy according to claim 66 wherein the anti-PD-1 antibody is selected from the group consisting of Pembrolizumab, Nivolumab, Pidilizumab, Cemiplimab, AMP-224, PDR-001, MEDI-0680, JTX-4014 (Pimivalimab), Spartalizumab, Camrelizumab, Sintilimab, Tislelizumab, Toripalimab, Dostarlimab and INCMGA00012 (Retifanlimab), preferably wherein the anti-PD-1 antibody is Pembrolizumab or Nivolumab.

68. A combination therapy according to any one of the preceding claims wherein said PD-1 inhibitor comprises or consists of an anti-PD-L1 antibody, or antigen-binding fragment thereof capable of inhibiting PD-1 function.

69. A combination therapy according to claim 68 wherein the anti-PD-L1 antibody is selected from the group consisting of Atezolizumab (Tecentriq™, MPDL3280A), Durvalumab (MEDI-4736), Avelumab, MDX-1105, KN035 (Envafolimab) and CK-301 (Cosibelimab), preferably wherein the anti-PD-L1 antibody is Atezolizumab (Tecentriq™, MPDL3280A).

70. A combination therapy according to any one of the preceding claims, wherein the PD-1 inhibitor blocks the PD-1/PD-L1 interaction, optionally wherein the PD-1 inhibitor binds to PD-1 or PD-L1 in a manner that inhibits the ability of PD-L1 to bind to PD-1.

71. A combination therapy wherein the PD-1 inhibitor reactivate PD-1 expressing T cells, preferably by blocking the inhibitory signalling mediated by the tyrosine phosphatase SHP-2 that dephosphorylates signalling molecules downstream of the T cell receptor signalling molecules.

72. An antibody, or antigen-binding portion thereof, that specifically binds to CD137 for use in a method of treating a solid tumour,
wherein the antibody or antigen-binding portion thereof that specifically binds to CD137 is for use in combination with a further immunotherapeutic agent, wherein the further immunotherapeutic agent is a PD-1 inhibitor.

73. An antibody, or antigen-binding portion thereof, according to claim 72 wherein the antibody or antigen-binding portion thereof is as defined in any one of claims 5 to 65.

74. An antibody, or antigen-binding portion thereof, according to claim 72 or 73 wherein the PD-1 inhibitor is as defined in any one of claims 66 to 71.

75. Use of an antibody, or antigen-binding portion thereof, that specifically binds to CD137 in the preparation of a medicament for treating a solid tumour, wherein the antibody or antigen-binding portion thereof that specifically binds to CD137 is for use in combination with a further immunotherapeutic agent, wherein the further immunotherapeutic agent is a PD-1 inhibitor.

76. The use according to claim 75 wherein the antibody or antigen-binding portion thereof is as defined in any one of claims 5 to 65.

77. The use according to claim 75 or 76 wherein PD-1 inhibitor is as defined in any one of claims 66 to 71.

78. A pharmaceutical composition comprising (a) an antibody, or antigen-binding portion thereof, that specifically binds to CD137, and (b) a further immunotherapeutic agent, wherein the further therapeutic agent is a PD-1 inhibitor.

79. A pharmaceutical composition according to claim 78 wherein the antibody or antigen-binding portion thereof is as defined in any one of claims 5 to 65.

80. A pharmaceutical composition according to claim 78 or 79 wherein the PD-1 inhibitor is as defined in any one of claims 66 to 71.

81. A pharmaceutical composition according to any of claims 78 to 80 for use in medicine.

82. A pharmaceutical composition according to any of claims 78 to 81 for use in the treatment of cancer.

83. A kit for treating a solid tumour comprising (a) an antibody, or antigen-binding portion thereof, that specifically binds to CD137, and (b) a further immunotherapeutic agent, wherein the further immunotherapeutic agent is a PD-1 inhibitor.

84. A kit according to claim 83 wherein the antibody or antigen-binding portion thereof is as defined in any one of claims 5 to 65.

85. A kit according to claim 83 or 84 wherein the PD-1 inhibitor is as defined in any one of claims 66 to 71.

86. A method for treating a solid tumour in a subject, the method comprising administering to the subject a therapeutically effect amount of (a) administering to the subject a therapeutically effect amount of an antibody, or antigen-binding portion thereof, that specifically binds to CD137, and (b) administering to the subject a therapeutically effect amount of a further immunotherapeutic agent, wherein the further immunotherapeutic agent is a PD-1 inhibitor.

87. A method according to claim 86 wherein the antibody or antigen-binding portion thereof that specifically binds to CD137 is as defined in any one of claims 5 to 65.

88. A method according to claim 86 or 87 wherein the PD-1 inhibitor is as defined in any one of claims 66 to 71.

89. A method according to any one of claims 86 to 88 wherein steps (a) and (b) are carried out simultaneously or within 24 hours of each other.

90. A method according to any one of claims 86 to 89 wherein step (a) comprises systemic administration of the antibody to the tumour site.

91. A method according to any one of claims 86 to 90, wherein at least 30% of the amount of antibody administered in step (a) is retained at the tumour site at four hours after administration, preferably wherein at least 40% of the said amount is retained at the tumour site at four hours after administration.

92. A method according to any one of claims 86 to 91 wherein the further immunotherapeutic agent of step (b) is formulated as a composition suitable for systemic administration with at least one pharmaceutically acceptable diluent or carrier.

93. A method according to one of claims 86 to 92 wherein step (a) is conducted on multiple separate occasions and step (b) is conducted such that exposure of the subject to the further immunotherapeutic agent is continuous for the duration of the method.

94. A method according to any one of claims 86 to 93 wherein the subject is a human.

95. A kit comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof.

96. A kit according to claim 95 wherein the first and/or second isolated nucleic acid molecules are cDNA molecules.

97. A kit according to claim 95 or 96 wherein the first and/or second isolated nucleic acid molecules encode an antibody heavy chain or variable region thereof and/or encode an antibody light chain or variable region thereof.

98. A kit according to any of claims 95 to 97 wherein the first isolated nucleic acid molecule comprises or consists of a nucleotide sequence of SEQ ID NO: 9 and/or SEQ ID NO: 10.

99. A kit according to any of claims 95 to 97 wherein the first isolated nucleic acid molecule comprises or consists of a nucleotide sequence of SEQ ID NO: 27 and/or SEQ ID NO: 28.

100. A kit comprising a vector (such as an expression vector) comprising a first isolated nucleic acid molecule encoding an antibody or antigen-binding fragment thereof that specifically binds to CD137 or a component polypeptide chain thereof, and
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof,
optionally wherein the first isolated nucleic acid is as defined in any of claims 95 to 99.

101. A kit comprising a host cell (such as a mammalian cell, e.g. human cell, or Chinese hamster ovary cell, e.g. CHOK1SV cells) comprising a first isolated nucleic acid molecule encoding an antibody or antigen-binding fragment thereof that specifically binds to CD137 or a component polypeptide chain thereof, and:
(i) a PD-1 inhibitor; and/or
(ii) a second isolated nucleic acid molecule encoding an antibody of antigen-binding fragment thereof that specifically binds to PD-1 or PD-L1 or a component polypeptide chain thereof,
optionally wherein the first isolated nucleic acid is as defined in any of claims 95 to 99.

102. A combination therapy, method, use or kit as defined herein with reference to the description.
